(19) United States
(12) Patent Application Publication
(10) Pub. No.: US 2024/0150372 A1
(43) Pub. Date: May 9, 2024

(54) OXA-IBOGAINE ANALOGUES FOR TREATMENT OF SUBSTANCE USE DISORDERS

(71) Applicants: The Trustees of Columbia University in the City of New York, New York, NY (US); High Point University, High Point, NC (US)

(72) Inventors: Dalibor Sames, New York, NY (US); Vaclav Havel, Opava 9 (CZ); Scott Hemby, High Point, NC (US)

(73) Assignees: The Trustees of Columbia University in the City of New York, New York, NY (US); High Point University, High Point, NC (US)

(21) Appl. No.: 18/546,772

(22) PCT Filed: Feb. 17, 2022
(86) PCT No.: PCT/US2022/016757

§ 371 Date: Aug. 16, 2023

Related U.S. Application Data
(60) Provisional application No. 63/212,213, filed on Jun. 18, 2021.
(60) Provisional application No. 63/150,111, filed on Feb. 17, 2021.

(51) Int. Cl. C07D 491/22; A61P 25/36; A61P 29/00; C07D 471/22

(52) U.S. Cl. CPC C07D 491/22; A61P 25/36; A61P 29/00; C07D 471/22

(57) ABSTRACT

The present invention provides a method of treating a subject afflicted with a substance use disorder (SUD) comprising administering to the subject an effective amount of a compound having the structure:

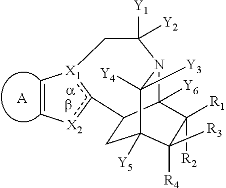

wherein
A is a ring structure, with or without substitution;
X₁ is C or N;
X₂ is N, O, or S;
Y₁ is H, -(alkyl), -(alkenyl), -(alkynyl), -(cycloalkyl), -(haloalkyl), -(alkyl)-O-(alkyl) or -(alkyl)-(cycloalkyl);
Y₂ is H, -(alkyl), -(alkenyl), -(alkynyl), -(cycloalkyl), -(haloalkyl), -(alkyl)-O-(alkyl) or -(alkyl)-(cycloalkyl);
Y₃ is H, -(alkyl), -(alkenyl), -(alkynyl), -(cycloalkyl), -(haloalkyl), -(alkyl)-O-(alkyl) or -(alkyl)-(cycloalkyl);
Y₄ is H, -(alkyl), -(alkenyl), -(alkynyl), -(cycloalkyl), -(haloalkyl), -(alkyl)-O-(alkyl) or -(alkyl)-(cycloalkyl);
Y₅ is H, -(alkyl), -(alkenyl), -(alkynyl), -(cycloalkyl), -(haloalkyl), -(alkyl)-O-(alkyl) or -(alkyl)-(cycloalkyl);
Y₆ is H, -(alkyl), -(alkenyl), -(alkynyl), -(cycloalkyl), -(haloalkyl), -(alkyl)-O-(alkyl) or -(alkyl)-(cycloalkyl);
α and β are each present or absent and when present each is a bond,
   wherein either α or β is present, and
   when α is present, then X₁ is C and X₂ is S or O, or
   when β is present, then X₁ is N and X₂ is N; and
R₁, R₂, R₃ and R₄ are each independently —H, -(alkyl), -(alkenyl), -(alkynyl), -(haloalkyl), -(cycloalkyl), -(aryl), -(heteroaryl), -(heteroalkyl), -(hydroxyalkyl), -(alkyl)-(aryl), -(alkyl)- (heteroaryl), -(alkyl)-(cycloalkyl), -(alkyl)-OH, -(alkyl)-O-(alkyl), —OH, —NH₂, —OAc, —CO₂H, —CN, OCF₃, halogen, —CO₂—(C₂-C₁₂ alkyl), C(O)—NH₂, —C(O)—NH-(alkyl), C(O)—NH-(aryl), —O-alkyl, —O-alkenyl, —O-alkynyl, —O-aryl, —O-(heteroaryl), —NH-alkyl, —NH-alkenyl, —NH-alkynyl, —NH-aryl, —NH-(heteroaryl), —O—C(O) (alkyl), or —C(O)—N(alkyl)₂,
   or a pharmaceutically acceptable salt or ester thereof, so as to thereby treat the subject afflicted with the substance use disorder (SUD).

This application claims priority of U.S. Provisional Application No. 63/212,213, filed Jun. 18, 2021, and U.S. Provisional Application No. 63/150,111, filed Feb. 17, 2021, the contents of which are hereby incorporated by reference.

This invention was made with government support under R01DA050613 awarded by the National Institutes of Health. The government has certain rights in the invention.

Throughout this application, certain publications are referenced in parentheses. Full citations for these publications may be found immediately preceding the claims. The disclosures of these publications in their entireties are hereby incorporated by reference into this application in order to describe more fully the state of the art to which this invention relates.

### BACKGROUND OF THE INVENTION

Ibogaine is the major psychoactive alkaloid found in the root bark of Tabernanthe iboga, a plant native to West Central Africa (Alper, K. R. 2001). The root bark has been used as a religious and healing sacrament by the native people in Africa owing to its distinct psychedelic post-acute effects. The clinical claims of ibogaine's anti-addictive properties, discovered in the U.S. in the 1960's, have largely been recapitulated in animal models of substance use disorders (SUDs), where ibogaine and its main metabolite, noribogaine, show several effects relevant to different aspects of SUDs (Glick, S. D. et al. 2001; Belgers, M. et al. 2016; Mash, D. C. et al. 2016).

However, the use of ibogaine has unfortunately been associated with sudden death in humans (Koenig, X. & Hilber, K. 2015), which has been attributed to adverse cardiac effects of ibogaine as well as its main active metabolite noribogaine (Glue et al 2016; Alper, K. et al 2016; Rubi, L. et al 2017), including QT interval prolongation and arrhythmias. QT prolongation is associated with an increased risk of life-threatening torsade de pointes (TdP) arrhythmias (Redfern, W. S. et al 2003). Both ibogaine and noribogaine are reported to block human ethera-go-go-related gene (hERG) potassium channels at clinically relevant low micromolar IC50 values (Alper, K. et al 2016), which can result in retardation of ventricular action potential (AP) repolarization and prolongation of the QT interval in the electrocardiogram (ECG) (Redfern, W. S. et al 2003). Additionally, it was shown that ibogaine and its active metabolite noribogaine significantly delayed action potential repolarization in human cardiomyocytes, which may result in a prolongation of the QT interval in the electrocardiogram and cardiac arrhythmias (Rubi, L. et al 2017). All of this suggests that ibogaine administration entails a significant risk of cardiac arrhythmia for humans.

Considering the large unmet needs in SUDs and psychiatric disorders in general, there is a strong impetus to study biological mechanisms that underpin ibogaine's effects, and to develop new analogs that increase ibogaine's safety and therapeutic index.

Novel classes of iboga analogs have been developed (U.S. Pat. No. 9,988,377; U.S. application Ser. Nos. 14/240,681, 15/528,339; PCT International Application No. PCT/US2012/052327, PCT/US2015/062726). The present invention shows that oxa-iboga analogs, defined as benzofuran-containing iboga analogs, exhibit profound, acute and long-lasting, and therapeutic-like effects in SUD rat models. Therefore, these analogs hold potential as novel therapeutics for SUDs, namely OUD and stimulant use disorder.

### SUMMARY OF THE INVENTION

The present invention provides a method of treating a subject afflicted with a substance use disorder (SUD) comprising administering to the subject an effective amount of a compound having the structure:

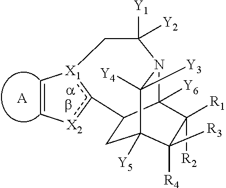

wherein
A is a ring structure, with or without substitution;
X₁ is C or N;
X₂ is N, O, or S;
Y₁ is H, -(alkyl), -(alkenyl), -(alkynyl), -(cycloalkyl), -(haloalkyl), -(alkyl)-O-(alkyl) or -(alkyl)-(cycloalkyl);
Y₂ is H, -(alkyl), -(alkenyl), -(alkynyl), -(cycloalkyl), -(haloalkyl), -(alkyl)-O-(alkyl) or -(alkyl)-(cycloalkyl);
Y₃ is H, -(alkyl), -(alkenyl), -(alkynyl), -(cycloalkyl), -(haloalkyl), -(alkyl)-O-(alkyl) or -(alkyl)-(cycloalkyl);
Y₄ is H, -(alkyl), -(alkenyl), -(alkynyl), -(cycloalkyl), -(haloalkyl), -(alkyl)-O-(alkyl) or -(alkyl)-(cycloalkyl);
Y₅ is H, -(alkyl), -(alkenyl), -(alkynyl), -(cycloalkyl), -(haloalkyl), -(alkyl)-O-(alkyl) or -(alkyl)-(cycloalkyl);
Y₆ is H, -(alkyl), -(alkenyl), -(alkynyl), -(cycloalkyl), -(haloalkyl), -(alkyl)-O-(alkyl) or -(alkyl)-(cycloalkyl);
α and β are each present or absent and when present each is a bond,
   wherein either α or β is present, and
   when α is present, then X, is C and X₂ is S or O, or
   when β is present, then X₁ is N and X₂ is N; and
R₁, R₂, R₃ and R₄ are each independently —H, -(alkyl), -(alkenyl), -(alkynyl), -(haloalkyl), -(cycloalkyl), -(aryl), -(heteroaryl), -(heteroalkyl), -(hydroxyalkyl), -(alkyl)-(aryl), -(alkyl)- (heteroaryl), -(alkyl)-(cycloalkyl), -(alkyl)-OH, -(alkyl)-O-(alkyl), —OH, —NH₂, —OAc, —CO₂H, —CN, OCF₃, halogen, —CO₂—(C₂-C₁₂ alkyl), C(O)—NH₂, —C(O)—NH-(alkyl), C(O)—NH-(aryl), —O-alkyl, —O-alkenyl, —O-alkynyl, —O-aryl, —O-(heteroaryl), —NH-alkyl, —NH-alkenyl, —NH-alkynyl, —NH-aryl, —NH-(heteroaryl), —O—C(O) (alkyl), or —C(O)—N(alkyl)₂,
   or a pharmaceutically acceptable salt or ester thereof, so as to thereby treat the subject afflicted with the substance use disorder (SUD).

The present invention also provides a compound having the structure:

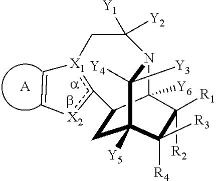

wherein
A is a ring structure, with or without substitution;
X₁ is C or N;
X₂ is N, O, or S;
Y₁ is H, -(alkyl), -(alkenyl), -(alkynyl), -(cycloalkyl), -(haloalkyl), -(alkyl)-O-(alkyl) or -(alkyl)-(cycloalkyl);
Y₂ is H, -(alkyl), -(alkenyl), -(alkynyl), -(cycloalkyl), -(haloalkyl), -(alkyl)-O-(alkyl) or -(alkyl)-(cycloalkyl);
Y₃ is H, -(alkyl), -(alkenyl), -(alkynyl), -(cycloalkyl), -(haloalkyl), -(alkyl)-O-(alkyl) or -(alkyl)-(cycloalkyl);
Y₄ is H, -(alkyl), -(alkenyl), -(alkynyl), -(cycloalkyl), -(haloalkyl), -(alkyl)-O-(alkyl) or -(alkyl)-(cycloalkyl);
Y₅ is H, -(alkyl), -(alkenyl), -(alkynyl), -(cycloalkyl), -(haloalkyl), -(alkyl)-O-(alkyl) or -(alkyl)-(cycloalkyl);
Y₆ is H, -(alkyl), -(alkenyl), -(alkynyl), -(cycloalkyl), -(haloalkyl), -(alkyl)-O-(alkyl) or -(alkyl)-(cycloalkyl);
α and β are each present or absent and when present each is a bond,
   wherein either α or β is present, and
   when α is present, then X₁ is C and X₂ is S or O, or
   when β is present, then X₁ is N and X₂ is N; and
R₁, R₂, R₃ and R₄ are each independently —H, -(alkyl), -(alkenyl), -(alkynyl), -(haloalkyl), -(cycloalkyl), -(aryl), -(heteroaryl), -(heteroalkyl), -(hydroxyalkyl), -(alkyl)-(aryl), -(alkyl)- (heteroaryl), -(alkyl)-(cycloalkyl), -(alkyl)-OH, -(alkyl)-O-(alkyl), —OH, —NH₂, —OAc, —CO₂H, —CN, OCF₃, halogen, —CO₂—(C₂-C₁₂ alkyl), C(O)—NH₂, —C(O)—NH-(alkyl), C(O)—NH-(aryl), —O-alkyl, —O-alkenyl, —O-alkynyl, —O-aryl, —O-(heteroaryl), —NH-alkyl, —NH-alkenyl, —NH-alkynyl, —NH-aryl, —NH-(heteroaryl), —O—C(O) (alkyl), or —C(O)—N(alkyl)₂,
   or a pharmaceutically acceptable salt or ester thereof.

### BRIEF DESCRIPTION OF THE FIGURES

FIG. 1: Pro-arrhythmia assay analysis of A/ noribogaine (n=6) B/oxa-noribogaine (n=5) C/ epi-oxa-noribogaine (n=6) and D/ desethyl-oxa-noribogaine (n=5).

FIG. 2: Comparison of the effects of oxa-noribogaine and noribogaine on morphine self-administration in rats. Morphine (10 μg/infusion) engendered and maintained intravenous self-administration in male F344 rats during sessions of 2 hr of maximum of 50 infusions. Rats were randomly assigned to received either oxa-noribogaine (40 mg/kg, IP; n=8) or noribogaine (40 mg/kg, IP; n=10). The number of infusions obtained following VEH administration did not significantly differ from baseline for either group. A two-way repeated measures ANOVA for the first five sessions following administration revealed a main effect of Sessions [F (2.890, 40.46)=28.80; p<0.0001], Drug [F (1, 14)=33.07, p<0.0001] and a Session x Drug Interaction [F (4, 56)=6.317, p=0.0003]. Tukey's post hoc analysis revealed no significant difference on Session 1, but significant differences in the number of infusions obtained for sessions 2-5 (p<0.05). t-tests were used to compare the number of infusions at baseline with the time points following administration of each drug. * indicates p<0.05

FIG. 3: Dose comparison of oxa-noribogaine effects on morphine self-administration. Morphine (10 μg/infusion) engendered and maintained intravenous self-administration in male F344 rats during sessions of 2 hr of maximum of 50 infusions. Rats were randomly assigned to received either 10 or 40 mg/kg of oxa-noribogaine (IP; n=7, n=8, respectively). The number of infusions obtained following VEH administration did not significantly differ from baseline for either group. A two-way repeated measures ANOVA for the first four sessions following administration revealed a main effect of Sessions [F (2.385, 30.21)=28.42, P<0.001], Dose [F (1, 13)=78.57, p<0.0001] and a Session x Dose Interaction [F (3, 38)=12.87, p<0.0001]. Tukey's post hoc analysis revealed no significant difference between groups on Session 1, but significant differences in the number of infusions obtained for sessions 2-4 (p<0.01). t-tests were used to compare the number of infusions at baseline with the time points following administration of each drug. * indicates p<0.05.

FIG. 4: Comparison of the effects of oxa-noribogaine and epi-oxa-noribogaine on morphine self-administration. Morphine (10 μg/infusion) engendered and maintained intravenous self-administration in male F344 rats during sessions of 2 hr of maximum of 50 infusions. Rats were randomly assigned to received either oxa-noribogaine (40 mg/kg, IP; n=8) or epi-oxa-noribogaine (40 mg/kg, IP; n=10). The number of infusions obtained following VEH administration did not significantly differ from baseline for either group. A two-way repeated measures ANOVA for the first eight sessions following administration revealed a main effect of Sessions [F (4.016, 63.68)=20.62, p<0.0001], Drug [F (1, 16)=27.64, p<0.0001] and a Session x Drug Interaction [F (7, 111)=3.908, p=0.0008]. Tukey's post hoc analysis revealed no significant difference on Session 1, but significant differences in the number of infusions obtained for sessions 2-5 (p<0.05). t-tests were used to compare the number of infusions at baseline with the time points following administration of each drug. * indicates p<0.05.

FIG. 5: Effect of oxa-noribogaine on cocaine and fentanyl self-administration. A/ Cocaine (0.625 μg/infusion; n=4) engendered and maintained intravenous self-administration in male F344 rats during sessions of 2 hr of maximum of 50 infusions. The number of infusions obtained following VEH administration did not significantly differ from baseline. Two-tailed t-test revealed significant differences between the number of infusions obtained following VEH versus the first three sessions following administration of oxa-noribogaine (40 mg/kg, IP; *p<0.05). B/ Fentanyl (0.625 μg/infusion; n=3) engendered and maintained intravenous self-administration in male F344 rats during sessions of 2 hr of maximum of 50 infusions. The number of infusions obtained following VEH administration did not significantly differ from baseline. Two-tailed t-test revealed significant differences between the number of infusions obtained following VEH versus the first and second sessions following administration of oxa-noribogaine (40 mg/kg, IP; *p<0.05).

FIG. 6: Effect of repeated dosing regimen of oxa-noribogaine on morphine self-administration. Morphine (10 μg/infusion) engendered and maintained intravenous self-administration in male F344 rats during daily sessions of 2 hr of maximum of 50 infusions. Following the establishment of stable responding, rats received oxa-noribogaine (40, 10 and 5 mg/kg, IP; n=11, respectively). The number of infusions obtained following VEH administration did not significantly differ from baseline for either group. Five days following VEH administration, a repeated administration procedure of oxa-noribogaine was undertaken as follows: Days 1 and 6: 40 mg/kg, Days 11, 15 and 17: 10 mg/kg, and Days 19, 21, and 23: 5 mg/kg. The respective doses of oxa-noribogaine were administered 15 min prior to the experimental sessions indicated above. t-tests were used to compare the number of infusions at baseline with the time points following administration of each drug. The number of infusions obtained in experimental sessions were significantly lower than VEH at all time points following the initial oxa-noribogaine administration.

FIG. 7: Oxa-noribogaine affects severe fentanyl addiction states in rats. A/ A schematic experimental design for the intermittent access fentanyl SA paradigm, including progressive ratio probe sessions (PR), intermittent fentanyl access sessions (IntA-SA), and a dosing regimen including reset (40 mg/kg) and maintenance (10 mg/kg) doses of oxa-noribogaine. Dosing begins after extended period of fentanyl IntA-SA known to induce severe addiction states. B/ Repeated dosing of oxa-noribogaine suppresses fentanyl self-administration across sessions compared to vehicle (P<0.0001). On the days of administration, we observed a significant reduction or a trend towards significant reduction in fentanyl self-administration. C/ Fentanyl SA induced significant mechanical hyperalgia in fentanyl-dependent subjects (P<0.0001) as measured by the electronic von Frey test, an effect alleviated by repeated oxa-noribogaine administration (Session 50 compared to Pre-Inj (21); P=0.0047). An acute oxa-noribogaine challenge (Session 53; 10 mg/kg) reversed the fentanyl-induced mechanical allodynia in the oxa-noribogaine group (P=0.0022). Fentanyl subjects administered oxa-noribogaine were subdivided into D/ high responders (>100 infusions/session; N=4) and E/ low responders (<100 infusions/session; N=7). Compared to the vehicle group, oxa-noribogaine suppressed fentanyl intake across sessions for both the high and low responders (P<0.0001). Analysis of fentanyl intake on the day of injection and subsequent post-injection sessions (4 sessions following 40 mg/kg and 1 day following 10 mg/kg) was conducted. Intake was reduced following administration of 40 mg/kg in both groups, and remained suppressed following all 10 mg/kg injections in the low responder group. Error bars represent the mean±SEM, *P<0.05, **P<0.01, ***P<0.001, ****P<0.0001.

FIG. 8: Fentanyl intermittent access self-administration in rats. A/ Detailed experimental design involving continuous access SA, intermittent fentanyl access SA (IntA-SA), progressive ratios (PR), von Frey (VF), and body mass measurements at indicated points. B/Repeated administration of oxa-noribogaine has no effect on the body weight of test subjects. C/ Operant food intake is acutely inhibited by oxa-noribogaine but food responding returns next day. D/ Short PR probes showed a trend toward an increased breaking point values after the intermittent access module and decreased breaking point values after the oxa-noribogaine treatment. E/ Von Frey measurements indicate an acute and long-lasting effect of oxa-noribogaine alleviating mechanical allodynia induced by daily fentanyl intake. F/ Selected examples of individual subjects treated with oxa-noribogaine in comparison to vehicle treatment group. Subject #739 is a high fentanyl intake subject that shows a strong and lasting response to oxa-noribogaine treatment in terms of both fentanyl intake reduction and a dramatic drop in PR breaking point (pre-treatment PR2 and PR3 16 days after the last dose), a measure of reinforcing efficacy of fentanyl (or motivation for fentanyl); subject #753 shows a strong and lasting response; subject #746 a moderate but increasing efficacy with repeated dosing and a marked drop in PR breaking point; subject #756 is a high intake subject showing relatively poor response in intake but a dramatic drop in PR breaking point; subject #760 shows a good acute response but poor long term effects and PR breaking point increase; and #741 is a very high fentanyl intake subject with a strong acute response to oxa-noribogaine but a poor long-term effect and a moderate PR breaking point increase. 3 out of 4 subjects with high values of PR breaking point prior treatment (>100, #739, 746, and 756) showed a marked decrease in breaking point value post treatment (228%, 659%, and 47% respectively); the 4^{th} subject showed a modest increase (#741, 12%). 2 out of 2 subjects in the vehicle cohort with breaking point values >100 showed no change or increase in the breaking point values. Error bars represent the mean±SEM, *P<0.05, **P<0.01, ***P<0.001, ****P<0.0001.

FIG. 9: Within session fentanyl intake following oxa-noribogaine administration. Panels represent fentanyl infusions following administration of oxa-noribogaine and vehicle (mean±SEM) for 12 5-min bins per daily 6 h self-administration session after A/ Sessions 21-25: 40 mg/kg oxa-noribogaine or vehicle, B/ Sessions 26-27: 10 mg/kg oxa-noribogaine or vehicle, C/ Sessions 28-29: 10 mg/kg oxa-noribogaine or vehicle, D/ Sessions 30-31: 10 mg/kg oxa-noribogaine or vehicle. E/ Sessions 32-33: 10 mg/kg oxa-noribogaine or vehicle, F/ Sessions 34-35: 10 mg/kg oxa-noribogaine or vehicle, G/ Sessions 36-37: 10 mg/kg oxa-noribogaine or vehicle and H/ Sessions 52-53: challenge of 10 mg/kg oxa-noribogaine or vehicle. A significant difference was observed between the oxa-noribogaine and vehicle groups in the number of infusions across the session on Sessions 21 (P<0.01). The number of infusions was significantly reduced following oxa-noribogaine for bins 3-12. No difference was observed on Sessions 22-25. While no significant difference was observed between the groups following the second injection (Sessions 26 or 27), oxa-noribogaine (10 mg/kg) administration resulted in a significant decrease in the number of infusions on the day of injection (Sessions 28: P=0.0088; Sessions 30: P=0.0041; Sessions 32: P=0.93; Sessions 34: P<0.001; Sessions 36, P=0.0119; Sessions 52: P=0.002). Intake is not statistically different from vehicle on the day(s) following oxa-noribogaine administration (day-after effect), but a clear trend is seen in bins 1 after repeated administration. I/ Latency to the first infusion in the first 5-min bin by Sessions. Data represent the mean±SEM, specific statistical tests, information on reproducibility, and P values are reported in Methods and in Supplementary Statistics Table, *P<0.05, **P<0.01, #P<0.001, **P<0.0001.

FIG. 10: Comparison of the effects of oxa-noribogaine on morphine (data shown on FIG. 6) and fentanyl (same experimental design as described on FIG. 5B) self-administration. A single injection of oxa-noribogaine (40 mg/kg) results in statistically significant suppression of fentanyl intake for 4 days, with suppression trends observable for at least 5 days.

FIG. 11: (16R)-oxa-noribogaine induce potent analgesia in the mouse tail-flick test (ED₅₀=2.8 mg/kg), in contrast to the less active (16S) enantiomer (ED₅₀>30 mg/kg).

FIG. 12: A/ Increased levels of mature BDNF protein in the medial prefrontal cortex (mPFC) after a single dose of either oxa-noribogaine (40 mg/kg; IP) or noribogaine (40 mg/kg; IP) were detected after 24 h (OXA1 and NOR1) and remained elevated for up to 5 days (OXA5 and NOR5). Administration of oxa-noribogaine (40 mg/kg; IP) but not noribogaine (40 mg/kg; IP) significantly increased GDNF protein levels in B/ mPFC and C/ ventral tegmental area (VTA) after 5 days (OXA5). D/ No statistically significant modulation of neurothropic factor expression was detected in the nucleus accumbens (NAc).

FIG. 13: A/ Chiral SFC and B-C/ LC-MS analysis of racemic ibogamine.

FIG. 14: A/ Chiral SFC and B/ LC-MS analysis of (16R)-ibogamine.

FIG. 15: A/ Chiral SFC and B-C/ LC-MS analysis of (16S)-ibogamine.

FIG. 16: A/ Chiral SFC and B-C/ LC-MS analysis of racemic oxa-noribogaine.

FIG. 17: A/ Chiral SFC and B/ LC-MS analysis of (16S)-oxa-noribogaine.

FIG. 18: A/ Chiral SFC and B/ LC-MS analysis of (16R)-oxa-noribogaine.

FIG. 19: Structures of novel noribogaine analogs and known indole alkaloids used for the assignment of their configuration.

FIG. 20: X-ray structure of a single enantiomer of oxa-noribogaine, color coding of atoms: carbon—gray; hydrogen—white; oxygen—red; nitrogen—blue; chloride—green.

FIG. 21: A/ Absorption spectra for indole and benzofuran alkaloids, B/ CD spectra for selected indole alkaloids, C/ CD spectra for synthetic ibogamine enantiomers, and D/ CD spectra for noribogaine and its oxa-analogs.

### DETAILED DESCRIPTION OF THE INVENTION

The present invention provides a method of treating a subject afflicted with a substance use disorder (SUD) comprising administering to the subject an effective amount of a compound having the structure:

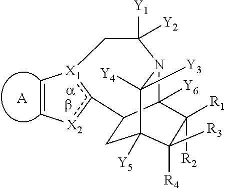

wherein
A is a ring structure, with or without substitution;
X₁ is C or N;
X₂ is N, O, or S;
Y₁ is H, -(alkyl), -(alkenyl), -(alkynyl), -(cycloalkyl), -(haloalkyl), -(alkyl)-O-(alkyl) or -(alkyl)-(cycloalkyl);
Y₂ is H, -(alkyl), -(alkenyl), -(alkynyl), -(cycloalkyl), -(haloalkyl), -(alkyl)-O-(alkyl) or -(alkyl)-(cycloalkyl);
Y₃ is H, -(alkyl), -(alkenyl), -(alkynyl), -(cycloalkyl), -(haloalkyl), -(alkyl)-O-(alkyl) or -(alkyl)-(cycloalkyl);
Y₄ is H, -(alkyl), -(alkenyl), -(alkynyl), -(cycloalkyl), -(haloalkyl), -(alkyl)-O-(alkyl) or -(alkyl)-(cycloalkyl);
Y₅ is H, -(alkyl), -(alkenyl), -(alkynyl), -(cycloalkyl), -(haloalkyl), -(alkyl)-O-(alkyl) or -(alkyl)-(cycloalkyl);
Y₆ is H, -(alkyl), -(alkenyl), -(alkynyl), -(cycloalkyl), -(haloalkyl), -(alkyl)-O-(alkyl) or -(alkyl)-(cycloalkyl);
α and β are each present or absent and when present each is a bond,
   wherein either α or β is present, and
   when α is present, then X, is C and X₂ is S or O, or
   when β is present, then X₁ is N and X₂ is N; and
R₁, R₂, R₃ and R₄ are each independently —H, -(alkyl), -(alkenyl), -(alkynyl), -(haloalkyl), -(cycloalkyl), -(aryl), -(heteroaryl), -(heteroalkyl), -(hydroxyalkyl), -(alkyl)-(aryl), -(alkyl)- (heteroaryl), -(alkyl)-(cycloalkyl), -(alkyl)-OH, -(alkyl)-O-(alkyl), —OH, —NH₂, —OAc, —CO₂H, —CN, OCF₃, halogen, —CO₂—(C₂-C₁₂ alkyl), C(O)—NH₂, —C(O)—NH-(alkyl), C(O)—NH-(aryl), —O-alkyl, —O-alkenyl, —O-alkynyl, —O-aryl, —O-(heteroaryl), —NH-alkyl, —NH-alkenyl, —NH-alkynyl, —NH-aryl, —NH-(heteroaryl), —O—C(O) (alkyl), or —C(O)—N(alkyl)₂,
   or a pharmaceutically acceptable salt or ester thereof, so as to thereby treat the subject afflicted with the substance use disorder (SUD).

The above structure refers to a racemic mixture.

In some embodiments of the above method, wherein the compound has the structure:

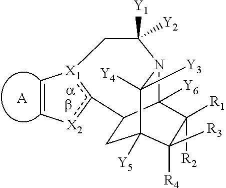

or a pharmaceutically acceptable salt or ester thereof.

The above structure refers to a racemic mixture.

In some embodiments of the above method, wherein the substance use disorder is opioid use disorder, alcohol use disorder or stimulant use disorder including nicotine use disorder.

In some embodiments of the above methods, wherein the substance is an opioid.

In some embodiments of any of the above methods, wherein the opioid is morphine, hydromorphone, oxymorphone, codeine, dihydrocodeine, hydrocodone, oxycodone, nalbuphine, butorphanol, etorphine, dihydroetorphine, levorphanol, metazocine, pentazocine, meptazinol, meperidine (pethidine), buprenorphine, methadone, tramadol, tapentadol, mitragynine, 3-deutero-mitragynine, 7-hydroxymitragynine, 3-deutero-7-hydroxymitragynine, mitragynine pseudoindoxyl or tianeptine.

In some embodiments of any of the above methods, wherein the opioid is fentanyl, sufentanil, alfentanil, furanylfentanyl, 3-methylfentanyl, valerylfentanyl, butyrylfentanyl, -Hydroxythiofentanyl, acrylfentanyl or carfentanil.

In some embodiments of any of the above methods, wherein the stimulant is cocaine, amphetamine, methamphetamine or cathinone and its derivatives.

In some embodiments of any of the above methods, wherein the stimulant is nicotine.

In some embodiments of any of the above methods, which comprises treating a symptom of substance use disorder.

In some embodiments of any of the above methods, wherein a symptom of substance use disorder is opioid withdrawal.

In some embodiments of any of the above methods, wherein a symptom of substance use disorder is hyperalgesia or allodynia.

In some embodiments of any of the above methods, wherein a symptom of substance use disorder is hyperalgesia. In some embodiments of any of the above methods, wherein a symptom of substance use disorder is allodynia.

In some embodiments of any of the above methods, wherein the risk of relapse to the use of opioids, alcohol or stimulants is reduced.

In some embodiments of any of the above methods, wherein self-administration of an opioid, alcohol or stimulant is reduced.

In an embodiment of any of the above methods, wherein self-administration of an opioid is reduced.

In an embodiment of any of the above methods, wherein self-administration of alcohol is reduced.

In an embodiment of any of the above methods, wherein self-administration of a stimulant is reduced.

In an embodiment of any of the above methods, wherein self-administration of morphine is reduced. In another embodiment of any of the above methods, wherein self-administration of fentanyl is reduced. In another embodiment of any of the above methods, wherein self-administration of cocaine is reduced.

In some embodiments of any of the above methods, wherein the treating is effective for an extended time.

In some embodiments of any of the above methods, wherein the time is 1-5 days.

In some embodiments of any of the above methods, wherein the time is 1-5 weeks.

In some embodiments of any of the above methods, wherein the effective amount of the compound administered to the subject without inducing cardiotoxicity.

In some embodiments of any of the above methods, wherein the effective amount of the compound administered to the subject without inducing QT interval prolongation.

In some embodiments of any of the above methods, wherein the effective amount of the compound administered to the subject without inducing cardiac arrhythmia.

In some embodiments of any of the above methods, wherein the subject is a mammal.

In some embodiments of any of the above methods, wherein the mammal is a human.

In some embodiments of any of the above methods, wherein the compound has the structure:

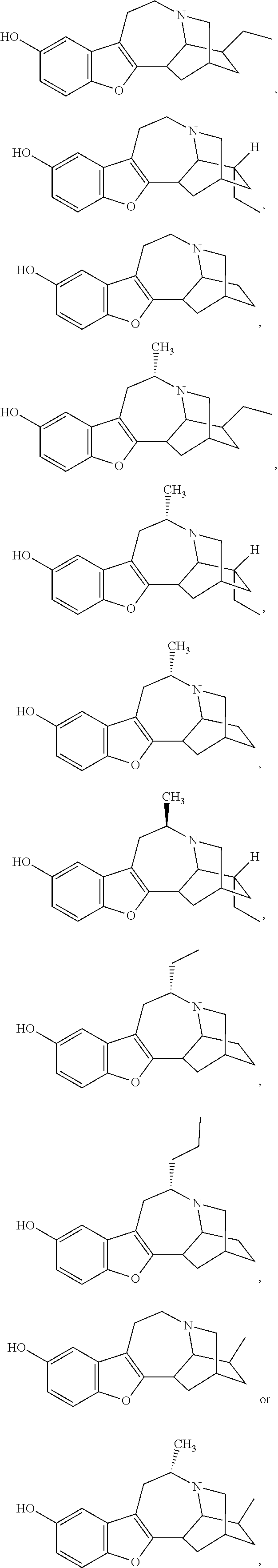

or a pharmaceutically acceptable salt or ester thereof.

The above structures refer to a racemic mixture.

In some embodiments of any of the above methods, wherein the compound has the structure:

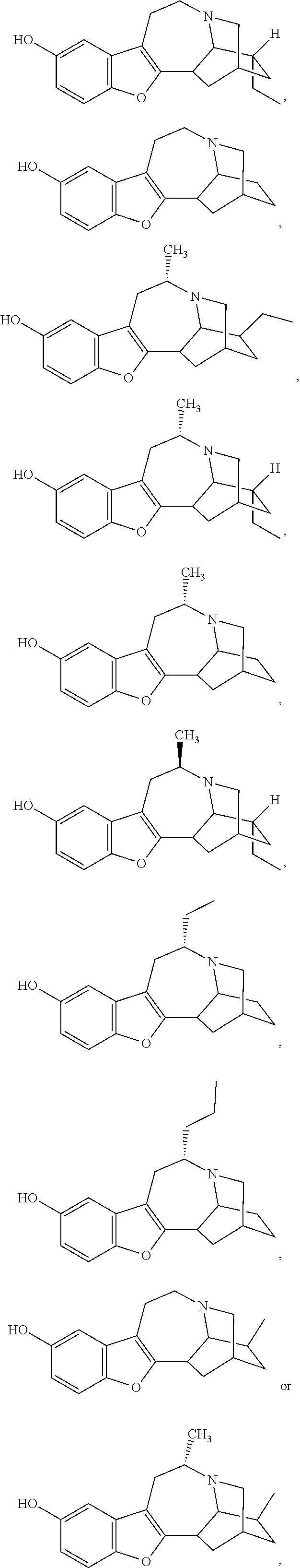

or a pharmaceutically acceptable salt or ester thereof.

The above structures refer to a racemic mixture.

In some embodiments of any of the above methods, wherein the effective amount of 10-500 mg of the compound is administered to the subject.

In some embodiments of any of the above methods, comprising administering a pharmaceutical composition, which comprises the compound and a pharmaceutically acceptable carrier.

The present invention provides a compound having the structure:

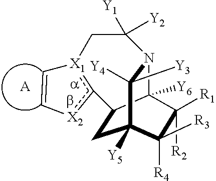

wherein
A is a ring structure, with or without substitution;
X₁ is C or N;
X₂ is N, O, or S;
Y₁ is H, -(alkyl), -(alkenyl), -(alkynyl), -(cycloalkyl), -(haloalkyl), -(alkyl)-O-(alkyl) or -(alkyl)-(cycloalkyl);
Y₂ is H, -(alkyl), -(alkenyl), -(alkynyl), -(cycloalkyl), -(haloalkyl), -(alkyl)-O-(alkyl) or -(alkyl)-(cycloalkyl);
Y₃ is H, -(alkyl), -(alkenyl), -(alkynyl), -(cycloalkyl), -(haloalkyl), -(alkyl)-O-(alkyl) or -(alkyl)-(cycloalkyl);
Y₄ is H, -(alkyl), -(alkenyl), -(alkynyl), -(cycloalkyl), -(haloalkyl), -(alkyl)-O-(alkyl) or -(alkyl)-(cycloalkyl);
Y₅ is H, -(alkyl), -(alkenyl), -(alkynyl), -(cycloalkyl), -(haloalkyl), -(alkyl)-O-(alkyl) or -(alkyl)-(cycloalkyl);
Y₆ is H, -(alkyl), -(alkenyl), -(alkynyl), -(cycloalkyl), -(haloalkyl), -(alkyl)-O-(alkyl) or -(alkyl)-(cycloalkyl);
α and β are each present or absent and when present each is a bond,
   wherein either α or β is present, and
   when α is present, then X₁ is C and X₂ is S or O, or
   when β is present, then X₁ is N and X₂ is N; and
R₁, R₂, R₃ and R₄ are each independently —H, -(alkyl), -(alkenyl), -(alkynyl), -(haloalkyl), -(cycloalkyl), -(aryl), -(heteroaryl), -(heteroalkyl), -(hydroxyalkyl), -(alkyl)-(aryl), -(alkyl)- (heteroaryl), -(alkyl)-(cycloalkyl), -(alkyl)-OH, -(alkyl)-O-(alkyl), —OH, —NH₂, —OAc, —CO₂H, —CN, OCF₃, halogen, —CO₂—(C₂-C₁₂ alkyl), C(O)—NH₂, —C(O)—NH-(alkyl), C(O)—NH-(aryl), —O-alkyl, —O-alkenyl, —O-alkynyl, —O-aryl, —O-(heteroaryl), —NH-alkyl, —NH-alkenyl, —NH-alkynyl, —NH-aryl, —NH-(heteroaryl), —O—C(O) (alkyl), or —C(O)—N(alkyl)₂,
   or a pharmaceutically acceptable salt or ester thereof.

The above structure refers to a specific enantiomer.

In some embodiments, the compound having the structure:

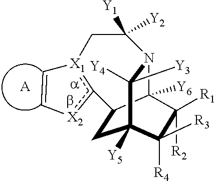

or a pharmaceutically acceptable salt or ester thereof.

The above structure refers to a specific enantiomer.

In some embodiments of any of the above methods or in some embodiments, the compound having the structure:

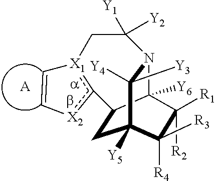

wherein
A is a ring structure, with or without substitution;
X₁ is C or N;
X₂ is N, O or S;
Y₁ is H, -(alkyl), -(alkenyl), -(alkynyl), -(cycloalkyl) or -(alkyl)-(cycloalkyl);
Y₂ is H, -(alkyl), -(alkenyl), -(alkynyl), -(cycloalkyl) or -(alkyl)-(cycloalkyl);
Y₃ is H, -(alkyl), -(alkenyl), -(alkynyl), -(cycloalkyl) or -(alkyl)-(cycloalkyl);
Y₄ is H, -(alkyl), -(alkenyl), -(alkynyl), -(cycloalkyl) or -(alkyl)-(cycloalkyl);
Y₅ is H, -(alkyl), -(alkenyl), -(alkynyl), -(cycloalkyl) or -(alkyl)-(cycloalkyl);
Y₆ is H, -(alkyl), -(alkenyl), -(alkynyl), -(cycloalkyl) or -(alkyl)-(cycloalkyl);
α and β are each present or absent and when present each is a bond,
   wherein either α or β is present, and
   when α is present, then X₁ is C and X₂ is S or O, or
   when β is present, then X₁ is N and X₂ is N; and
R₁, R₂, R₃ and R₄ are each independently H, -(alkyl), -(alkenyl), -(alkynyl), -(aryl), -(heteroaryl), -(heteroalkyl), -(hydroxyalkyl), -(alkyl)-(aryl), -(alkyl)-(heteroaryl), -(alkyl)-OH, -(alkyl)- O-(alkyl), —OH, —NH₂, —CO₂H, —CO₂—(C₂-C₁₂ alkyl) or —C(O)—NH-(alkyl),
   or a pharmaceutically acceptable salt or ester thereof.

The above structure refers to a specific enantiomer.

In some embodiments of any of the above methods or in some embodiments, the compound having the structure:

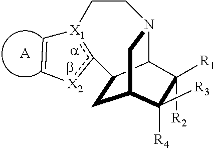

wherein
A is a ring structure, with or without substitution;
X₁ is C or N;
X₂ is N, O or S;
α and β are each present or absent and when present each is a bond,
   wherein either α or β is present, and
   when α is present, then X₁ is C and X₂ is S or O, or
   when β is present, then X₁ is N and X₂ is N; and
R₁, R₂, R₃ and R₄ are each independently H, -(alkyl), -(alkenyl), -(alkynyl), -(aryl), -(heteroaryl), -(heteroalkyl), -(hydroxyalkyl), -(alkyl)-(aryl), -(alkyl)-(heteroaryl), -(alkyl)-OH, -(alkyl)- O-(alkyl), —OH, —NH₂, —CO₂H, —CO₂—(C₂-C₁₂ alkyl) or —C(O)—NH-(alkyl),
   or a pharmaceutically acceptable salt or ester thereof.

The above structure refers to a specific enantiomer.

In some embodiments of any of the above methods or in some embodiments, the compound having the structure:

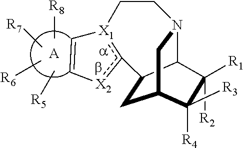

wherein
A is an aryl or heteroaryl;
X₁ is C or N;
X₂ is N, O or S;
α and β are each present or absent and when present each is a bond,
   wherein either α or β is present,
   when α is present, then X₁ is C and X₂ is S or O, and
   when β is present, then X₁ is N and X₂ is N;
R₁, R₂, R₃ and R₄ are each independently H, -(alkyl), -(alkenyl), -(alkynyl), -(aryl), -(heteroaryl), -(heteroalkyl), -(hydroxyalkyl), -(alkyl)-(aryl), -(alkyl)-(heteroaryl), -(alkyl)-OH, -(alkyl)- O-(alkyl), —OH, —NH₂, —CO₂H, —CO₂—(C₂-C₁₂ alkyl), or —C(O)—NH-(alkyl); and
R₅, R₆, R₇, R₈ are each independently —H, halogen, —CN, —CF₃, —OCF₃, -(alkyl), -(alkenyl), -(alkynyl), -(aryl), -(heteroaryl), -(heteroalkyl), -(hydroxyalkyl), —NH₂, —NH-(alkyl), —NH-(alkenyl), —NH-(alkynyl) —NH-(aryl), —NH-(heteroaryl), —OH, —OAc, —CO₂H, —CO₂-(alkyl), —O—C(O) (alkyl), —O-(alkyl), —O-(alkenyl), —O-(alkynyl), —O-(aryl), —O-(heteroaryl), —C(O)—NH₂, —C(O)—NH-(alkyl) or —C(O)—NH-(aryl),
   or a pharmaceutically acceptable salt or ester thereof.

The above structure refers to a specific enantiomer.

In some embodiments of any of the above methods or in some embodiments, the compound having the structure:

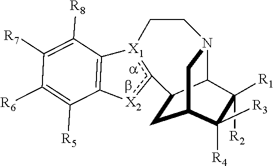

wherein
X₁ is C or N;
X₂ is N, O or S;
a and are each present or absent and when present each is a bond,
   wherein either α or β is present,
   when α is present, then X₁ is C and X₂ is S or O, and
   when β is present, then X₁ is N and X₂ is N;
R₁, R₂, R₃ and R₄ are each independently H, -(alkyl), -(alkenyl), -(alkynyl), -(aryl), -(heteroaryl), -(heteroalkyl), -(hydroxyalkyl), -(alkyl)-(aryl), -(alkyl)-(heteroaryl), -(alkyl)-OH, -(alkyl)- O-(alkyl), —OH, —NH₂, —CO₂H, —CO₂—(C₂-C₁₂ alkyl) or —C(O)—NH-(alkyl); and
R₅, R₆, R₇, R₈ are each independently —H, halogen, —CN, —CF₃, —OCF₃, -(alkyl), -(alkenyl), -(alkynyl), -(aryl), -(heteroaryl), -(heteroalkyl), -(hydroxyalkyl), —NH₂, —NH-(alkyl), —NH-(alkenyl), —NH-(alkynyl) —NH-(aryl), —NH-(heteroaryl), —OH, —OAc, —CO₂H, —CO₂-(alkyl), —O—C(O) (alkyl), —O-(alkyl), —O-(alkenyl), —O-(alkynyl), —O-(aryl), —O-(heteroaryl), —C(O)—NH₂, —C(O)—NH-(alkyl) or —C(O)—NH-(aryl),
   or a pharmaceutically acceptable salt or ester thereof.

The above structure refers to a specific enantiomer.

In some embodiments of any of the above methods or in some embodiments, the compound having the structure:

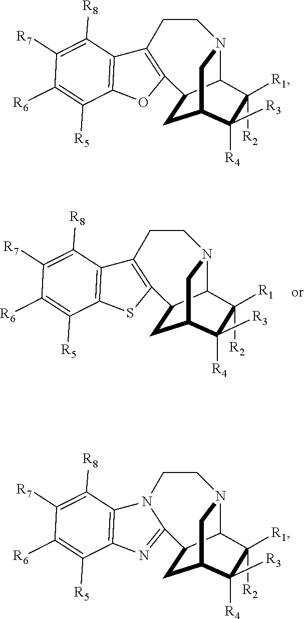

or a pharmaceutically acceptable salt or ester thereof.

The above structures refer to a specific enantiomer.

In some embodiments of any of the above methods or in some embodiments, the compound having the structure:

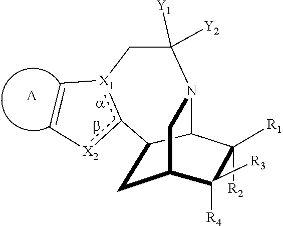

wherein
A is a ring structure, with or without substitution;
X₁ is C or N;
X₂ is N, O or S;
Y₁ is H, -(alkyl), -(alkenyl) or -(alkynyl);
Y₂ is H, -(alkyl), -(alkenyl) or -(alkynyl);
α and β are each present or absent and when present each is a bond,
   wherein either α or β is present, and
   when α is present, then X₁ is C and X₂ is S or O, or
   when β is present, then X₁ is N and X₂ is N; and
R₁, R₂, R₃ and R₄ are each independently H, -(alkyl), -(alkenyl), -(alkynyl), -(aryl), -(heteroaryl), -(heteroalkyl), -(hydroxyalkyl), -(alkyl)-(aryl), -(alkyl)-(heteroaryl), -(alkyl)-OH, -(alkyl)- O-(alkyl), —OH, —NH₂, —CO₂H, —CO₂—(C₂-C₁₂ alkyl) or —C(O)—NH-(alkyl),
   or a pharmaceutically acceptable salt or ester thereof.

The above structure refers to a specific enantiomer.

In some embodiments of any of the above methods or in some embodiments, the compound having the structure:

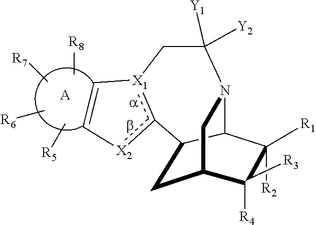

wherein
A is an aryl or heteroaryl;
X₁ is C or N;
X_{z} is N, O or S;
Y₁ is H, -(alkyl), -(alkenyl), -(alkynyl), -(cycloalkyl), -(haloalkyl), -(alkyl)-O-(alkyl) or -(alkyl)-(cycloalkyl);
Y₂ is H, -(alkyl), -(alkenyl), -(alkynyl), -(cycloalkyl), -(haloalkyl), -(alkyl)-O-(alkyl) or -(alkyl)-(cycloalkyl);
α and β are each present or absent and when present each is a bond,
   wherein either α or β is present,
   when α is present, then X₁ is C and X₂ is S or O, and
   when β is present, then X₁ is N and X₂ is N;
R₁, R₂, R₃ and R₄ are each independently —H, -(alkyl), -(alkenyl), -(alkynyl), -(haloalkyl), -(cycloalkyl), -(aryl), -(heteroaryl), -(heteroalkyl), -(hydroxyalkyl), -(alkyl)-(aryl), -(alkyl)- (heteroaryl), -(alkyl)-(cycloalkyl), -(alkyl)-OH, -(alkyl)-O-(alkyl), —OH, —NH₂, —OAc, —CO₂H, —CN, OCF₃, halogen, —CO₂—(C₂-C₁₂ alkyl), C(O)—NH₂, —C(O)—NH-(alkyl), C(O)—NH-(aryl), —O-alkyl, —O-alkenyl, —O-alkynyl, —O-aryl, —O-(heteroaryl), —NH-alkyl, —NH-alkenyl, —NH-alkynyl, —NH-aryl, —NH-(heteroaryl), —O—C(O) (alkyl) or —C(O)—N(alkyl)₂; and
R₅, R₆, R₇ and R₈ are each independently —H, halogen, —CN, —CF₃, —OCF₃, -(alkyl), -(alkenyl), -(alkynyl), -(haloalkyl), -(cycloalkyl), -(aryl), -(heteroaryl), -(heteroalkyl), -(hydroxyalkyl), —NH₂, —NH-(alkyl), —NH-(alkenyl), —NH-(alkynyl) —NH-(aryl), —NH-(heteroaryl), —OH, —OAc, —CO₂H, —CO₂-(alkyl), —O—C(O) (alkyl), —O-(alkyl), —O-(alkenyl), —O-(alkynyl), —O-(aryl), —O-(heteroaryl), —C(O)—NH₂, —C(O)—NH-(alkyl) or —C(O)—NH-(aryl),
   or a pharmaceutically acceptable salt or ester thereof.

The above structure refers to a specific enantiomer.

In some embodiments of any of the above methods or in some embodiments, the compound having the structure:

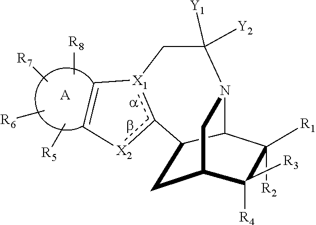

wherein
A is an aryl or heteroaryl;
X₁ is C or N;
X₂ is N, O or S;
Y₁ is H, -(alkyl), -(alkenyl) or -(alkynyl);
Y₂ is H, -(alkyl), -(alkenyl) or -(alkynyl);
α and β are each present or absent and when present each is a bond,
   wherein either α or β is present,
   when α is present, then X₁ is C and X₂ is S or O, and
   when β is present, then X₁ is N and X₂ is N;
R₁, R₂, R₃ and R₄ are each independently H, -(alkyl), -(alkenyl), -(alkynyl), -(aryl), -(heteroaryl), -(heteroalkyl), -(hydroxyalkyl), -(alkyl)-(aryl), -(alkyl)-(heteroaryl), -(alkyl)-OH, -(alkyl)- O-(alkyl), —OH, —NH₂, —CO₂H, —CO₂—(C₂-C₁₂ alkyl) or —C(O)—NH-(alkyl); and
R₅, R₆, R₇ and R₈ are each independently —H, halogen, —CN, —CF₃, —OCF₃, -(alkyl), -(alkenyl), -(alkynyl), -(aryl), -(heteroaryl), -(heteroalkyl), -(hydroxyalkyl), —NH₂, —NH-(alkyl), —NH-(alkenyl), —NH-(alkynyl) —NH-(aryl), —NH-(heteroaryl), —OH, —OAc, —CO₂H, —CO₂-(alkyl), —O—C(O) (alkyl), —O-(alkyl), —O-(alkenyl), —O-(alkynyl), —O-(aryl), —O-(heteroaryl), —C(O)—NH₂, —C(O)—NH-(alkyl) or C(O)—NH-(aryl),
   or a pharmaceutically acceptable salt or ester thereof.

The above structure refers to a specific enantiomer.

In some embodiments of any of the above methods or in some embodiments, the compound having the structure:

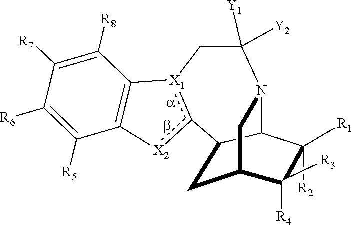

wherein
X₁ is C or N;
X₂ is N, O or S;
Y₁ is H, -(alkyl), -(alkenyl) or -(alkynyl);
Y₂ is H, -(alkyl), -(alkenyl) or -(alkynyl);
α and β are each present or absent and when present each is a bond,
   wherein either α or β is present,
   when α is present, then X₁ is C and X₂ is S or O, and
   when β is present, then X₁ is N and X₂ is N;
R₁, R₂, R₃ and R₄ are each independently H, -(alkyl), -(alkenyl), -(alkynyl), -(aryl), -(heteroaryl), -(heteroalkyl), -(hydroxyalkyl), -(alkyl)-(aryl), -(alkyl)-(heteroaryl), -(alkyl)-OH, -(alkyl)- O-(alkyl), —OH, —NH₂, —CO₂H, —CO₂—(C₂-C₁₂ alkyl) or —C(O)—NH-(alkyl); and
R₅, R₆, R₇, R₈ are each independently —H, halogen, —CN, —CF₃, —OCF₃, -(alkyl), -(alkenyl), -(alkynyl), -(aryl), -(heteroaryl), -(heteroalkyl), -(hydroxyalkyl), —NH₂, —NH-(alkyl), —NH-(alkenyl), —NH-(alkynyl) —NH-(aryl), —NH-(heteroaryl), —OH, —OAc, —CO₂H, —CO₂-(alkyl), —O—C(O) (alkyl), —O-(alkyl), —O-(alkenyl), —O-(alkynyl), —O-(aryl), —O-(heteroaryl), —C(O)—NH₂, —C(O)—NH-(alkyl) or C(O)—NH-(aryl),
   or a pharmaceutically acceptable salt or ester thereof.

The above structure refers to a specific enantiomer.

In some embodiments of any of the above methods or in some embodiments, the compound having the structure:

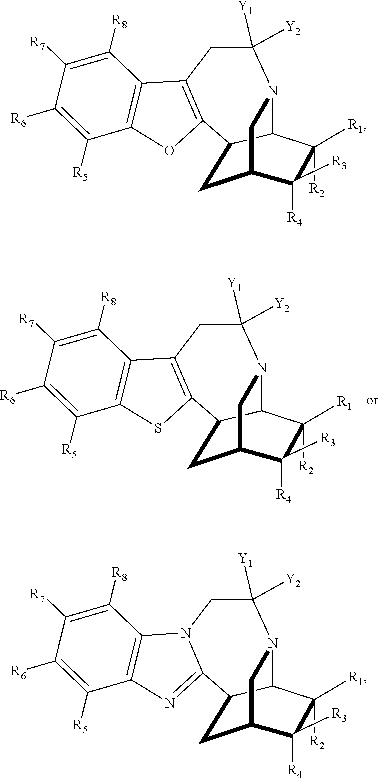

or a pharmaceutically acceptable salt or ester thereof.

The above structures refer to a specific enantiomer.

In some embodiments of any of the above methods or in some embodiments, wherein in the compound
R₁ is —H and R₂ is -(alkyl), -(alkenyl), -(alkynyl), -(aryl), -(heteroaryl), -(alkyl)-OH, -(alkyl)-(aryl), -(alkyl)-O-(alkyl), —OH, —NH₂, —CO₂-(alkyl) or —C(O)—NH-(alkyl).

In some embodiments of any of the above methods or in some embodiments, wherein in the compound
R₂ is —H and R₁ is -(alkyl), -(alkenyl), -(alkynyl), -(aryl), -(heteroaryl), -(alkyl)-OH, -(alkyl)-(aryl), -(alkyl)-O-(alkyl), —OH, —NH₂, —CO₂-(alkyl) or —C(O)—NH-(alkyl).

In some embodiments of any of the above methods or in some embodiments, wherein in the compound
R₃ is —H and R₄ is -(alkyl), -(alkenyl), -(alkynyl), -(aryl), -(heteroaryl), -(alkyl)-OH, -(alkyl)-(aryl), -(alkyl)-O-(alkyl), —OH, —NH₂, —CO₂-(alkyl) or —C(O)—NH-(alkyl).

In some embodiments of any of the above methods or in some embodiments, wherein in the compound
R₄ is —H and R₃ is -(alkyl), -(alkenyl), -(alkynyl), -(aryl), -(heteroaryl), -(alkyl)-OH, -(alkyl)-(aryl), -(alkyl)-O-(alkyl), —OH, —NH₂, —CO₂-(alkyl) or —C(O)—NH-(alkyl).

In some embodiments of any of the above methods or in some embodiments, wherein in the compound
R₁ and R₂ are each —H.

In some embodiments of any of the above methods or in some embodiments, wherein in the compound
R₃ and R₄ are each —H.

In some embodiments of any of the above methods or in some embodiments, wherein in the compound
R₁, R₂, R₃ and R₄ are each —H.

In some embodiments of any of the above methods or in some embodiments, wherein in the compound
R₅, R₆, R₇ and R₈ are each —H.

In some embodiments of any of the above methods or in some embodiments, wherein in the compound
R₅, R₆ and R₇ are each —H and R₈ is halogen, —CN, —CF₃, —OCF₃, -(alkyl), -(alkenyl), -(alkynyl), -(aryl), -(heteroaryl), —NH₂, —NH-(alkyl), —NH-(alkenyl), —NH-(alkynyl) —NH-(aryl), —NH-(heteroaryl), —OH, —OAc, —O—C(O) (alkyl), —O-(alkyl), —O-(alkenyl), —O-(alkynyl), —O-(aryl), —O-(heteroaryl), —C(O)—NH₂, —C(O)—NH-(alkyl) or —C(O)—NH-(aryl).

In some embodiments of any of the above methods or in some embodiments, wherein in the compound
R₅, R₆ and R₈ are each —H and R₇ is halogen, —CN, —CF₃, —OCF₃, -(alkyl), -(alkenyl), -(alkynyl), -(aryl), -(heteroaryl), —NH₂, —NH-(alkyl), —NH-(alkenyl), —NH-(alkynyl) —NH-(aryl), —NH-(heteroaryl), —OH, —OAc, —O—C(O) (alkyl), —O-(alkyl), —O-(alkenyl), —O-(alkynyl), —O-(aryl), —O-(heteroaryl), —C(O)—NH₂, —C(O)—NH-(alkyl) or —C(O)—NH-(aryl).

In some embodiments of any of the above methods or in some embodiments, wherein in the compound
R₅, R₇ and R₈ are each —H and R₆ is halogen, —CN, —CF₃, —OCF₃, -(alkyl), -(alkenyl), -(alkynyl), -(aryl), -(heteroaryl), —NH₂, —NH-(alkyl), —NH-(alkenyl), —NH-(alkynyl) —NH-(aryl), —NH-(heteroaryl), —OH, —OAc, —O—C(O) (alkyl), —O-(alkyl), —O-(alkenyl), —O-(alkynyl), —O-(aryl), —O-(heteroaryl), —C(O)—NH₂, —C(O)—NH-(alkyl) or —C(O)—NH-(aryl).

In some embodiments of any of the above methods or in some embodiments, wherein in the compound
R₆, R₇ and R₈ are each —H and R₅ is halogen, —CN, —CF₃, —OCF₃, -(alkyl), -(alkenyl), -(alkynyl), -(aryl), -(heteroaryl), —NH₂, —NH-(alkyl), —NH-(alkenyl), —NH-(alkynyl) —NH-(aryl), —NH-(heteroaryl), —OH, —OAc, —O—C(O) (alkyl), —O-(alkyl), —O-(alkenyl), —O-(alkynyl), —O-(aryl), —O-(heteroaryl), —C(O)—NH₂, —C(O)—NH-(alkyl) or —C(O)—NH-(aryl).

In some embodiments of any of the above methods or in some embodiments, wherein in the compound
R₅ and R₈ are each —H and R₆ and R₇ are each independently halogen, —CN, —CF₃, —OCF₃, -(alkyl), -(alkenyl), -(alkynyl), -(aryl), -(heteroaryl), —NH₂, —NH-(alkyl), —NH-(alkenyl), —NH-(alkynyl) —NH-(aryl), —NH-(heteroaryl), —OH, —OAc, —O—C(O) (alkyl), —O-(alkyl), —O-(alkenyl), —O-(alkynyl), —O-(aryl), —O-(heteroaryl), —C(O)—NH₂, —C(O)—NH-(alkyl) or —C(O)—NH-(aryl).

In some embodiments of any of the above methods or in some embodiments, the compound having the structure:

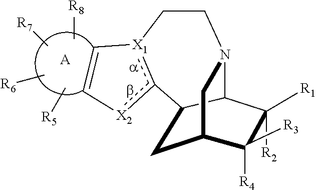

wherein
A is phenyl;
X₁ is C or N;
X₂ is N, O or S;
α and β are each present or absent and when present each is a bond,
   wherein either α or β is present,
   when α is present, then X₁ is C and X₂ is S or O, and
   when β is present, then X₁ is N and X₂ is N;
R₁, R₂, R₃ and R₄ are each independently H, -(alkyl), -(aryl), -(alkyl)-OH, -(alkyl)-(aryl), or -(alkyl)-O-(alkyl), R₅, R₆, R₇ and R₈ are each independently —H, halogen, —OH, —O-(alkyl), —C(O)—NH₂, —C(O)—NH-(alkyl) or —C(O)—NH-(aryl),
   or a pharmaceutically acceptable salt or ester thereof.

The above structure refers to a specific enantiomer.

In some embodiments of any of the above methods or in some embodiments, the compound having the structure:

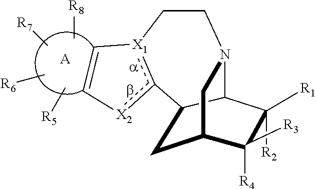

wherein
A is phenyl;
X₁ is C or N;
X₂ is N, O or S;
α and β are each present or absent and when present each is a bond,
   wherein either α or β is present,
   when α is present, then X₁ is C and X₂ is S or O, and
   when β is present, then X₁ is N and X₂ is N;
R₁, R₂, R₃ and R₄ are each independently H, -(alkyl), -(aryl), -(alkyl)-OH, -(alkyl)-(aryl) or -(alkyl)-O-(alkyl), R₅, R₆, R₇ and R₈ are each independently —H, -(alkyl), halogen, —OH, —O-(alkyl), —C(O)—NH₂, —C(O)—NH-(alkyl) or —C(O)—NH-(aryl),
   or a pharmaceutically acceptable salt or ester thereof.

The above structure refers to a specific enantiomer.

In some embodiments of any of the above methods or in some embodiments, wherein in the compound
Y₁ is H or -(alkyl); and
Y₂ is H or -(alkyl).

In some embodiments of any of the above methods or in some embodiments, wherein in the compound
Y₁ is H, —CH₃, —CH₂CH₃ or —CH₂CH₂CH₃; and
Y₂ is H, —CH₃, —CH₂CH₃ or —CH₂CH₂CH₃.

In some embodiments of any of the above methods or in some embodiments, the compound having the structure:

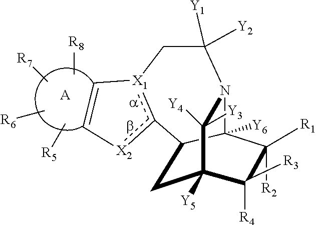

wherein
A is a ring structure, with or without substitution;
X₁ is C or N;
X₂ is N, O or S;
Y₁ is H, -(alkyl), -(alkenyl), -(alkynyl), -(cycloalkyl), -(haloalkyl), -(alkyl)-O-(alkyl) or -(alkyl)-(cycloalkyl);
Y₂ is H, -(alkyl), -(alkenyl), -(alkynyl), -(cycloalkyl), -(haloalkyl), -(alkyl)-O-(alkyl) or -(alkyl)-(cycloalkyl);
Y₃ is H, -(alkyl), -(alkenyl), -(alkynyl), -(cycloalkyl), -(haloalkyl), -(alkyl)-O-(alkyl) or -(alkyl)-(cycloalkyl);
Y₄ is H, -(alkyl), -(alkenyl), -(alkynyl), -(cycloalkyl), -(haloalkyl), -(alkyl)-O-(alkyl) or -(alkyl)-(cycloalkyl);
Y₅ is H, -(alkyl), -(alkenyl), -(alkynyl), -(cycloalkyl), -(haloalkyl), -(alkyl)-O-(alkyl) or -(alkyl)-(cycloalkyl);
Y₆ is H, -(alkyl), -(alkenyl), -(alkynyl), -(cycloalkyl), -(haloalkyl), -(alkyl)-O-(alkyl) or -(alkyl)-(cycloalkyl);
α and β are each present or absent and when present each is a bond,
   wherein either α or β is present, and
   when α is present, then X₁ is C and X₂ is S or O, or
   when β is present, then X, is N and X₂ is N; and
R₁, R₂, R₃ and R₄ are each independently H, -(alkyl), -(alkenyl), -(alkynyl), -(aryl), -(heteroaryl), -(heteroalkyl), -(hydroxyalkyl), -(alkyl)-(aryl), -(alkyl)-(heteroaryl), -(alkyl)-OH, -(alkyl)- O-(alkyl), —OH, —NH₂, —CO₂H, —CO₂—(C₂-C₁₂ alkyl) or —C(O)—NH-(alkyl),
   or a pharmaceutically acceptable salt or ester thereof.

The above structure refers to a specific enantiomer.

In some embodiments of any of the above methods or in some embodiments, the compound having the structure:

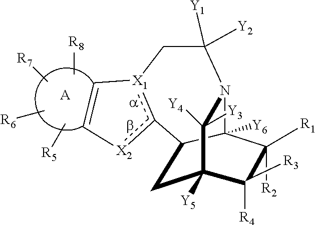

wherein
A is a ring structure, with or without substitution;
X₁ is C or N;
X₂ is N, O or S;
Y₁ is H, -(alkyl), -(alkenyl), -(alkynyl), -(cycloalkyl) or -(alkyl)-(cycloalkyl);
Y₂ is H, -(alkyl), -(alkenyl), -(alkynyl), -(cycloalkyl) or -(alkyl)-(cycloalkyl);
Y₃ is H, -(alkyl), -(alkenyl), -(alkynyl), -(cycloalkyl) or -(alkyl)-(cycloalkyl);
Y₄ is H, -(alkyl), -(alkenyl), -(alkynyl), -(cycloalkyl) or -(alkyl)-(cycloalkyl);
Y₅ is H, -(alkyl), -(alkenyl), -(alkynyl), -(cycloalkyl) or -(alkyl)-(cycloalkyl);
Y₆ is H, -(alkyl), -(alkenyl), -(alkynyl), -(cycloalkyl) or -(alkyl)-(cycloalkyl);
α and β are each present or absent and when present each is a bond,
   wherein either α or β is present, and
   when α is present, then X₁ is C and X₂ is S or O, or
   when β is present, then X₁ is N and X₂ is N; and
R₁, R₂, R₃ and R₄ are each independently H, -(alkyl), -(alkenyl), -(alkynyl), -(aryl), -(heteroaryl), -(heteroalkyl), -(hydroxyalkyl), -(alkyl)-(aryl), -(alkyl)-(heteroaryl), -(alkyl)-OH, -(alkyl)- O-(alkyl), —OH, —NH₂, —CO₂H, —CO₂—(C₂-C₁₂ alkyl) or —C(O)—NH-(alkyl),
   or a pharmaceutically acceptable salt or ester thereof.

In some embodiments of any of the above methods or in some embodiments, the compound having the structure:

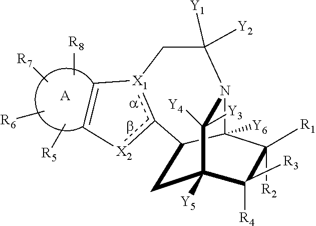

wherein
A is an aryl or heteroaryl;
X₁ is C or N;
X₂ is N, O or S;
Y₁ is H, -(alkyl), -(alkenyl), -(alkynyl), -(cycloalkyl) or -(alkyl)-(cycloalkyl);
Y₂ is H, -(alkyl), -(alkenyl), -(alkynyl), -(cycloalkyl) or -(alkyl)-(cycloalkyl);
Y₃ is H, -(alkyl), -(alkenyl), -(alkynyl), -(cycloalkyl) or -(alkyl)-(cycloalkyl);
Y₄ is H, -(alkyl), -(alkenyl), -(alkynyl), -(cycloalkyl) or -(alkyl)-(cycloalkyl);
Y₅ is H, -(alkyl), -(alkenyl), -(alkynyl), -(cycloalkyl) or -(alkyl)-(cycloalkyl);
Y₆ is H, -(alkyl), -(alkenyl), -(alkynyl), -(cycloalkyl) or -(alkyl)-(cycloalkyl);
α and β are each present or absent and when present each is a bond,
   wherein either α or β is present,
   when α is present, then X₁ is C and X₂ is S or O, and
   when β is present, then X₁ is N and X₂ is N;
R₁, R₂, R₃ and R₄ are each independently H, -(alkyl), -(alkenyl), -(alkynyl), -(aryl), -(heteroaryl), -(heteroalkyl), -(hydroxyalkyl), -(alkyl)-(aryl), -(alkyl)-(heteroaryl), -(alkyl)-OH, -(alkyl)- O-(alkyl), —OH, —NH₂, —CO₂H, —CO₂—(C₂-C₁₂ alkyl) or —C(O)—NH-(alkyl); and
R₅, R₆, R₇, R₈ are each independently —H, halogen, —CN, —CF₃, —OCF₃, -(alkyl), -(alkenyl), -(alkynyl), -(aryl), -(heteroaryl), -(heteroalkyl), -(hydroxyalkyl), —NH₂, —NH-(alkyl), —NH-(alkenyl), —NH-(alkynyl) —NH-(aryl), —NH-(heteroaryl), —OH, —OAc, —CO₂H, —CO₂-(alkyl), —O—C(O) (alkyl), —O-(alkyl), —O-(alkenyl), —O-(alkynyl), —O-(aryl), —O-(heteroaryl), —C(O)—NH₂, —C(O)—NH-(alkyl) or —C(O)—NH-(aryl),
   or a pharmaceutically acceptable salt or ester thereof.

In some embodiments of any of the above methods or in some embodiments, the compound having the structure:

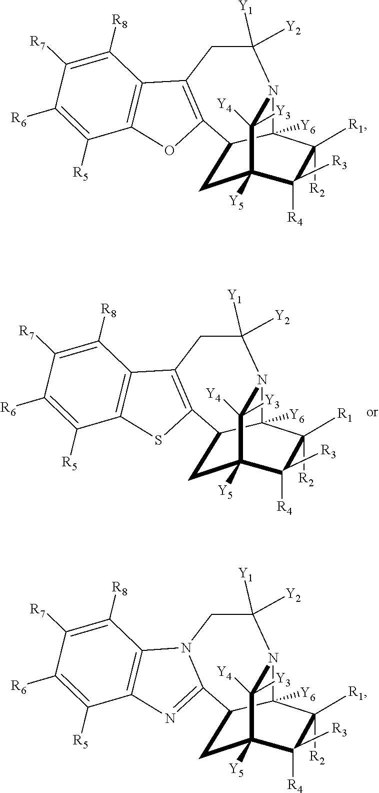

or a pharmaceutically acceptable salt or ester thereof.

The above structures refer to a specific enantiomer.

In some embodiments of any of the above methods or in some embodiments, the compound having the structure:

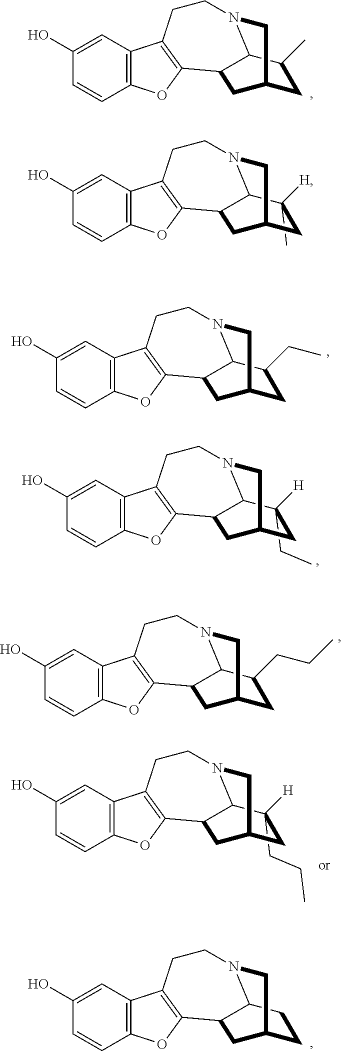

or a pharmaceutically acceptable salt or ester thereof.

The above structures refer to a specific enantiomer.

In some embodiments of any of the above methods or in some embodiments, the compound having the structure:

or a pharmaceutically acceptable salt or ester thereof.

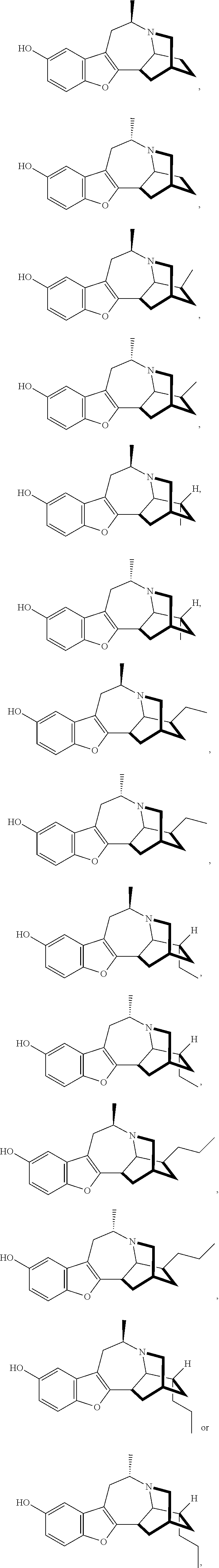

The above structures refer to a specific enantiomer.

In some embodiments of any of the above methods or in some embodiments, the compound having the structure:

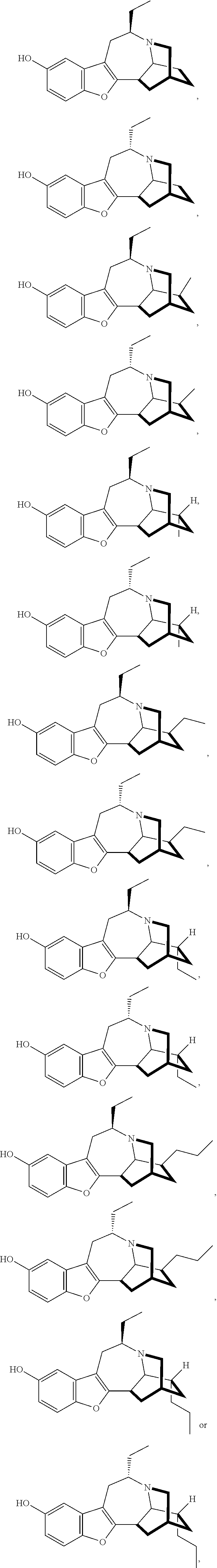

or a pharmaceutically acceptable salt or ester thereof.

The above structures refer to a specific enantiomer.

In some embodiments of any of the above methods or in some embodiments, the compound having the structure:

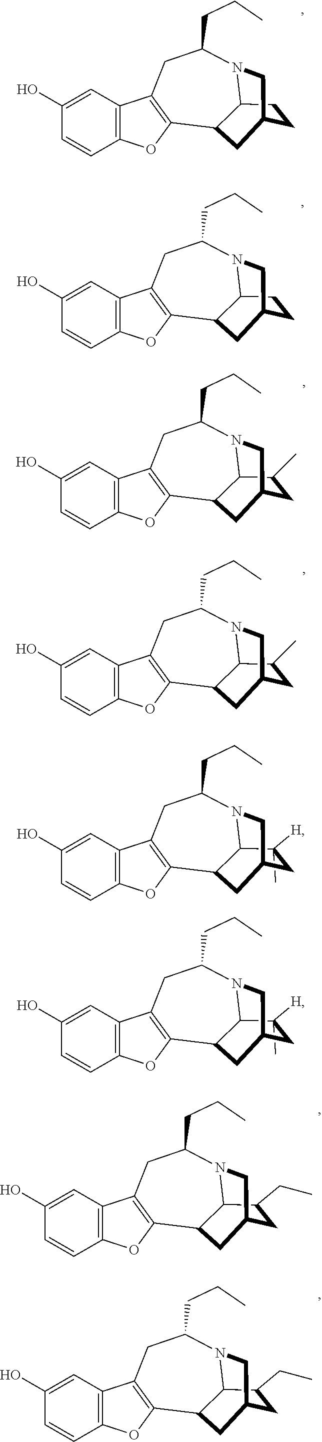

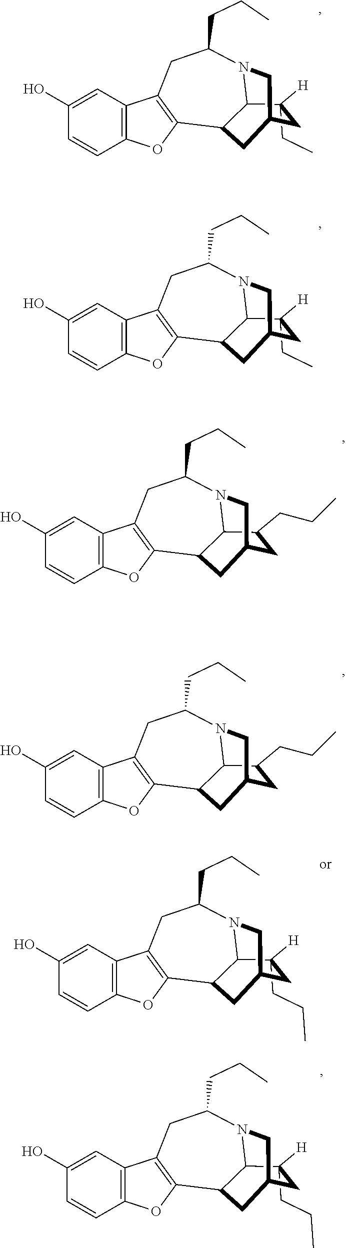

or a pharmaceutically acceptable salt or ester thereof.

The above structures refer to a specific enantiomer.

In some embodiments of any of the above methods or in some embodiments, the compound having the structure:

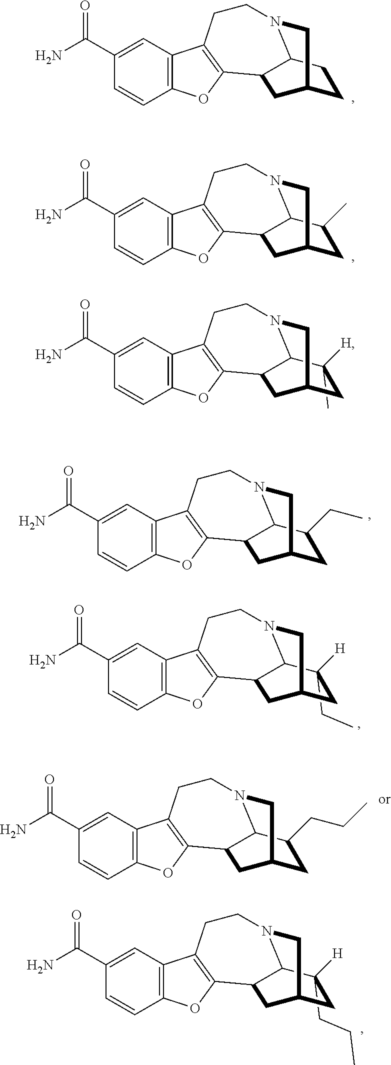

or a pharmaceutically acceptable salt or ester thereof.

The above structures refer to a specific enantiomer.

In some embodiments of any of the above methods or in some embodiments, the compound having the structure:

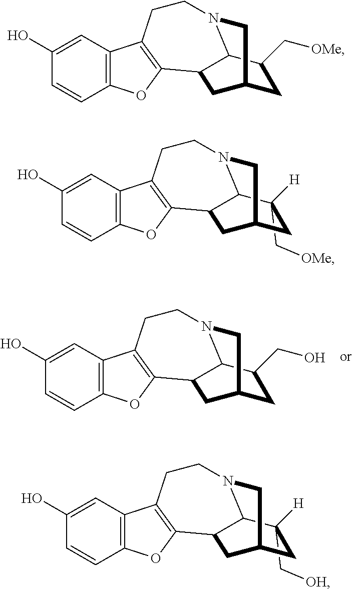

or a pharmaceutically acceptable salt or ester thereof.

The above structures refer to a specific enantiomer.

The present invention provides a pharmaceutical composition comprising the compound of the present invention and a pharmaceutically acceptable carrier.

The present invention provides a method of activating mu-opioid receptor, delta-opioid receptor and/or kappa-opioid receptor comprising contacting the mu-opioid receptor, delta-opioid receptor and/or kappa-opioid receptor with the compound of the present invention.

The present invention provides a method of inhibiting mu-opioid receptor, delta-opioid receptor and/or kappa-opioid receptor comprising contacting the mu-opioid receptor, delta-opioid receptor and/or kappa-opioid receptor with the compound of the present invention.

The present invention provides a method of inhibiting serotonin transporter (SERT) comprising contacting the serotonin transporter (SERT) with the compound of the present invention.

The present invention provides a method of treating a subject afflicted with depression, major depression, pain, a mood disorder, anxiety disorder, obsessive-compulsive disorder (OCD) or stress disorder comprising administering to the subject the compound of the present invention, or the composition of the present invention comprising an effective amount of the compound, so as to thereby treat the subject afflicted with depression, major depression, pain, anxiety disorder, obsessive-compulsive disorder (OCD) or stress disorder.

In an embodiment, the pain is acute. In another embodiment, the pain is chronic.

The present invention provides a method of altering the psychological state of a subject comprising administering to the subject the compound of the present invention, or the composition of the present invention comprising an effective amount of the compound, so as to thereby alter the psychological state of the subject.

The present invention provides a method of enhancing the effect of psychotherapy in a subject comprising administering to the subject the compound of the present invention, or the composition of the present invention comprising an effective amount of the compound, so as to thereby enhance the effect of the psychotherapy in the subject.

The present invention provides a method of treating a subject afflicted with Parkinson's disease, or traumatic brain injury comprising administering to the subject the compound of the present invention, or the composition of of the present invention comprising an effective amount of the compound, so as to thereby treat the subject afflicted with Parkinson's disease or traumatic brain injury.

The present invention provides a method of treating a subject afflicted with a headache or a migraine comprising administering to the subject the compound of the present invention, or the composition of the present invention comprising an effective amount of the compound, so as to thereby treat the subject afflicted with the headache or the migraine.

The present invention provides a method of treating a subject afflicted with a substance use disorder comprising administering to the subject the compound of the present invention, or the composition of the present invention comprising an effective amount of the compound, so as to thereby treat the subject afflicted with the substance use disorder.

In some embodiments, wherein the substance use disorder is opioid use disorder, alcohol use disorder or stimulant use disorder including nicotine use disorder.

In some embodiments, wherein the substance is an opioid.

In some embodiments, wherein the opioid is morphine, hydromorphone, oxymorphone, codeine, dihydrocodeine, hydrocodone, oxycodone, nalbuphine, butorphanol, etorphine, dihydroetorphine, levorphanol, metazocine, pentazocine, meptazinol, meperidine (pethidine), buprenorphine, methadone, tramadol, tapentadol, mitragynine, 3-deutero-mitragynine, 7-hydroxymitragynine, 3-deutero-7-hydroxymitragynine, mitragynine pseudoindoxyl or tianeptine.

In some embodiments, wherein the opioid is fentanyl, sufentanil or alfentanil.

In some embodiments, wherein the opioid is a derivative of fentanyl.

Derivatives of fentanyl include, but are not limited to, N-(1-(2-phenylethyl)-4-piperidinyl)-N-phenylfuran-2-carboxamide (furanylfentanyl); N-(3-methyl-1-phenethyl-4-piperidyl)-N-phenyl-propanamide (3-methylfentanyl); N-phenyl-N-[1-(2-phenylethyl)piperidin-4-yl]pentanamide (valerylfentanyl); N-(1-(2-phenylethyl)-4-piperidinyl)-N-phenylbutyramide (butyrylfentanyl); N-{1-[2-hydroxy-2-(thiophen-2-yl)ethyl]piperidin-4-yl}-N-phenylpropanamide (β-Hydroxythiofentanyl); N-Phenyl-N-[1-(2-phenylethyl)piperidin-4-yl]prop-2-enamide (acrylfentanyl); and 4-((1-oxopropyl)-phenylamino)-1-(2-phenylethyl)-4-piperidinecarboxylic acid methyl ester (carfentanil).

In some embodiments, wherein the stimulant is cocaine, amphetamine, methamphetamine or cathinone.

In some embodiments, wherein the stimulant is a derivative of cathinone.

Derivatives of cathinone include, but are not limited to, Amfepramone (diethylpropion), Mephedrone (4-methylmethcathinone, 4-MMC), Methylone (Pk-MDMA, 3,4-methylenedioxy-N-methylcathinone), Methcathinone (ephedrone), MDPV (3,4-methylenedioxypyrovalerone), Methedrone (Rk-PMMA, 4-methoxymethcathinone).

In some embodiments, wherein the stimulant is nicotine.

The present invention provides a method of treating a subject afflicted with opioid withdrawal symptoms comprising administering to the subject the compound of the present invention, or the composition of the present invention comprising an effective amount of the compound, so as to thereby treat the subject afflicted with the opioid withdrawal symptoms.

The present invention provides a method of treating a subject afflicted with a symptom of substance use disorder comprising administering to the subject the compound of the present invention, or the composition of the present invention comprising an effective amount of the compound, so as to thereby treat the subject afflicted with the symptom of substance use disorder.

In an embodiment, wherein a symptom of substance use disorder is opioid withdrawal. In another embodiment, wherein a symptom of substance use disorder is mitigation of relapse to opioid use or SUD.

In an embodiment, wherein a symptom of substance use disorder is hyperalgesia or allodynia.

In an embodiment, wherein a symptom of substance use disorder is hyperalgesia. In an embodiment, wherein a symptom of substance use disorder is allodynia.

In some embodiments, wherein the risk of relapse to the use of opioids, alcohol or stimulants is reduced.

In some embodiments of any of the above methods, wherein self-administration of an opioid, alcohol or stimulant is reduced. In an embodiment of any of the above methods, wherein self-administration of an opioid is reduced. In an embodiment of any of the above methods, wherein self-administration of alcohol is reduced. In an embodiment of any of the above methods, wherein self-administration of a stimulant is reduced.

In some embodiments, wherein the treating is effective for an extended time.

In some embodiments, wherein the time is 1-5 days. In an embodiment, wherein the time is 1 day. In an embodiment, wherein the time is 2 days. In an embodiment, wherein the time is 3 days. In an embodiment, wherein the time is 4 days. In an embodiment, wherein the time is 5 days.

In some embodiments, wherein the time is 1-5 weeks. In an embodiment, wherein the time is 1 week. In an embodiment, wherein the time is 2 weeks. In an embodiment, wherein the time is 3 weeks. In an embodiment, wherein the time is 4 weeks. In an embodiment, wherein the time is 5 weeks.

In some embodiments, wherein the effective amount of the compound administered to the subject without inducing cardiotoxicity.

In some embodiments, wherein the effective amount of the compound administered to the subject without inducing QT interval prolongation.

In some embodiments, wherein the effective amount of the compound administered to the subject without inducing cardiac arrhythmia.

In an embodiment, wherein the subject is a mammal.

In an embodiment, wherein the mammal is a human.

In some embodiments, wherein the effective amount of 10-500 mg of the compound is administered to the subject.

In some embodiments, a method of treating a subject afflicted with depression, major depression, pain, a mood disorder, anxiety disorder, obsessive-compulsive disorder (OCD) or stress disorder, comprising administering to the subject the composition of the present invention comprising an effective amount of the compound so as to thereby treat the subject afflicted with depression, major depression, pain, anxiety disorder, obsessive-compulsive disorder (OCD) or stress disorder.

In an embodiment, the pain is acute. In another embodiment, the pain is chronic.

In some embodiments, a method of altering the psychological state of a subject comprising administering to the subject the composition of the present invention comprising an effective amount of the compound so as to thereby alter the psychological state of the subject.

In some embodiments, a method of enhancing the effect of psychotherapy comprising administering to the subject the composition of the present invention comprising an effective amount of the compound so as to thereby enhance the effect of the psychotherapy in the subject.

In some embodiments, a method of treating a subject afflicted with a headache or a migraine comprising administering to the subject the composition of the present invention comprising an effective amount of the compound, so as to thereby treat the subject afflicted with the headache or the migraine.

In some embodiments, a method of treating a subject afflicted with a substance use disorder comprising administering to the subject the composition of the present invention comprising an effective amount of the compound, so as to thereby treat the subject afflicted with the substance use disorder.

In some embodiments, wherein the substance use disorder is opioid use disorder, alcohol use disorder or stimulant use disorder including nicotine/tobacco use disorder/tobacco smoking.

In some embodiments, a method of treating a subject afflicted with opioid withdrawal symptoms comprising administering to the subject the composition of the present invention comprising an effective amount of the compound, so as to thereby treat the subject afflicted with the opioid withdrawal symptoms.

In an embodiment, wherein a symptom of substance use disorder is opioid withdrawal. In another embodiment, wherein a symptom of substance use disorder is mitigation of relapse to opioid use or SUD.

In some embodiments, a method of reducing opioid cravings in a subject afflicted with an opioid use disorder comprising administering to the subject the composition of the present invention comprising an effective amount of the compound so as to reduce the subject's opioid cravings.

In some embodiments, wherein the substance use disorder is opioid use disorder, alcohol use disorder, stimulant use disorder or polydrug use disorder.

In some embodiments, wherein the stimulant use disorder is nicotine use disorder.

In some embodiments, a method of treating a subject afflicted with opioid use disorder comprising administering to the subject an effective amount of mu-opioid receptor agonist and the composition of the present invention comprising an effective amount of the compound so as to treat the subject afflicted with the opioid use disorder.

In some embodiments, a method of treating a subject afflicted with alcohol withdrawal symptoms or stimulant withdrawal symptoms comprising administering to the subject the composition of the present invention comprising an effective amount of the compound so as to treat the subject afflicted with the opioid withdrawal symptoms.

In some embodiments, a method of treating a subject afflicted with traumatic brain injury (TBI) comprising administering to the subject the composition of the present invention comprising an effective amount of the compound so as to treat the subject afflicted with the traumatic brain injury (TBI).

In some embodiments, a method of treating a subject afflicted with Parkinson's disease comprising administering to the subject the composition of the present invention comprising an effective amount of the compound so as to treat the subject afflicted with the Parkinson's disease.

In some embodiments, a method of treating a subject afflicted with a headache or a migraine comprising administering to the subject the composition of the present invention comprising an effective amount of the compound so as to treat the subject afflicted with a headache or a migraine.

In some embodiments, a method of treating a subject afflicted with opioid use disorder comprising administering to the subject an effective amount of mu-opioid receptor agonist and the composition of the present invention comprising an effective amount of the compound so as to treat the subject afflicted with the opioid use disorder.

In some embodiments, a method of treating a subject afflicted with opioid use disorder comprising administering to the subject an effective amount of an opioid or opiate and the composition of the present invention comprising an effective amount of the compound so as to treat the subject afflicted with the opioid use disorder.

In some embodiments, a method of treating a subject afflicted with opioid use disorder comprising administering to the subject an effective amount of morphine, hydromorphone, oxymorphone, codeine, dihydrocodeine, hydrocodone, oxycodone, nalbuphine, butorphanol, etorphine, dihydroetorphine, levorphanol, metazocine, pentazocine, meptazinol, meperidine (pethidine), fentanyl, sufentanil, alfentanil, buprenorphine, methadone, tramadol, tapentadol, mitragynine, 3-deutero-mitragynine, 7-hydroxymitragynine, 3-deutero-7-hydroxymitragynine, mitragynine pseudoindoxyl, tianeptine, 7-((3-bromo-6-methyl-5,5-dioxido-6,11-dihydrodibenzo[c,f][1,2]thiazepin-11-yl)amino)heptanoic acid, 7-((3-iodo-6-methyl-5,5-dioxido-6,11-dihydrodibenzo[c,f] [1,2] thiazepin-11-yl)amino)heptanoic acid, 5-((3-bromo-6-methyl-5,5-dioxido-6,11-dihydrodibenzo[c,f][1,2]thiazepin-11-yl)amino)pentanoic acid or 5-((3-iodo-6-methyl-5,5-dioxido-6,11-dihydrodibenzo[c,f] [1,2]thiazepin-11-yl)amino)pentanoic acid and the composition of the present invention comprising an effective amount of the compound so as to treat the subject afflicted with the opioid use disorder.

In some embodiments, a method of treating a subject afflicted with opioid use disorder or opioid withdrawal symptoms comprising administering to the subject an effective amount of naloxone or methylnaltrexone and the composition of the present invention comprising an effective amount of the compound so as to thereby treat the subject afflicted with the opioid use disorder or opioid withdrawal symptoms.

In some embodiments, a method of treating a subject afflicted with substance use disorder or opioid withdrawal symptoms comprising administering to the subject an effective amount of Suboxone or Naltrexone and the composition of the present invention comprising an effective amount of the compound so as to thereby treat the subject afflicted with the opioid use disorder or opioid withdrawal symptoms.

The present invention also provides a pharmaceutical composition comprising the compound of the present application and a pharmaceutically acceptable carrier.

The present invention also provides a method of activating mu-opioid receptor comprising contacting the mu-opioid receptor with the compound of the present application.

The present invention also provides a method of activating delta-opioid receptor comprising contacting the delta-opioid receptor with the compound of the present application.

The present invention also provides a method of activating kappa-opioid receptor comprising contacting the kappa-opioid receptor with the compound of the present application.

The present invention also provides a method of inhibiting mu-opioid receptor comprising contacting the mu-opioid receptor with the compound of the present application.

The present invention also provides a method of inhibiting delta-opioid receptor comprising contacting the delta-opioid receptor with the compound of the present application.

The present invention also provides a method of inhibiting kappa-opioid receptor comprising contacting the kappa-opioid receptor with the compound of the present application.

The present invention also provides a method of treating a subject afflicted with depression or major depression comprising administering an effective amount of the compound of the present application to the subject so as to treat the depression or major depression.

The present invention also provides a method of treating a subject afflicted with pain comprising administering an effective amount of the compound of the present application to the subject so as to treat the pain.

The present invention also provides a method of treating a subject afflicted with an anxiety disorder comprising administering an effective amount of the compound of the present application to the subject so as to treat the anxiety disorder.

The present invention also provides a method of treating a subject afflicted with obsessive-compulsive disorder (OCD) comprising administering an effective amount of the compound of the present application to the subject so as to treat the obsessive-compulsive disorder (OCD).

The present invention also provides a method of treating a subject afflicted with a stress disorder comprising administering an effective amount of the compound of the present application to the subject so as to treat the stress disorder.

In some embodiments of any of the above methods, the compound activates mu-opioid, delta-opioid, or kappa-opioid receptors or any combination thereof in the subject.

In some embodiments of any of the above methods, the compound is an agonist of mu-opioid, delta-opioid, or kappa-opioid receptors or any combination thereof in the subject.

In some embodiments of any of the above methods, the compound inhibits mu-opioid, delta-opioid, or kappa-opioid receptors or any combination thereof in the subject.

In some embodiments of any of the above methods, the compound is an antagonist of mu-opioid, delta-opioid, or kappa-opioid receptors or any combination thereof in the subject.

In some embodiments of any of the above methods, the compound inhibits serotonin transporter (SERT).

In some embodiments of any of the above methods, the compound inhibits acetylcholine nicotinic receptors. In a further embodiment of any of the above methods, the compound inhibits α3β4 acetylcholine nicotinic receptor.

The present invention provides a pharmaceutical composition comprising the compound of the present invention and a pharmaceutically acceptable carrier.

The present invention provides a method of activating mu-opioid receptor comprising contacting the mu-opioid receptor with the compound of the present invention.

The present invention provides a method of activating delta-opioid receptor comprising contacting the delta-opioid receptor with the compound of the present invention.

The present invention provides a method of activating kappa-opioid receptor comprising contacting the kappa-opioid receptor with the compound of the present invention.

The present invention provides a method of inhibiting serotonin transporter (SERT) comprising contacting the serotonin transporter (SERT) with the compound of the present invention.

The present invention provides a method of treating a subject afflicted with depression or major depression comprising administering an effective amount of the compound of the present invention to the subject so as to treat the depression or major depression.

The present invention provides a method of treating a subject afflicted with pain comprising administering an effective amount of the compound of the present invention to the subject so as to treat the pain.

The present invention provides a method of treating a subject afflicted with anxiety comprising administering an effective amount of the compound of the present invention to the subject so as to treat the anxiety.

The present invention provides a method of treating a subject afflicted with stress related disorders comprising administering an effective amount of the compound of the present invention to the subject so as to treat the stress related disorder.

In some embodiments, the mu-opioid, delta-opioid or kappa-opioid receptors are in a human subject.

In some embodiments, the serotonin transporters (SERT) are in a human subject.

In some embodiments, the stress disorder is post-traumatic stress disorder (PTSD) or acute stress disorder.

In some embodiments, the anxiety disorder is panic disorder, social anxiety disorder, generalized anxiety disorder or a specific phobia.

In some embodiments, a pharmaceutically acceptable salt of any of the above compounds of the present invention.

In some embodiments, any of the above compounds for use in activating the mu-opioid receptor, delta-opioid receptor and/or kappa-opioid receptor.

In some embodiments, any of the above compounds for use in inhibiting the mu-opioid receptor, delta-opioid receptor and/or kappa-opioid receptor.

In some embodiments, any of the above compounds for use in inhibiting the serotonin transporter (SERT).

In some embodiments, any of the above compounds for use in treating a subject afflicted with depression, major depression, pain, anxiety disorder, obsessive-compulsive disorder (OCD) or stress disorder.

In some embodiments, any of the above compounds for use in treating depression, major depression, pain, anxiety disorder, obsessive-compulsive disorder (OCD) or stress disorder.

In some embodiments, use of any of the above compounds for activating the mu-opioid receptor, delta-opioid receptor and/or kappa-opioid receptor.

In some embodiments, use of any of the above compounds for inhibiting the mu-opioid receptor, delta-opioid receptor and/or kappa-opioid receptor.

In some embodiments, use of any of the above compounds for treating a subject afflicted with depression, major depression, pain, anxiety disorder, obsessive-compulsive disorder (OCD) or stress disorder.

In some embodiments, use of any of the above compounds for treating depression, major depression, pain, anxiety disorder, obsessive-compulsive disorder (OCD) or stress disorder.

In some embodiments, a pharmaceutical composition comprising any of the above compounds for treating a subject afflicted with depression, major depression, pain, anxiety disorder, obsessive-compulsive disorder (OCD) or stress disorder.

In some embodiments, a pharmaceutical composition comprising any of the above compounds for treating depression, major depression, pain, anxiety disorder, obsessive-compulsive disorder (OCD) or stress disorder.

Opioid use disorder (OUD) involves, but is not limited to, misuse of opioid medications or use of illicitly obtained opioids. The Diagnostic and Statistical Manual of Mental Disorders, 5th Edition (American Psychiatric Association: Diagnostic and Statistical Manual of Mental Disorders: Diagnostic and Statistical Manual of Mental Disorders, Fifth Edition. Arlington, VA: American Psychiatric Association, 2013), which is hereby incorporated by reference, describes opioid use disorder as a problematic pattern of opioid use leading to problems or distress, with at least two of the following occurring within a 12-month period:
Taking larger amounts or taking drugs over a longer period than intended.
Persistent desire or unsuccessful efforts to cut down or control opioid use.
Spending a great deal of time obtaining or using the opioid or recovering from its effects.
Craving, or a strong desire or urge to use opioids.
Problems fulfilling obligations at work, school, or home.
Continued opioid use despite having recurring social or interpersonal problems.
Giving up or reducing activities because of opioid use.
Using opioids in physically hazardous situations.
Continued opioid use despite ongoing physical or psychological problem likely to have been caused or worsened by opioids.
Tolerance (i.e., need for increased amounts or diminished effect with continued use of the same amount).
Experiencing withdrawal (opioid withdrawal syndrome) or taking opioids (or a closely related substance) to relieve or avoid withdrawal symptoms.

Alcohol use disorder (AUD) involves, but is not limited to, a chronic relapsing brain disease characterized by compulsive alcohol use, loss of control over alcohol intake, and a negative emotional state when not using. The Diagnostic and Statistical Manual of Mental Disorders, 5th Edition describes alcohol use disorder as a problematic pattern of alcohol use leading to problems or distress, with at least two of the following occurring within a 12-month period:
Being unable to limit the amount of alcohol you drink.
Wanting to cut down on how much you drink or making unsuccessful attempts to do so.
Spending a lot of time drinking, getting alcohol, or recovering from alcohol use.
Feeling a strong craving or urge to drink alcohol.
Failing to fulfill major obligations at work, school or home due to repeated alcohol use.
Continuing to drink alcohol even though you know it is causing physical, social, or interpersonal problems.
Giving up or reducing social and work activities and hobbies.
Using alcohol in situations where it is not safe, such as when driving or swimming.
Developing a tolerance to alcohol so you need more to feel its effect, or you have a reduced effect from the same amount.
Experiencing withdrawal symptoms—such as nausea, sweating and shaking—when you do not drink, or drinking to avoid these symptoms.

Stimulant use disorder involves, but is not limited to, a pattern of problematic use of amphetamine, methamphetamine, cocaine, or other stimulants except caffeine or nicotine, leading to at least two of the following problems within a 12-month period:
Taking more stimulants than intended.
Unsuccessful in trying to cut down or control use of stimulants, despite wanting to do so.
Spending excessive amounts of time to activities surrounding stimulant use.
Urges and cravings for stimulants.
Failing in the obligations of home, school, or work.
Carrying on taking stimulants, even though it has led to relationship or social problems.
Giving up or reducing important recreational, social, or work-related activities because of using stimulants.
Using stimulants in a physically hazardous way.
Continuing to use stimulants even while knowing that it is causing or worsening a physical or psychological problem.
Tolerance to stimulants.
Withdrawal from stimulants if you do not take them.

Polydrug use disorder or polysubstance use disorder involves, but is not limited to, dependence on multiple drugs or substances.

The term “MOR agonist” is intended to mean any compound or substance that activates the mu-opioid receptor (MOR). The agonist may be a partial, full, super, or biased agonist.

The term “DOR agonist” is intended to mean any compound or substance that activates the delta-opioid receptor (DOR). The agonist may be a partial, full, super, or biased agonist.

The term “KOR agonist” is intended to mean any compound or substance that activates the kappa-opioid receptor (KOR). The agonist may be a partial, full, super, or biased agonist.

The term “MOR antagonist” is intended to mean any compound or substance that blocks or inhibits the mu-opioid receptor (MOR). The antagonist may be a competitive, non-competitive, uncompetitive or silent antagonist.

The term “DOR antagonist” is intended to mean any compound or substance that blocks or inhibits the delta-opioid receptor (DOR). The antagonist may be a competitive, non-competitive, uncompetitive or silent antagonist.

The term “KOR antagonist” is intended to mean any compound or substance that blocks or inhibits the kappa-opioid receptor (KOR). The antagonist may be a competitive, non-competitive, uncompetitive or silent antagonist.

Except where otherwise specified, the structure of a compound of this invention includes an asymmetric carbon atom, it is understood that the compound occurs as a racemate, racemic mixture, and isolated single enantiomer. All such isomeric forms of these compounds are expressly included in this invention. Except where otherwise specified, each stereogenic carbon may be of the R or S configuration. It is to be understood accordingly that the isomers arising from such asymmetry (e.g., all enantiomers and diastereomers) are included within the scope of this invention, unless indicated otherwise. Such isomers can be obtained in substantially pure form by classical separation techniques and by stereochemically controlled synthesis, such as those described in “Enantiomers, Racemates and Resolutions” by J. Jacques, A. Collet and S. Wilen, Pub. John Wiley & Sons, NY, 1981. For example, the resolution may be carried out by preparative chromatography on a chiral column.

The subject invention is also intended to include all isotopes of atoms occurring on the compounds disclosed herein. Isotopes include those atoms having the same atomic number but different mass numbers. By way of general example and without limitation, isotopes of hydrogen include tritium and deuterium. Isotopes of carbon include C-13 and C-14.

It will be noted that any notation of a carbon in structures throughout this application, when used without further notation, are intended to represent all isotopes of carbon, such as ¹²C, ¹³C, or ¹⁴C. Furthermore, any compounds containing ¹³C or ¹⁴C may specifically have the structure of any of the compounds disclosed herein.

It will also be noted that any notation of a hydrogen in structures throughout this application, when used without further notation, are intended to represent all isotopes of hydrogen, such as ¹H, ²H, or ³H.

Furthermore, any compounds containing ²H or ³H may specifically have the structure of any of the compounds disclosed herein.

Isotopically-labeled compounds can generally be prepared by conventional techniques known to those skilled in the art using appropriate isotopically-labeled reagents in place of the non-labeled reagents employed.

In the compounds used in the method of the present invention, the substituents may be substituted or unsubstituted, unless specifically defined otherwise.

In the compounds used in the method of the present invention, alkyl, heteroalkyl, monocycle, bicycle, aryl, heteroaryl and heterocycle groups can be further substituted by replacing one or more hydrogen atoms with alternative non-hydrogen groups. These include, but are not limited to, halo, hydroxy, mercapto, amino, carboxy, cyano and carbamoyl.

It is understood that substituents and substitution patterns on the compounds used in the method of the present invention can be selected by one of ordinary skill in the art to provide compounds that are chemically stable and that can be readily synthesized by techniques known in the art from readily available starting materials. If a substituent is itself substituted with more than one group, it is understood that these multiple groups may be on the same carbon or on different carbons, so long as a stable structure results.

In choosing the compounds used in the method of the present invention, one of ordinary skill in the art will recognize that the various substituents, i.e. R₁, R₂, etc. are to be chosen in conformity with well-known principles of chemical structure connectivity.

As used herein, “alkyl” is intended to include both branched and straight-chain saturated aliphatic hydrocarbon groups having the specified number of carbon atoms. Thus, C₁-Cₙ as in “C₁-Cₙ alkyl” is defined to include groups having 1, 2 . . . , n−1 or n carbons in a linear or branched arrangement, and specifically includes methyl, ethyl, propyl, butyl, pentyl, hexyl, heptyl, isopropyl, isobutyl, sec-butyl and so on. An embodiment can be C₁-C₁₂ alkyl, C₂-C₁₂ alkyl, C₃-C₁₂ alkyl, C₄-C₁₂ alkyl and so on. An embodiment can be C₁-C₈ alkyl, C₂-C₈ alkyl, C₃-C₈ alkyl, C₄-C₈ alkyl and so on. “Alkoxy” represents an alkyl group as described above attached through an oxygen bridge.

The term “alkenyl” refers to a non-aromatic hydrocarbon radical, straight or branched, containing at least 1 carbon to carbon-to-carbon double bond, and up to the maximum possible number of non-aromatic carbon-carbon double bonds may be present. Thus, C₂-Cₙ alkenyl is defined to include groups having 1, 2 . . . , n−1 or n carbons. For example, “C₂-C₆ alkenyl” means an alkenyl radical having 2, 3, 4, 5, or 6 carbon atoms, and at least 1 carbon-carbon double bond, and up to, for example, 3 carbon-carbon double bonds in the case of a C₆ alkenyl, respectively. Alkenyl groups include ethenyl, propenyl, butenyl and cyclohexenyl. As described above with respect to alkyl, the straight, branched or cyclic portion of the alkenyl group may contain double bonds and may be substituted if a substituted alkenyl group is indicated. An embodiment can be C₂-C₁₂ alkenyl or C₂-Cₑ alkenyl.

The term “alkynyl” refers to a hydrocarbon radical straight or branched, containing at least 1 carbon-to-carbon triple bond, and up to the maximum possible number of non-aromatic carbon-carbon triple bonds may be present. Thus, C₂-Cₙ alkynyl is defined to include groups having 1, 2 . . . , n−1 or n carbons. For example, “C₂-C₆ alkynyl” means an alkynyl radical having 2 or 3 carbon atoms, and 1 carbon-carbon triple bond, or having 4 or 5 carbon atoms, and up to 2 carbon-carbon triple bonds, or having 6 carbon atoms, and up to 3 carbon-carbon triple bonds. Alkynyl groups include ethynyl, propynyl and butynyl.

As described above with respect to alkyl, the straight or branched portion of the alkynyl group may contain triple bonds and may be substituted if a substituted alkynyl group is indicated. An embodiment can be a C₂-Cₙ alkynyl. An embodiment can be C₂-C₁₂ alkynyl or C₃-Cₑ alkynyl.

As used herein, “hydroxyalkyl” includes alkyl groups as described above wherein one or more bonds to hydrogen contained therein are replaced by a bond to an —OH group. In some embodiments, C₁-C₁₂ hydroxyalkyl or C₁-C₆ hydroxyalkyl. C₁-Cₙ as in “C₁-Cₙ alkyl” is defined to include groups having 1, 2, . . . , n−1 or n carbons in a linear or branched arrangement (e.g. C₁-C₂ hydroxyalkyl, C₁-C₃ hydroxyalkyl, C₁-C₄ hydroxyalkyl, C₁-C₅ hydroxyalkyl, or C₁-C₆ hydroxyalkyl) For example, C₁-C₆, as in “C₁-C₆ hydroxyalkyl” is defined to include groups having 1, 2, 3, 4, 5, or 6 carbons in a linear or branched alkyl arrangement wherein a hydrogen contained therein is replaced by a bond to an —OH group.

As used herein, “heteroalkyl” includes both branched and straight-chain saturated aliphatic hydrocarbon groups having the specified number of carbon atoms and at least 1 heteroatom within the chain or branch.

In some embodiments, the haloalkyl is fluoroalkyl. In some embodiments, the fluoroalkyl is —CF₃ or —CH₂F.

As used herein, “monocycle” includes any stable polyatomic carbon ring of up to 10 atoms and may be unsubstituted or substituted. Examples of such non-aromatic monocycle elements include but are not limited to: cyclobutyl, cyclopentyl, cyclohexyl, and cycloheptyl. Examples of such aromatic monocycle elements include but are not limited to: phenyl.

As used herein, “bicycle” includes any stable polyatomic carbon ring of up to 10 atoms that is fused to a polyatomic carbon ring of up to 10 atoms with each ring being independently unsubstituted or substituted. Examples of such non-aromatic bicycle elements include but are not limited to: decahydronaphthalene. Examples of such aromatic bicycle elements include but are not limited to: naphthalene.

As used herein, “aryl” is intended to mean any stable monocyclic, bicyclic or polycyclic carbon ring of up to 10 atoms in each ring, wherein at least one ring is aromatic, and may be unsubstituted or substituted. Examples of such aryl elements include but are not limited to: phenyl, p-toluenyl (4-methylphenyl), naphthyl, tetrahydro-naphthyl, indanyl, phenanthryl, anthryl or acenaphthyl. In cases where the aryl substituent is bicyclic and one ring is non-aromatic, it is understood that attachment is via the aromatic ring.

The term “heteroaryl”, as used herein, represents a stable monocyclic, bicyclic or polycyclic ring of up to 10 atoms in each ring, wherein at least one ring is aromatic and contains from 1 to 4 heteroatoms selected from the group consisting of O, N and S. Bicyclic aromatic heteroaryl groups include phenyl, pyridine, pyrimidine or pyridazine rings that are (a) fused to a 6-membered aromatic (unsaturated) heterocyclic ring having one nitrogen atom; (b) fused to a 5- or 6-membered aromatic (unsaturated) heterocyclic ring having two nitrogen atoms; (c) fused to a 5-membered aromatic (unsaturated) heterocyclic ring having one nitrogen atom together with either one oxygen or one sulfur atom; or (d) fused to a 5-membered aromatic (unsaturated) heterocyclic ring having one heteroatom selected from O, N or S. Heteroaryl groups within the scope of this definition include but are not limited to: benzoimidazolyl, benzofuranyl, benzofurazanyl, benzopyrazolyl, benzotriazolyl, benzothiophenyl, benzoxazolyl, carbazolyl, carbolinyl, cinnolinyl, furanyl, indolinyl, indolyl, indolazinyl, indazolyl, isobenzofuranyl, isoindolyl, isoquinolyl, isothiazolyl, isoxazolyl, naphthpyridinyl, oxadiazolyl, oxazolyl, oxazoline, isoxazoline, oxetanyl, pyranyl, pyrazinyl, pyrazolyl, pyridazinyl, pyridopyridinyl, pyridazinyl, pyridyl, pyrimidyl, pyrrolyl, quinazolinyl, quinolyl, quinoxalinyl, tetrazolyl, tetrazolopyridyl, thiadiazolyl, thiazolyl, thienyl, triazolyl, azetidinyl, aziridinyl, 1,4-dioxanyl, hexahydroazepinyl, dihydrobenzoimidazolyl, dihydrobenzofuranyl, dihydrobenzothiophenyl, dihydrobenzoxazolyl, dihydrofuranyl, dihydroimidazolyl, dihydroindolyl, dihydroisooxazolyl, dihydroisothiazolyl, dihydrooxadiazolyl, dihydrooxazolyl, dihydropyrazinyl, dihydropyrazolyl, dihydropyridinyl, dihydropyrimidinyl, dihydropyrrolyl, dihydroquinolinyl, dihydrotetrazolyl, dihydrothiadiazolyl, dihydrothiazolyl, dihydrothienyl, dihydrotriazolyl, dihydroazetidinyl, methylenedioxybenzoyl, tetrahydrofuranyl, tetrahydrothienyl, acridinyl, carbazolyl, cinnolinyl, quinoxalinyl, pyrrazolyl, indolyl, benzotriazolyl, benzothiazolyl, benzoxazolyl, isoxazolyl, isothiazolyl, furanyl, thienyl, benzothienyl, benzofuranyl, quinolinyl, isoquinolinyl, oxazolyl, isoxazolyl, indolyl, pyrazinyl, pyridazinyl, pyridinyl, pyrimidinyl, pyrrolyl, tetra-hydroquinoline. In cases where the heteroaryl substituent is bicyclic and one ring is non-aromatic or contains no heteroatoms, it is understood that attachment is via the aromatic ring or via the heteroatom containing ring, respectively. If the heteroaryl contains nitrogen atoms, it is understood that the corresponding N-oxides thereof are also encompassed by this definition.

The term “heterocycle”, “heterocyclyl” or “heterocyclic” refers to a mono- or poly-cyclic ring system which can be saturated or contains one or more degrees of unsaturation and contains one or more heteroatoms. Preferred heteroatoms include N, O, and/or S, including N-oxides, sulfur oxides, and dioxides. Preferably the ring is three to ten-membered and is either saturated or has one or more degrees of unsaturation. The heterocycle may be unsubstituted or substituted, with multiple degrees of substitution being allowed. Such rings may be optionally fused to one or more of another “heterocyclic” ring(s), heteroaryl ring(s), aryl ring(s), or cycloalkyl ring(s). Examples of heterocycles include, but are not limited to, tetrahydrofuran, pyran, 1,4-dioxane, 1,3-dioxane, piperidine, piperazine, pyrrolidine, morpholine, thiomorpholine, tetrahydrothiopyran, tetrahydrothiophene, 1,3-oxathiolane, and the like.

As used herein, “cycloalkyl” includes cyclic rings of alkanes of three to eight total carbon atoms, or any number within this range (i.e., cyclopropyl, cyclobutyl, cyclopentyl, cyclohexyl, cycloheptyl or cyclooctyl).

The term “ester” is intended to a mean an organic compound containing the R—O—CO—R′ group.

The term “amide” is intended to a mean an organic compound containing the R—CO—NH—R′ or R—CO—N—R′R″ group.

The term “phenyl” is intended to mean an aromatic six membered ring containing six carbons.

The term “benzyl” is intended to mean a —CH₂R₁ group wherein the R₁ is a phenyl group.

The term “substitution”, “substituted” and “substituent” refers to a functional group as described above in which one or more bonds to a hydrogen atom contained therein are replaced by a bond to non-hydrogen or non-carbon atoms, provided that normal valencies are maintained and that the substitution results in a stable compound. Substituted groups also include groups in which one or more bonds to a carbon(s) or hydrogen(s) atom are replaced by one or more bonds, including double or triple bonds, to a heteroatom. Examples of substituent groups include the functional groups described above, and halogens (i.e., F, Cl, Br, and I); alkyl groups, such as methyl, ethyl, n-propyl, isopropyl, n-butyl, tert-butyl, and trifluoromethyl; hydroxyl; alkoxy groups, such as methoxy, ethoxy, n-propoxy, and isopropoxy; aryloxy groups, such as phenoxy; arylalkyloxy, such as benzyloxy (phenylmethoxy) and p-trifluoromethylbenzyloxy (4-trifluoromethylphenylmethoxy); heteroaryloxy groups; sulfonyl groups, such as trifluoromethanesulfonyl, methanesulfonyl, and p-toluenesulfonyl; nitro, nitrosyl; mercapto; sulfanyl groups, such as methylsulfanyl, ethylsulfanyl and propylsulfanyl; cyano; amino groups, such as amino, methylamino, dimethylamino, ethylamino, and diethylamino; and carboxyl. Where multiple substituent moieties are disclosed or claimed, the substituted compound can be independently substituted by one or more of the disclosed or claimed substituent moieties, singly or plurally. By independently substituted, it is meant that the (two or more) substituents can be the same or different.

The compounds used in the method of the present invention may be prepared by techniques well known in organic synthesis and familiar to a practitioner ordinarily skilled in the art. However, these may not be the only means by which to synthesize or obtain the desired compounds.

The compounds used in the method of the present invention may be prepared by techniques described in Vogel's Textbook of Practical Organic Chemistry, A. I. Vogel, A. R. Tatchell, B. S. Furnis, A. J. Hannaford, P. W. G. Smith, (Prentice Hall) 5^{th} Edition (1996), March's Advanced Organic Chemistry: Reactions, Mechanisms, and Structure, Michael B. Smith, Jerry March, (Wiley-Interscience) 5^{th} Edition (2007), and references therein, which are incorporated by reference herein. However, these may not be the only means by which to synthesize or obtain the desired compounds.

The various R groups attached to the aromatic rings of the compounds disclosed herein may be added to the rings by standard procedures, for example those set forth in Advanced Organic Chemistry: Part B: Reactions and Synthesis, Francis Carey and Richard Sundberg, (Springer) 5th ed. Edition. (2007), the content of which is hereby incorporated by reference.

Another aspect of the invention comprises a compound used in the method of the present invention as a pharmaceutical composition.

As used herein, the term “pharmaceutically active agent” means any substance or compound suitable for administration to a subject and furnishes biological activity or other direct effect in the treatment, cure, mitigation, diagnosis, or prevention of disease, or affects the structure or any function of the subject. Pharmaceutically active agents include, but are not limited to, substances and compounds described in the Physicians' Desk Reference (PDR Network, LLC; 64th edition; Nov. 15, 2009) and “Approved Drug Products with Therapeutic Equivalence Evaluations” (U.S. Department Of Health And Human Services, 30^{th} edition, 2010), which are hereby incorporated by reference. Pharmaceutically active agents which have pendant carboxylic acid groups may be modified in accordance with the present invention using standard esterification reactions and methods readily available and known to those having ordinary skill in the art of chemical synthesis. Where a pharmaceutically active agent does not possess a carboxylic acid group, the ordinarily skilled artisan will be able to design and incorporate a carboxylic acid group into the pharmaceutically active agent where esterification may subsequently be carried out so long as the modification does not interfere with the pharmaceutically active agent's biological activity or effect.

The compounds used in the method of the present invention may be in a salt form. As used herein, a “salt” is a salt of the instant compounds which has been modified by making acid or base salts of the compounds. In the case of compounds used to treat an infection or disease caused by a pathogen, the salt is pharmaceutically acceptable. Examples of pharmaceutically acceptable salts include, but are not limited to, mineral or organic acid salts of basic residues such as amines; alkali or organic salts of acidic residues such as phenols. The salts can be made using an organic or inorganic acid. Such acid salts are chlorides, bromides, sulfates, nitrates, phosphates, sulfonates, formates, tartrates, maleates, malates, citrates, benzoates, salicylates, ascorbates, and the like. Phenolate salts are the alkaline earth metal salts, sodium, potassium or lithium. The term “pharmaceutically acceptable salt” in this respect, refers to the relatively non-toxic, inorganic and organic acid or base addition salts of compounds of the present invention. These salts can be prepared in situ during the final isolation and purification of the compounds of the invention, or by separately reacting a purified compound of the invention in its free base or free acid form with a suitable organic or inorganic acid or base, and isolating the salt thus formed. Representative salts include the hydrobromide, hydrochloride, sulfate, bisulfate, phosphate, nitrate, acetate, valerate, oleate, palmitate, stearate, laurate, benzoate, lactate, phosphate, tosylate, citrate, maleate, fumarate, succinate, tartrate, napthylate, mesylate, glucoheptonate, lactobionate, and laurylsulphonate salts and the like. (See, e.g., Berge et al. (1977) “Pharmaceutical Salts”, J. Pharm. Sci. 66:1-19).

As used herein, “treating” means preventing, slowing, halting, or reversing the progression of a disease or infection. Treating may also mean improving one or more symptoms of a disease or infection.

The compounds used in the method of the present invention may be administered in various forms, including those detailed herein. The treatment with the compound may be a component of a combination therapy or an adjunct therapy, i.e. the subject or patient in need of the drug is treated or given another drug for the disease in conjunction with one or more of the instant compounds. This combination therapy can be sequential therapy where the patient is treated first with one drug and then the other or the two drugs are given simultaneously. These can be administered independently by the same route or by two or more different routes of administration depending on the dosage forms employed.

As used herein, a “pharmaceutically acceptable carrier” is a pharmaceutically acceptable solvent, suspending agent or vehicle, for delivering the instant compounds to the animal or human. The carrier may be liquid or solid and is selected with the planned manner of administration in mind. Liposomes are also a pharmaceutically acceptable carrier.

The dosage of the compounds administered in treatment will vary depending upon factors such as the pharmacodynamic characteristics of a specific chemotherapeutic agent and its mode and route of administration; the age, sex, metabolic rate, absorptive efficiency, health and weight of the recipient; the nature and extent of the symptoms; the kind of concurrent treatment being administered; the frequency of treatment with; and the desired therapeutic effect.

The compounds can be administered in oral dosage forms as tablets, capsules, pills, powders, granules, elixirs, tinctures, suspensions, syrups, and emulsions. The compounds may also be administered in intravenous (bolus or infusion), intraperitoneal, subcutaneous, or intramuscular form, or introduced directly, e.g. by injection, topical application, or other methods, into or onto a site of infection, all using dosage forms well known to those of ordinary skill in the pharmaceutical arts.

The compounds used in the method of the present invention can be administered in admixture with suitable pharmaceutical diluents, extenders, excipients, or carriers (collectively referred to herein as a pharmaceutically acceptable carrier) suitably selected with respect to the intended form of administration and as consistent with conventional pharmaceutical practices. The unit will be in a form suitable for oral, rectal, topical, intravenous or direct injection or parenteral administration. The compounds can be administered alone or mixed with a pharmaceutically acceptable carrier. This carrier can be a solid or liquid, and the type of carrier is generally chosen based on the type of administration being used. The active agent can be co-administered in the form of a tablet or capsule, liposome, as an agglomerated powder or in a liquid form. Examples of suitable solid carriers include lactose, sucrose, gelatin and agar. Capsule or tablets can be easily formulated and can be made easy to swallow or chew; other solid forms include granules, and bulk powders. Tablets may contain suitable binders, lubricants, diluents, disintegrating agents, coloring agents, flavoring agents, flow-inducing agents, and melting agents. Examples of suitable liquid dosage forms include solutions or suspensions in water, pharmaceutically acceptable fats and oils, alcohols or other organic solvents, including esters, emulsions, syrups or elixirs, suspensions, solutions and/or suspensions reconstituted from non-effervescent granules and effervescent preparations reconstituted from effervescent granules.

Such liquid dosage forms may contain, for example, suitable solvents, preservatives, emulsifying agents, suspending agents, diluents, sweeteners, thickeners, and melting agents. Oral dosage forms optionally contain flavorants and coloring agents. Parenteral and intravenous forms may also include minerals and other materials to make them compatible with the type of injection or delivery system chosen.

Techniques and compositions for making dosage forms useful in the present invention are described in the following references: 7 Modern Pharmaceutics, Chapters 9 and 10 (Banker & Rhodes, Editors, 1979); Pharmaceutical Dosage Forms: Tablets (Lieberman et al., 1981); Ansel, Introduction to Pharmaceutical Dosage Forms 2nd Edition (1976); Remington's Pharmaceutical Sciences, 17th ed. (Mack Publishing Company, Easton, Pa., 1985); Advances in Pharmaceutical Sciences (David Ganderton, Trevor Jones, Eds., 1992); Advances in Pharmaceutical Sciences Vol. 7. (David Ganderton, Trevor Jones, James McGinity, Eds., 1995); Aqueous Polymeric Coatings for Pharmaceutical Dosage Forms (Drugs and the Pharmaceutical Sciences, Series 36 (James McGinity, Ed., 1989); Pharmaceutical Particulate Carriers: Therapeutic Applications: Drugs and the Pharmaceutical Sciences, Vol 61 (Alain Rolland, Ed., 1993); Drug Delivery to the Gastrointestinal Tract (Ellis Horwood Books in the Biological Sciences. Series in Pharmaceutical Technology; J. G. Hardy, S. S. Davis, Clive G. Wilson, Eds.); Modem Pharmaceutics Drugs and the Pharmaceutical Sciences, Vol 40 (Gilbert S. Banker, Christopher T. Rhodes, Eds.). All of the aforementioned publications are incorporated by reference herein.

Tablets may contain suitable binders, lubricants, disintegrating agents, coloring agents, flavoring agents, flow-inducing agents, and melting agents. For instance, for oral administration in the dosage unit form of a tablet or capsule, the active drug component can be combined with an oral, non-toxic, pharmaceutically acceptable, inert carrier such as lactose, gelatin, agar, starch, sucrose, glucose, methyl cellulose, magnesium stearate, dicalcium phosphate, calcium sulfate, mannitol, sorbitol and the like. Suitable binders include starch, gelatin, natural sugars such as glucose or beta-lactose, corn sweeteners, natural and synthetic gums such as acacia, tragacanth, or sodium alginate, carboxymethylcellulose, polyethylene glycol, waxes, and the like. Lubricants used in these dosage forms include sodium oleate, sodium stearate, magnesium stearate, sodium benzoate, sodium acetate, sodium chloride, and the like. Disintegrators include, without limitation, starch, methyl cellulose, agar, bentonite, xanthan gum, and the like.

The compounds used in the method of the present invention may also be administered in the form of liposome delivery systems, such as small unilamellar vesicles, large unilamellar vesicles, and multilamellar vesicles. Liposomes can be formed from a variety of phospholipids, such as cholesterol, stearylamine, or phosphatidylcholines. The compounds may be administered as components of tissue-targeted emulsions.

The compounds used in the method of the present invention may also be coupled to soluble polymers as targetable drug carriers or as a prodrug. Such polymers include polyvinylpyrrolidone, pyran copolymer, polyhydroxylpropylmethacrylamide-phenol, polyhydroxyethylasparta-midephenol, or polyethyleneoxide-polylysine substituted with palmitoyl residues. Furthermore, the compounds may be coupled to a class of biodegradable polymers useful in achieving controlled release of a drug, for example, polylactic acid, polyglycolic acid, copolymers of polylactic and polyglycolic acid, polyepsilon caprolactone, polyhydroxy butyric acid, polyorthoesters, polyacetals, polydihydropyrans, polycyanoacylates, and crosslinked or amphipathic block copolymers of hydrogels.

Gelatin capsules may contain the active ingredient compounds and powdered carriers, such as lactose, starch, cellulose derivatives, magnesium stearate, stearic acid, and the like. Similar diluents can be used to make compressed tablets. Both tablets and capsules can be manufactured as immediate release products or as sustained release products to provide for continuous release of medication over a period of hours. Compressed tablets can be sugar coated or film coated to mask any unpleasant taste and protect the tablet from the atmosphere, or enteric coated for selective disintegration in the gastrointestinal tract.

For oral administration in liquid dosage form, the oral drug components are combined with any oral, non-toxic, pharmaceutically acceptable inert carrier such as ethanol, glycerol, water, and the like. Examples of suitable liquid dosage forms include solutions or suspensions in water, pharmaceutically acceptable fats and oils, alcohols or other organic solvents, including esters, emulsions, syrups or elixirs, suspensions, solutions and/or suspensions reconstituted from non-effervescent granules and effervescent preparations reconstituted from effervescent granules. Such liquid dosage forms may contain, for example, suitable solvents, preservatives, emulsifying agents, suspending agents, diluents, sweeteners, thickeners, and melting agents.

Liquid dosage forms for oral administration can contain coloring and flavoring to increase patient acceptance. In general, water, a suitable oil, saline, aqueous dextrose (glucose), and related sugar solutions and glycols such as propylene glycol or polyethylene glycols are suitable carriers for parenteral solutions. Solutions for parenteral administration preferably contain a water soluble salt of the active ingredient, suitable stabilizing agents, and if necessary, buffer substances. Antioxidizing agents such as sodium bisulfite, sodium sulfite, or ascorbic acid, either alone or combined, are suitable stabilizing agents. Also used are citric acid and its salts and sodium EDTA. In addition, parenteral solutions can contain preservatives, such as benzalkonium chloride, methyl- or propyl-paraben, and chlorobutanol. Suitable pharmaceutical carriers are described in Remington's Pharmaceutical Sciences, Mack Publishing Company, a standard reference text in this field.

The compounds used in the method of the present invention may also be administered in intranasal form via use of suitable intranasal vehicles, or via transdermal routes, using those forms of transdermal skin patches well known to those of ordinary skill in that art. To be administered in the form of a transdermal delivery system, the dosage administration will generally be continuous rather than intermittent throughout the dosage regimen.

Parenteral and intravenous forms may also include minerals and other materials to make them compatible with the type of injection or delivery system chosen.

Each embodiment disclosed herein is contemplated as being applicable to each of the other disclosed embodiments. Thus, all combinations of the various elements described herein are within the scope of the invention.

This invention will be better understood by reference to the Experimental Details which follow, but those skilled in the art will readily appreciate that the specific experiments detailed are only illustrative of the invention as described more fully in the claims which follow thereafter.

### EXPERIMENTAL DETAILS

### Example 1. Pro-Arrhythmia Assay Analysis of Noribogaine, Oxa-Noribogaine, Epi-Oxa-Noribogaine and Desethyl-Oxa-Noribogaine Analogs in Adult Human Primary Ventricular Cardiomyocytes

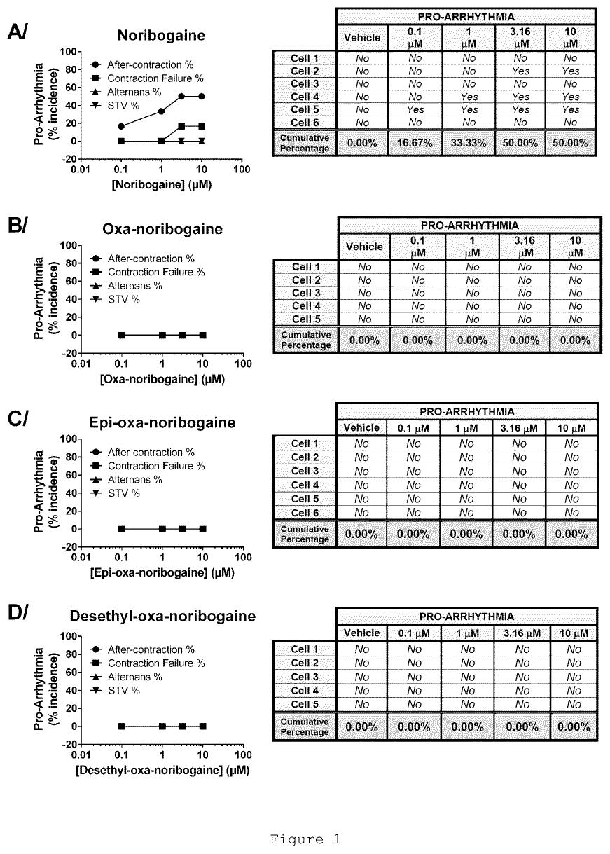

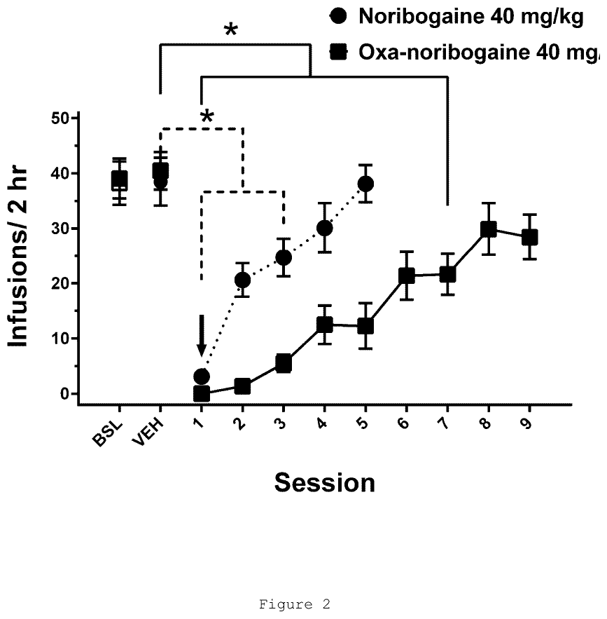

Cardiac adverse effects of iboga alkaloids and related analogs was assessed via a commercially available assay, procedure reported in (Nguyen, N. et al. 2017; Page, G. et al. 2016) (FIG. 1).

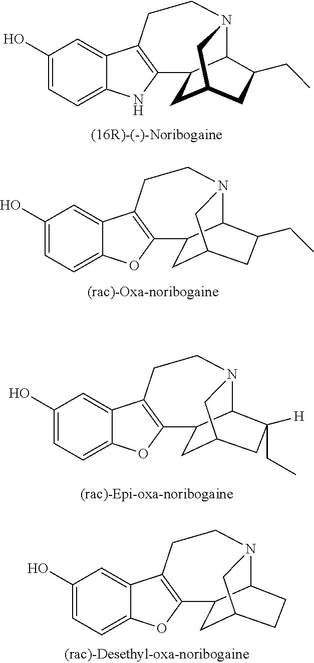

Isolation of adult human primary ventricular myocytes. Adult human primary ventricular myocytes were isolated from ethically consented donor hearts. A proprietary protocol was employed to enzymatically digest hearts and isolate cardiomyocytes.

Contractility recordings procedures. Cardiomyocytes were placed in a perfusion chamber mounted on the stage of inverted Motic AE31E (IonOptix) or Olympus IX83P1ZF microscopes (MyoBlazer) and continuously perfused at approximately 2 ml/min with recording buffer heated to 35±1° C. using an in-line heater from Warner Instruments (IonOptix & MyoBlazer) and allowed to equilibrate for 5 minutes under constant perfusion. The cells were field stimulated with supra-threshold voltage at a 1 Hz pacing frequency, with a bipolar pulse of 3 ms duration, using a pair of platinum wires placed on opposite sides of the chamber connected to a MyoPacer stimulator. Starting at 1 V, the amplitude of the stimulating pulse was increased until the cardiomyocytes started generating contraction-relaxation cycles, and a value 1.5× threshold was used throughout the experiment. Cardiomyocytes were then imaged at 240 Hz using an IonOptix MyoCam-S CCD camera (IonOptix) or at 148 Hz using an Optronis CP70-16-M/C-148 (MyoBLAZER) camera. Digitized images were displayed within the IonWizard acquisition software (IonOptix) or MyoBLAZER acquisition software. The longitudinal axis of the selected cardiomyocyte was aligned parallel to the video raster line, by means of a cell framing adapter. Optical intensity data was collected from a user-defined rectangular region placed over the cardiomyocyte image. The optical intensity data represented the bright and dark bands corresponding to the Z-lines of the cardiomyocyte. The IonWizard software or MyoBLAZER Analysis software analyzed the periodicity in the optical density of these bands by means of a fast Fourier transform algorithm.

Pro-arrhythmia markers. Aftercontractions (AC) were identified as spontaneous secondary contraction transients of the cardiomyocyte that occurred before the next regular contraction and that produced an abnormal and unsynchronized contraction. Contraction Failure (CF) was identified when an electrical stimulus was unable to induce a contraction. Alternans and Short-Term Variability (STV) are visualized in Poincare plots of Contraction Amplitude variability. STV (STV=Σ|CAn+1−CAn|(20×√2)⁻¹) was calculated with the last 20 transients of each control and test article concentration period. Alternans were identified as repetitive alternating short and long contractility amplitude transients. STV values were normalized to the vehicle control value of each cell. AC, CF and Alternans were plotted and expressed as % of incidence of cells exhibiting each of the signals.

### Evaluation Parameters

Pro-Arrhythmia
After-Contraction (AC) (%)
Contractility Failure (CF) (%)
Alternans (%)
Short Term Variability of CA (STV(CA))

TABLE 1
Stimulation protocol and test article application sequence.

| Perfusion | Vehicle | | Test Article Concentration | | | | | | | | | |
|---|---|---|---|---|---|---|---|---|---|---|---|---|
| Sequence | Control | | 0.1 μM | | 1 μM | | 3.16 μM | | 10 μM | | Wash | |
| Treatment | 120 | sec | 300 | sec | 300 | sec | 300 | sec | 300 | sec | 300 | sec |
| Time & | 1.0 | Hz | 1.0 | Hz | 1.0 | Hz | 1.0 | Hz | 1.0 | Hz | 1.0 | Hz |

Stimulation

TABLE 2
Stimulation protocol and positive control application sequence.

| | | |
|---|---|---|
| Perfusion Sequence | Vehicle Control | 30 nM ATX-II |

| | | | | |
|---|---|---|---|---|
| Treatment Time | 120 | sec | *300 | sec |
| Stimulation | 1.0 | Hz | 1.0 | Hz |

### Example 2. Effects of Acute Administration of Oxa-Noribogaine, Epi-Oxa-Noribogaine and Noribogaine on Intravenous Drug Self-Administration in Rodent Animal Models

Subjects. Adult male Fisher F-344 rats (90-150 days; Charles River Laboratories, Wilmington, MA) were housed individually in acrylic cages with food and water available ad libitum. Rats were maintained on a 12-hr light/dark cycle with lights on at 7:00 P.M., and experimental sessions took place during the dark phase of the cycle.

Operant apparatus. Rats were transferred to operant conditioning chambers (ENV-008CT; Med Associates, St. Albans, VT) enclosed in sound-attenuating cubicles (ENV-018; Med Associates). The front panel of the operant chambers contained two response levers (4 cm above the floor and 3 cm from the side walls), a cue light (3 cm above the lever) and a food chute centered on the front wall (2 cm above the floor) that was connected to a food pellet dispenser (ENV-023; Med Associates) located behind the front wall and a tone generator to mask extraneous noise. A syringe pump (PHM-100; Med Associates) holding a 20-ml syringe delivered infusions. A counter-balanced arm containing the single channel liquid swivel was located 8-8.5 cm above the chamber and attached to the outside of the front panel. An IBM compatible computer was used for session programming and data collection (Med Associates Inc., East Fairfield, VT).

Lever training. Subjects were transferred to the operant chambers for daily experimental sessions and responding was engendered and maintained by delivery of food pellets (45 mg pellets: Noyes, Lancaster, NH) under an FR 1 schedule of reinforcement. The lever lights were illuminated when the schedule was in effect. Completion of the response requirement extinguished lights, delivered food, and was followed by a 20-second timeout (TO) period during which all lights were extinguished, and responses had no scheduled consequences. After the TO, the lights were illuminated, and the FR schedule was again in effect. Sessions lasted 20 minutes or until 40 food pellets were delivered. Responding was considered stable when there was less than 10% variation in the number of reinforcers for three consecutive sessions.

Intravenous jugular surgery. After operant responding was acquired and maintained by food, subjects surgically implanted with an intravenous jugular catheter. Venous catheters were inserted into the right jugular vein following administration of ketamine (90 mg/kg; IP) and xylazine (5 mg/kg; IP) for anesthesia (Pattison, L. P. et al., 2014; Pattison, L. P. et al., 2012; McIntosh, S. et al., 2015). Catheters were anchored to muscle near the point of entry into the vein. The distal end of the catheter was guided subcutaneously to exit above the scapulae through a Teflon shoulder harness. The harness provided a point of attachment for a spring leash connected to a single-channel fluid swivel at the opposing end. The catheter was threaded through the leash and attached to the swivel. The other end of the swivel was connected to a syringe (for saline and drug delivery) mounted on a syringe pump. Rats were administered penicillin G procaine (75,000 units in 0.25 ml, i.m.) and allowed a minimum of 5 days to recover before self-administration studies were initiated. Hourly infusions of heparinized saline (500 μl) were administered through the catheter to maintain functional catheters. The health of the rats was monitored daily by the experimenters and weekly by institutional veterinarians per the guidelines issued by the High Point University Institutional Animal Care and Use Committee and the National Institutes of Health. Infusions of propofol (6 mg/kg; i.v.) were manually administered as needed to assess catheter patency.

### Experiment 1: Effect of Oxa-Noribogaine, Epi-Oxa-Noribogaine and Noribogaine on Morphine Self-Administration

Rats were transferred to the operant chambers for daily self-administration sessions that lasted 2 hr or 50 infusions. Before each session, the swivel and catheter were flushed with 500 μl of heparinized saline before connecting the catheter to the syringe via a 20 ga luer hub and 28 ga male connector. The start of each session was indicated by the illumination of the house light, stimulus light above the lever and the extension of the lever. Completion of the response requirement was followed by a 20 sec time out (FR1:TO 20 sec) during which time the subject received a 200 μl intravenous infusion of morphine (10 μg/infusion) over the first six seconds, retraction of the levers, extinguishing of lever light, generation of a tone, and illumination of the house light. At the end of the TO, the lever was extended, lever light illuminated, tone silenced, and the house light extinguished. After a minimum of three days of stable responding (defined as total number of infusions did not vary by more than 10% from the mean of the three previous sessions), rats were administered VEH (IP; 2 ml/kg) 15 min prior to the subsequent experimental session. Three days following VEH administration, rats were administered oxa-noribogaine (10 or 40 mg/kg, i.p.), epi-oxa-noribogaine (40 mg/kg; IP) or noribogaine (40 mg/kg i.p.), administered 15 min prior to the beginning of the session. Drugs were administered in doses calculated as 0.5 mg/ml.

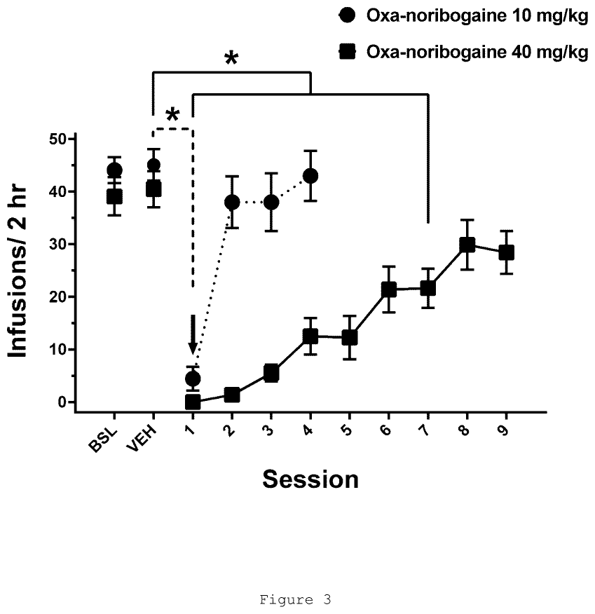

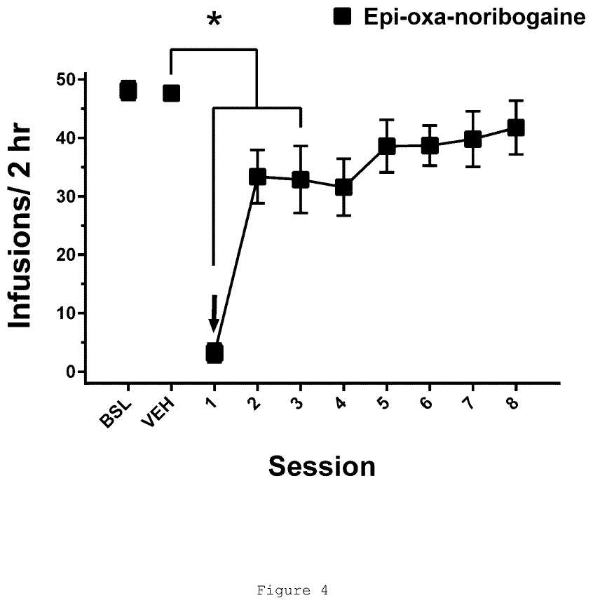

Prior to treatment, stable responding was maintained for all groups in Experiment 1. No significant difference was observed in the number of infusions obtained during baseline between the groups in Experiment 1. In addition, no significant differences in the number of infusions obtained were detected between baseline and following vehicle administration. Administration of noribogaine (40 mg/kg; IP; n=10) significantly reduced morphine intake compared to VEH for three consecutive sessions. The number of infusions on the fourth session was not significantly different than VEH. In contrast, following administration of oxa-noribogaine (40 mg/kg; IP; n=8) morphine intake remained significantly lower than baseline levels for seven consecutive sessions (FIG. 2).

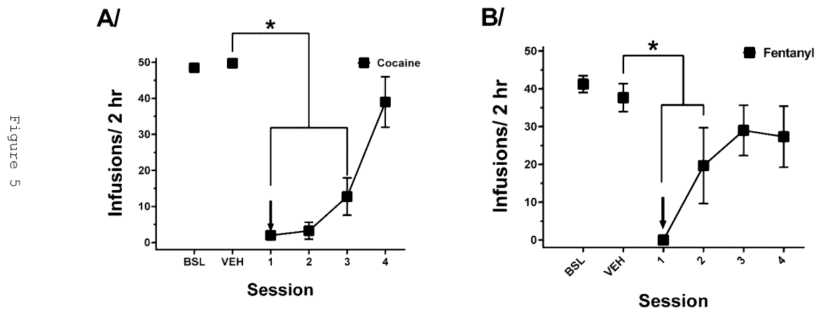

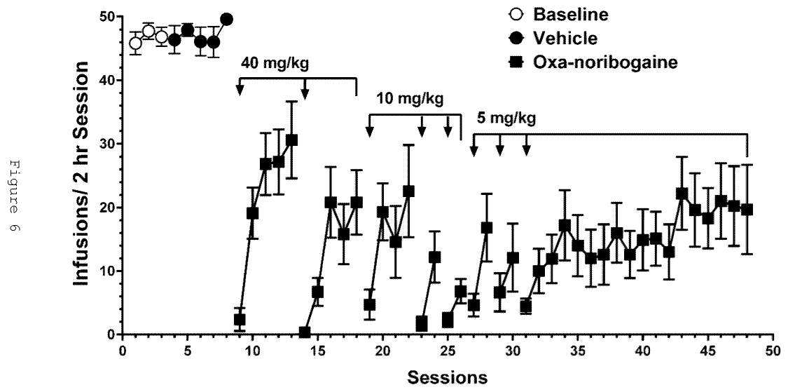

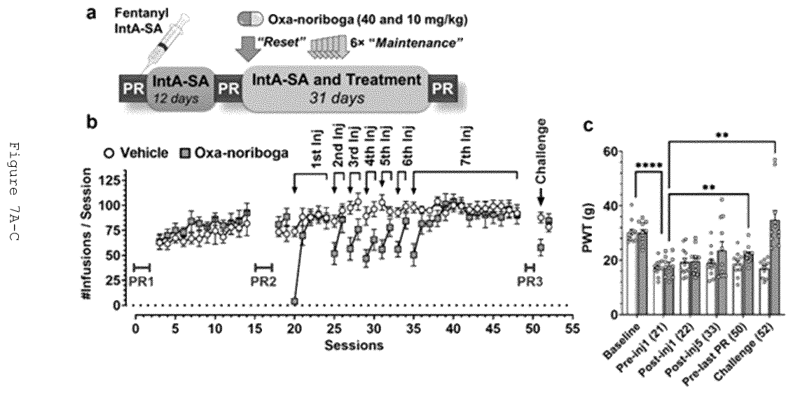

Oxa-noribogaine exerted a dose-dependent effect on morphine self-administration. For both oxa-noribogaine doses tested, the number of infusions was significantly reduced during the session immediately following administration (FIG. 3). Intake returned to baseline levels for the second session following administration of 10 mg/kg oxa-noribogaine (n=7). In contrast, morphine intake remained significantly lower than baseline levels for seven consecutive sessions following administration of 40 mg/kg oxa-noribogaine (n=8). Epi-oxa-noribogaine (40 mg/kg, IP; n=10) administration significantly decreased morphine self-administration for three sessions. Intake returned to baseline levels during the fourth session (FIG. 4).

### Experiment 2: Effect of Oxa-Noribogaine on Cocaine and Fentanyl Self-Administration

The subjects, training, surgical procedures and self-administration were identical to Experiment 1. Separate cohorts of rats were allowed to self-administer cocaine (62.5 μg/infusion; n=4) and fentanyl (625 ng/infusion; n=3) under a FR1:TO 20 sec in daily two-hour sessions or a maximum of 50 infusions. Following three days of stable responding, rats were administered VEH (IP; 2 ml/kg) 15 min prior to the subsequent experimental session. Three days following VEH administration, rats were administered oxa-noribogaine (40 mg/kg; IP) 15 min prior to the subsequent experimental session. Drug doses were administered as 0.5 mg/ml.

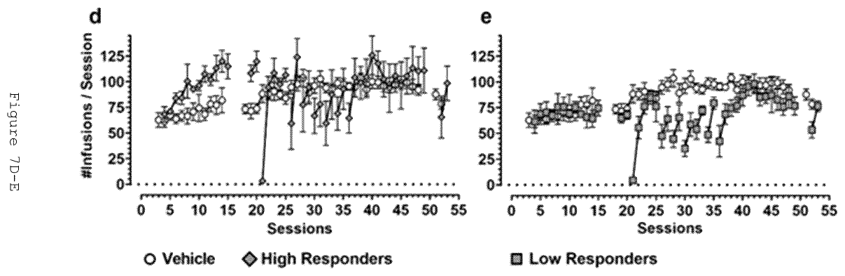

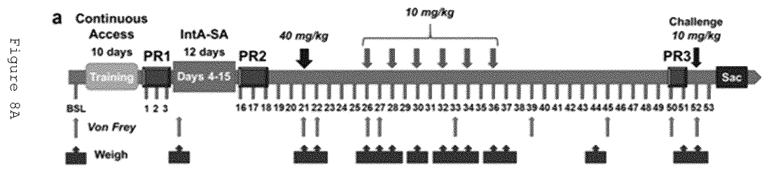

Stable responding was maintained for groups self-administering fentanyl and cocaine. No significant differences in the number of infusions obtained were detected between baseline and following vehicle administration. Acute administration of oxa-noribogaine (40 mg/kg) significantly reduced fentanyl self-administration (n=3) for two days following administration and reduced cocaine self-administration (n=4) for three days following administration (FIG. 5).

### Experiment 3: Effect of Repeated Administration of Oxa-Noribogaine on Morphine Self-Administration

The subjects, training, surgical procedures and self-administration were identical to Experiment 1. Rats were allowed to self-administer morphine (10 μg/infusion; n=11) under a FR1:TO 20 sec in daily two-hour sessions or a maximum of 50 infusions. Following three days of stable responding, rats were administered VEH (IP; 2 ml/kg) 15 min prior to the subsequent experimental session. Five days following VEH administration, a repeated administration procedure of oxa-noribogaine was initiated as follows: Days 1 and 6: 40 mg/kg, Days 11, 15 and 17: 10 mg/kg, and Days 19, 21, and 23: 5 mg/kg. The respective doses of oxa-noribogaine were administered 15 min prior to the experimental sessions as indicated above. Drug doses were administered as 0.5 mg/ml.

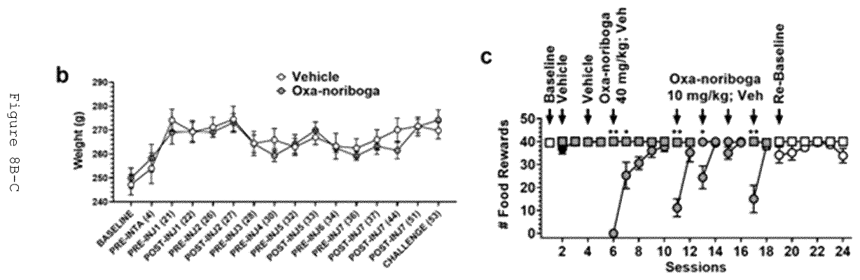

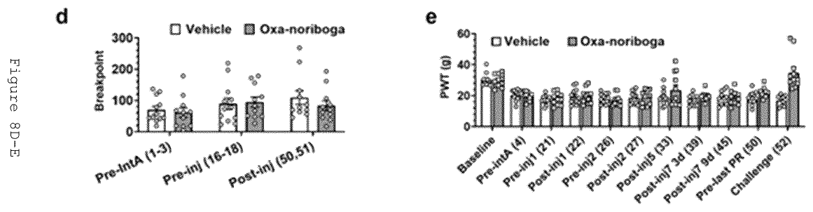

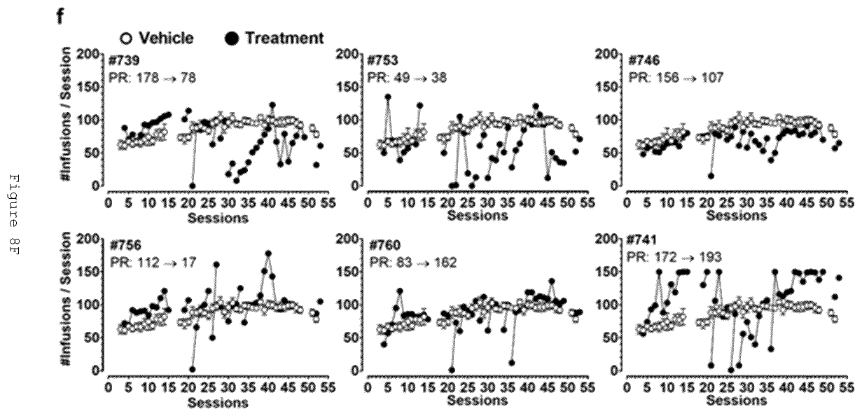

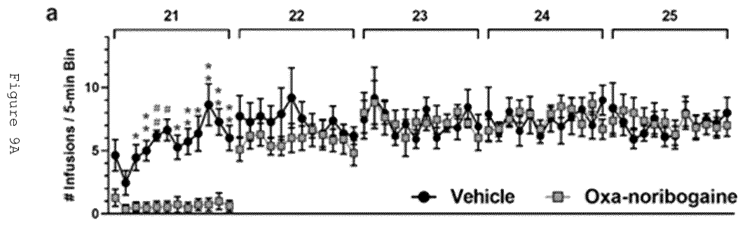

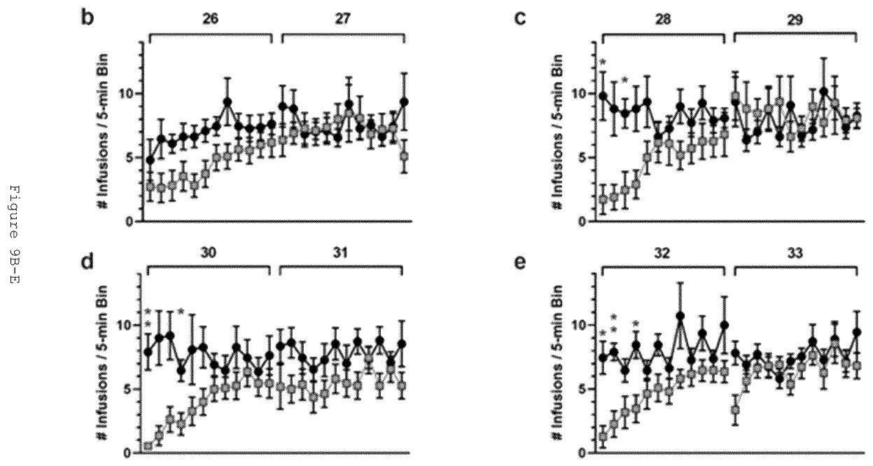

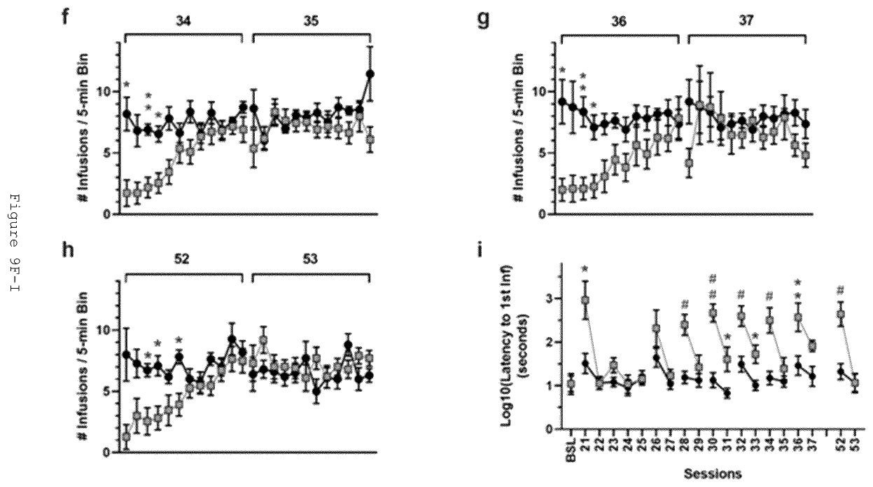

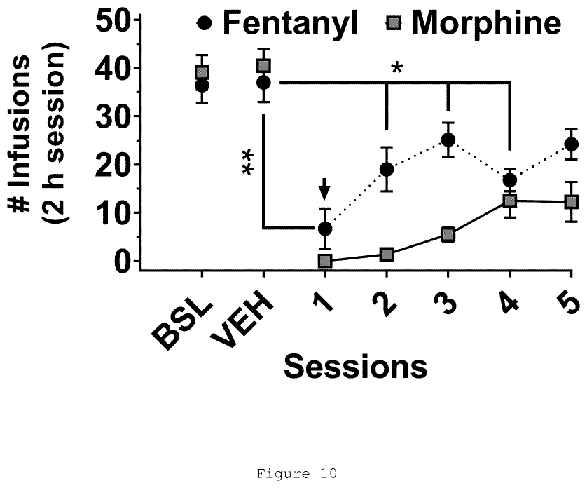

Stable responding was maintained in a separate cohort of rats self-administering morphine (10 μg/infusion). VEH administration did not significantly alter the number of infusions compared to baseline. The aforementioned oxa-noribogaine repeated dosing procedure significantly reduced morphine self-administration at all doses tested (FIG. 6).

The first administration of oxa-noribogaine 40 mg/kg essentially blocked morphine self-administration during the session immediately following oxa-noribogaine administration. Intake increased incrementally to approximately 60% of VEH levels. Morphine intake following the second administration of oxa-noribogaine 40 mg/kg was significantly reduced, but slightly higher than intake following the initial administration. Intake increased to approximately 50% over following three days. Oxa-noribogaine 10 mg/kg significantly reduced intake to approximately 4 infusions for the session following administration with intake recovering to approximately 40% of baseline levels for the following three sessions. The second administration of 10 mg/kg oxa-noribogaine blocked morphine intake during the first session and intake was increased to approximately 25% of baseline levels the following session. The third administration of 10 mg/kg oxa-noribogaine blocked morphine intake in the first session oxa-noribogaine and only slightly increased the following session. The first administration of 5 mg/kg oxa-noribogaine reduced intake with a slight increase observed the following session. The second and third administration of 5 mg/kg oxa-noribogaine reduced intake in the first sessions with negligible increases the following day. Repeated intermittent dosing of 10 mg/kg and 5 mg/kg of oxa-noribogaine led to progressively increasing efficacy of morphine intake on the off days in the absence of oxa-noribogaine acute effects. Morphine intake increased from approximately 8% of baseline levels to only 40%—eighteen days following the last administration of 5 mg/kg oxa-noribogaine—showing effects that last far beyond the oxa-noribogaine exposure after this sub-chronic dosing regimen.

### Experiment 4: Effect of Repeated Administration of Oxa-Noribogaine on Fentanyl Intake and Fentanyl-Induced Hyperalgesia

The subjects, lever training and surgical procedures were identical to Experiment 1. Following recovery from surgery, rats were transferred to their respective operant chambers for daily self-administration sessions. Before each session, the swivel and catheter were flushed with heparinized saline before connecting the entry port of the swivel to the syringe mounted on the syringe pump outside of the sound attenuating chamber. For self-administration training, the stimulus light above the active lever was illuminated and both the active and inactive levers were extended. A response on the active lever (FR1) resulted in a 20 sec time out (FR1:TO 20 sec) during which time the subject received a 200 μl intravenous infusion (over the first six seconds), lever light extinguished, levers are retracted, tone is generated, and the house light is illuminated. At the end of the TO, the levers are extended, lever light illuminated, tone silenced and the house light extinguished (McIntosh, S. et al., 2015; Hemby, S. E. et al., 1999; Hemby, S. E. et al., 1996). Rats were trained initially to self-administer 1.25 mg/infusion of fentanyl for three days followed by 0.625 mg/infusion for 2 hours or 50 infusions. Responses on the inactive lever are recorded but have no adverse effect.

Progressive ratio procedure: Rats were allowed to self-administer fentanyl under a progressive ratio schedule of reinforcement, as described previously (Richardson, N. R., and Roberts, D. C., 1996), for three consecutive sessions. Fentanyl self-administration was maintained using the progressive ratio series 1, 2, 4, 6, 9, 12, 15, 20, 25, 32, 40, 50, 62, 77, 95, 118, 145, 178, 219, 268, 328, 402, 492, 603 derived from the following equation: =[5e^{(infusion number×2)}]−5. When the response requirement is met, a 20 sec time out is initiated, the subject receives an infusion subject received a 200 μl intravenous infusion (over the first six seconds), lever light is extinguished, levers are retracted, tone is generated, and the house light is illuminated. At the end of the TO, the levers are extended, lever light illuminated, tone is silenced, the house light is extinguished, and the program sets the current PR value to the next value in the series. Responses on the inactive lever are recorded but have no adverse effect. The sessions ends after 2 hours or after one hour elapsed with no responding on the active lever.

Electronic Von Frey Assessments: Mechanical sensitivity was measured using the electronic Von Frey 5 with embedded camera (BIO-EVF5; Bioseb US, Pinellas Park, FL). Rats were placed in a modular holder cage (BIO-PVF, Bioseb US) on a wire mesh elevated stand and allowed to acclimate for 5 min prior to assessment. The spring tip of the device was applied to the plantar surface of the hind paw. Brisk paw withdrawal was considered as a positive response the force required to initiate paw withdrawal was recorded by the EVF software. The average of a minimum of three replicates per hind paw was calculated for each rat and considered the paw withdrawal threshold.

Subjects were randomly assigned to one of two groups to receive either repeated vehicle (VEH) or oxa-noribogaine (Oxa-noriboga). An intermittent access (IntA) self-administration procedure, previously published by the Aston-Jones laboratory (James, M. H. et al., 2019; Fragale, J. E. et al., 2021), was used. Each daily 6-hr session consisted of 5-min bins of drug access separated by 25 minutes in which drug was not available for a total of one hour of drug access. Prior to each 5-min bin, rats received a priming infusion of fentanyl (208 ng/66.7 ml delivered over 2.08 seconds) paired with illumination of the lever light and a tone. For the 5 min bin, the house light was illuminated, and the active and inactive levers were extended. An active lever press made during the 5-min bin resulted in a fentanyl infusion (0.625 μg/200 ml over 6.17 seconds) paired with the lever light and tone. After the infusion period, the light was extinguished, and tone silenced. At the end of the 5-min bin, levers were retracted, and the house light was extinguished. The first session of IntA was designated as Session 1. Oxa-noribogaine was dissolved in 2 molar equivalents of acetic acid in water immediately prior to administration and administered in a volume of 2 ml/kg.

Following fentanyl self-administration training, the progressive ratio procedure was implemented to determine baseline reinforcing efficacy of fentanyl (Days 1-3). Von Frey assessments were conducted on prior to sessions. IntA self-administration was conducted for the remainder of the experiment with the exception of progressive ratio procedures conducted on days 16-18 and 50-51. Oxa-noribgoaine (40 mg/kg, IP) or vehicle were administered on Day 21 followed five days later by a series of injections on Days 26, 28, 30, 32, 34 and 36 of 10 mg/kg oxa-noribogaine or vehicle. IntA self-administration sessions were conducted on Days 37-54 with the exception of Days 50 and 51 when sessions consisted on progressive ratio procedure. A challenge dose of 10 mg/kg oxa-noribogaine or vehicle was administered on Day 52.

### Experiment 5: Effect of Repeated Administration of Oxa-Noribogaine and Vehicle on Food Maintained Responding

The subjects and lever training were identical to Experiment 1. For food maintained responding experiments food was restricted such that rats were maintained at 90% of their normal body weight until responding stabilized at which time rats were provided 12-14 g of rodent chow in addition to the food pellets earned during the experimental session. Food maintained responding after administration of vehicle or oxa-noribogaine (40 and 10 mg/kg doses) was determined using above described experimental sessions for lever training.

### Example 3. Tail-Flick Test

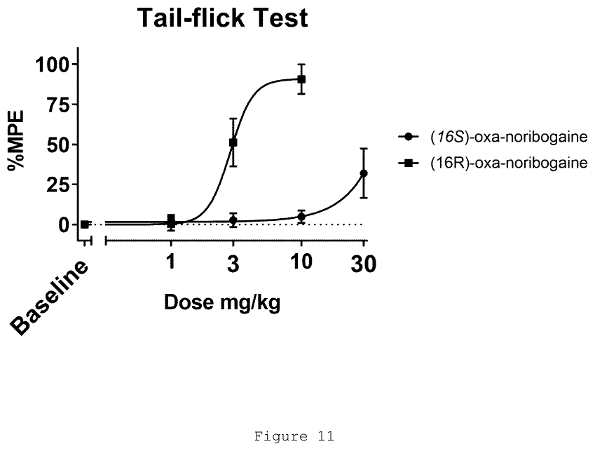

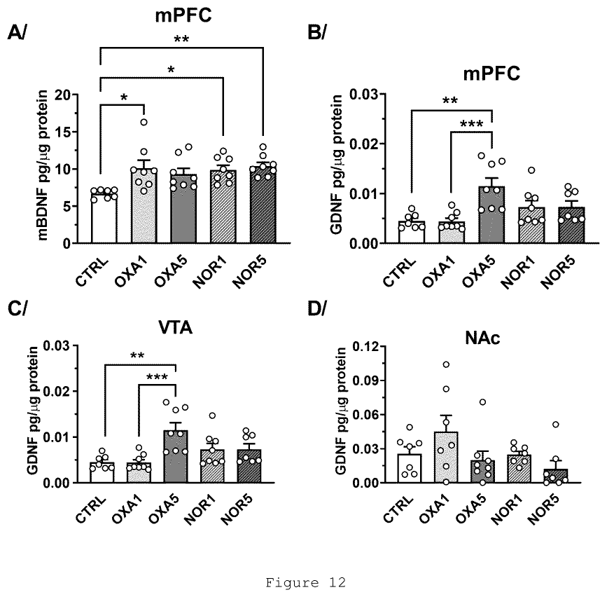

C57BL/6J (8-12 weeks, 22-31 g) were purchased from the Jackson Laboratory (Bar Harbor, ME) and housed 5 mice per cage with food and water available ad libitum. Mice were maintained on a 12-hr light/dark cycle (lights on 7:00-19:00) and all testing was done in the light cycle. Temperature was kept constant at 22±2° C., and relative humidity was maintained at 50±5%. Mice were moved to the testing room 30 minutes before the experiment to allow for acclimation. The body weight of each mouse and base tail-flick value were recorded. Mice were administered a 1 mg/kg s.c. dose of compound solution (volume of injection 220-310 μL based on body weight). After injection mice were returned to the home cage and allowed to rest for 30 minutes. Thirty minutes post injection the tail-flick measurement was taken using thermal stimulation via IR on a Ugo Basile unit set to 52 PSU (ten seconds was used as a maximum latency to prevent tissue damage). Mice were then administered 3 mg/kg s.c. dose, allowed to rest for 30 minutes, followed by another tail-flick measurement. This process was repeated for doses 10 and 30 mg/kg in increasing order. Tail-flick latencies for the different doses were expressed as percentage of maximum potential effect (% MPE) by subtracting the experimental value by the base tail flick value then dividing by the difference between the maximum possible latency (10 seconds) and the base tail-flick value and finally multiplying by 100. All tail flick experiments were performed by an experienced blinded male experimenter (FIG. 11).

### Example 4. Modulation of Neurotrophic Factor Expression Neurotropic Factors Expression Experiments (GDNF and Mature BDNF ELISAs)

Male Fisher F344 rats (Envigo) were decapitated, brains removed and placed in a stainless steel rat brain matrix. Coronal slices were taken and the ventral tegmental area (VTA), nucleus accumbens (NAc) and medial prefrontal cortex (mPFC) were dissected and immediately frozen on dry ice. Total protein was isolated from pulverized tissue from the ventral tegmental area, nucleus accumbens and medial prefrontal cortex from each subject. GDNF and mature BDNF were assayed using the BiosSensis GDNF, Rat, Rapid™ ELISA assay and the BDNF, mature, human, mouse, rat Rapid™ ELISA assay (Biosensis Pty Ltd, SA, Australia). Protease (Thermo Scientific, Rockford, IL), and phosphatase inhibitors (Cocktails 1 and 2, Sigma-Aldrich, St. Louis, MO) were added to the extraction buffers. Samples were sonicated twice for 15-20 seconds and incubated on ice for 30 seconds between sonication. Samples were then centrifuged at 15,000 rpm for 10 minutes at 4° C. and the supernatant (total protein lysate) was transferred to a new tube. Protein concentrations were measured using the bicinchoninic acid protein assay kit (Pierce, Rockford, IL, USA) on a spectrophotometer (iD5, Molecular Devices, Sunnyvale, CA). Aliquots of 100 μl of isolated protein from each region were transferred to a 96-well ELISA plates. Final absorbances were read at 450 nm using a spectrophotometer (iD5, Molecular Devices, Sunnyvale, CA). The abundance of GDNF and mBDNF were normalized to the amount of total protein (μg/mg protein) (FIG. 12).

### Example 5. Chiral Resolution of Ibogamine and Oxa-Noribogaine

Racemic material was analyzed and separated using supercritical fluid chromatography (SFC) on columns containing chiral stationary phase. Enantiomeric excess (ee) was determined by analytical SFC method and purity of racemate and each enantiomer was validated using RP-LC/MS (ACQUITY UPLC® instrument).

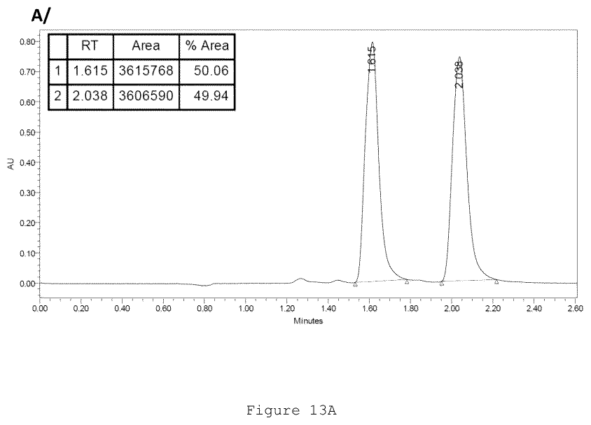

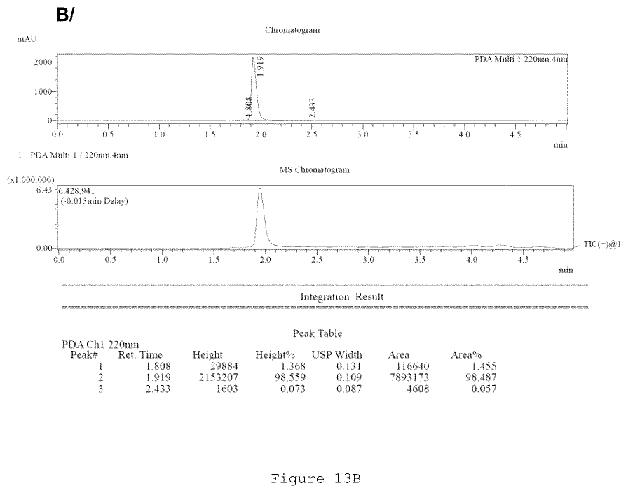

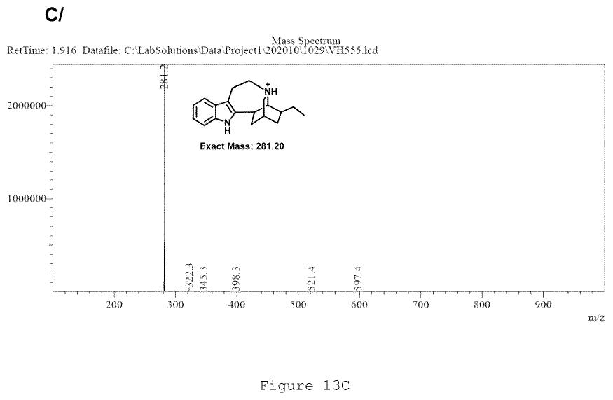

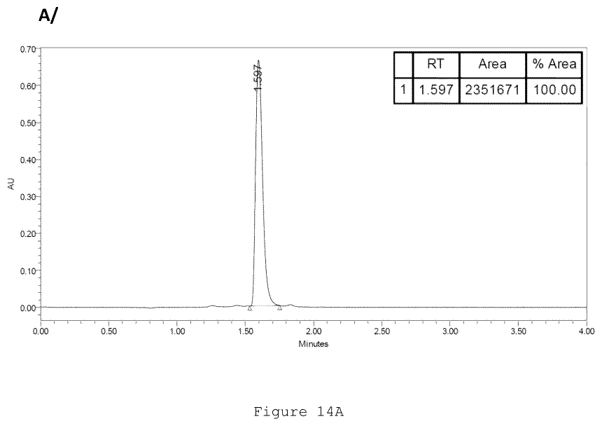

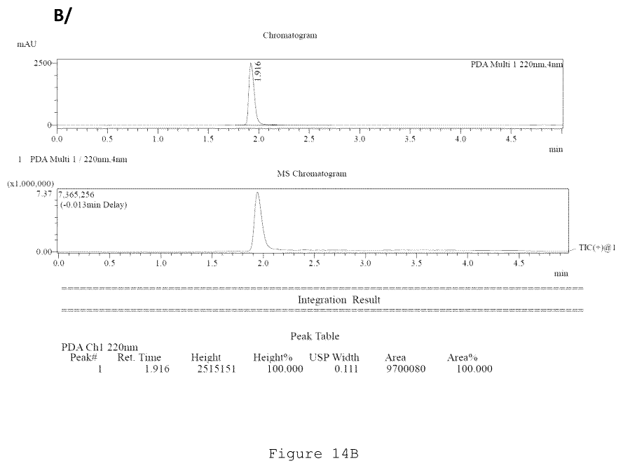

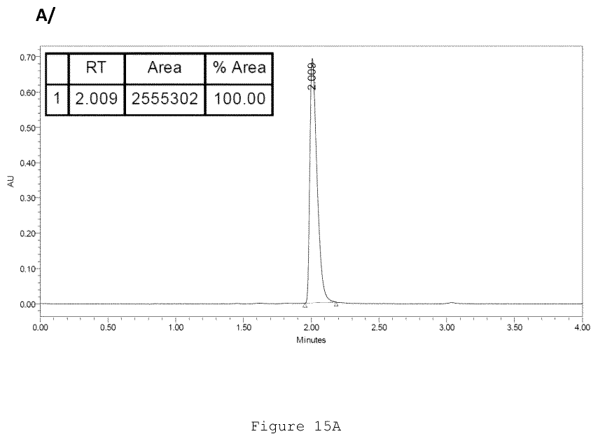

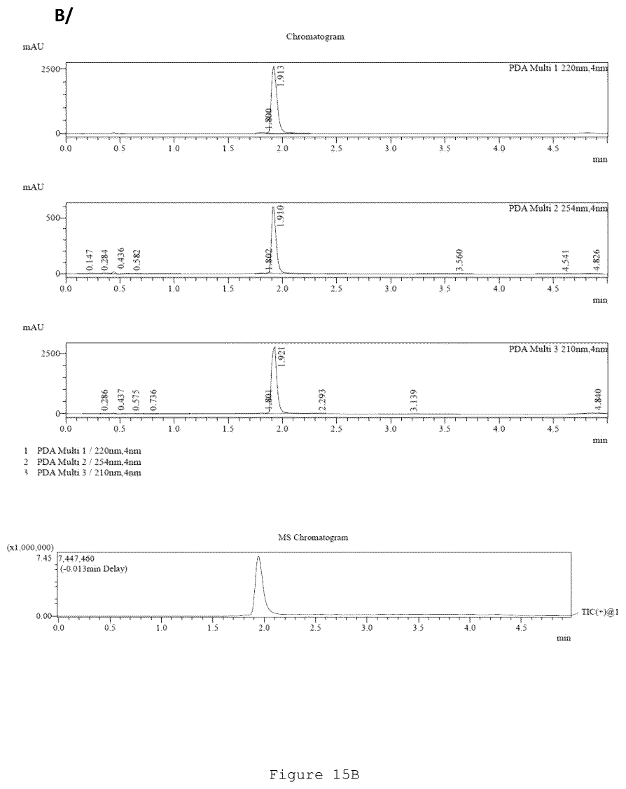

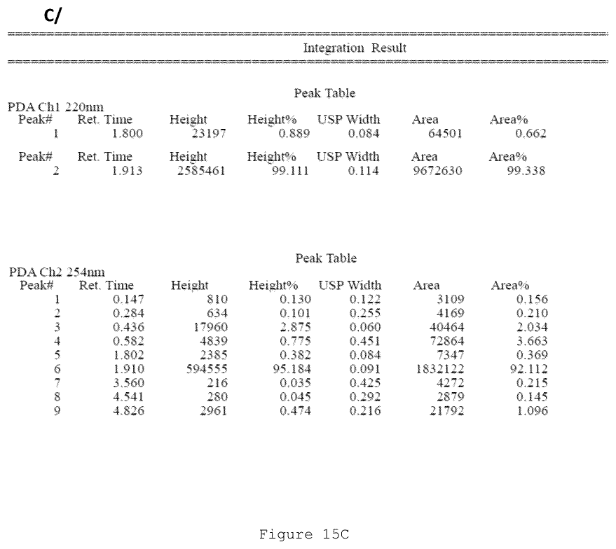

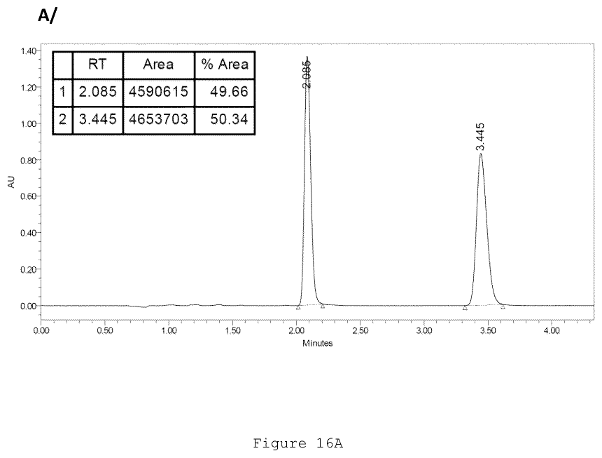

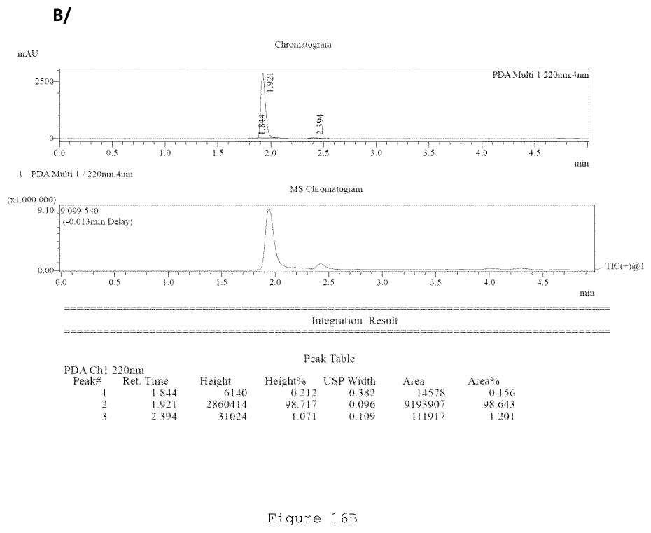

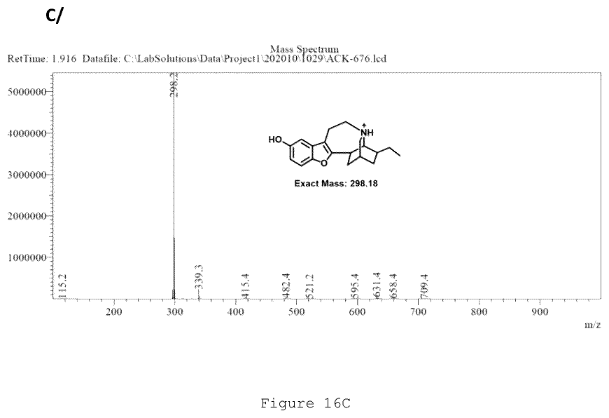

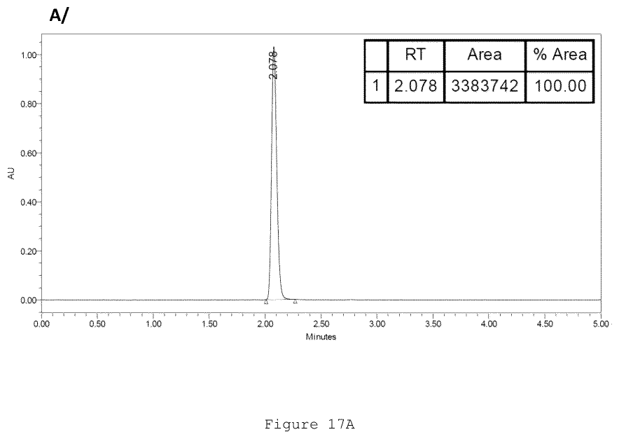

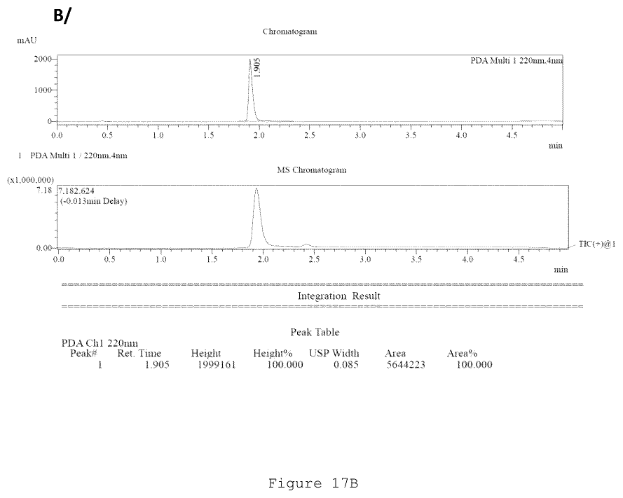

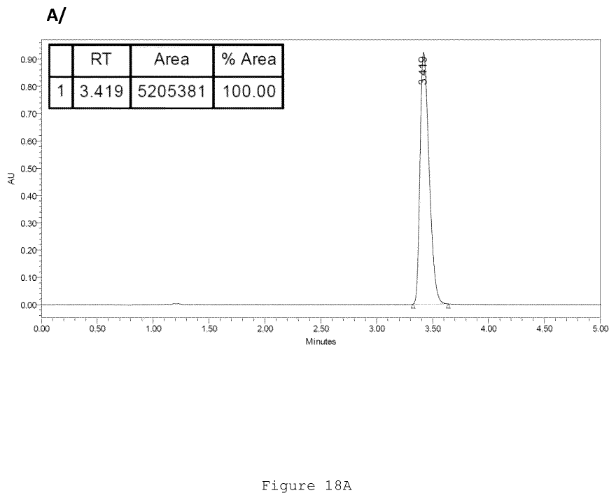

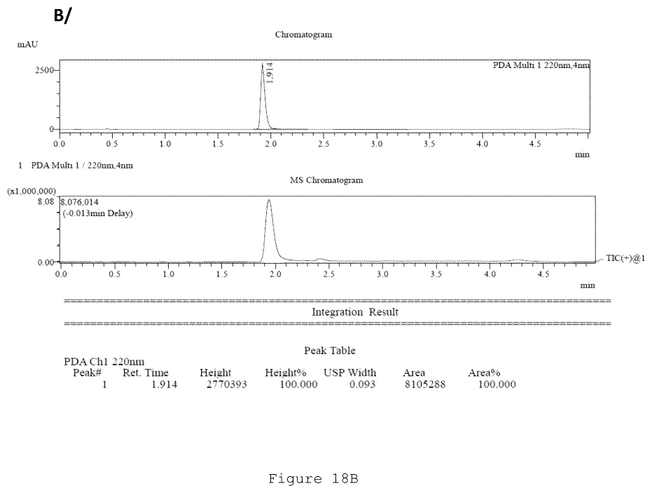

All 4 enantiomers (ibogamine and oxa-noribogaine, FIG. 19) were isolated in >99% ee purity, as determined by the analytical chiral SFC method (FIGS. 13-18).

TABLE 3
Analytical chiral separation method used for the
determination of enantiomeric excess (ee).

| Conditions | Ibogamine | Oxa-noribogaine |
|---|---|---|
| Instrument: | Waters UPC2 analytical SFC (PDA Detector) | |
| Flow rate: | 2.5 mL/min | |
| Column: | ChiralPak AD, 150 × 4.6 mm I.D., 3 μm | |
| Mobile phase: | A: CO₂ B: C₂H₅OH (0.05% Et₂NH) | |
| Gradient: | B: 40% | |
| Back pressure: | 100 bar | |
| Column | 35° C. | |
| temperature: | | |

| | |
|---|---|
| Wavelength: | 254 nm |

TABLE 4
Preparative separation method used.

| Conditions | Ibogamine | Oxa-noribogaine |
|---|---|---|
| Instrument | MG II preparative SFC | |
| Flow rate | 70 mL/min | |
| Column | ChiralPak AD, 250 × 30 mm I.D., 5 μm | |
| Mobile phase | A: CO₂ B: C₂H₅OH (0.1% of saturated NH₃ | |
| | solution in H₂O) | |
| Gradient | B: 40% | |
| Back pressure | 100 bar | |
| Column | 38° C. | |
| temperature | | |
| Wavelength | 220 nm | |
| Cycle time | ~3.6 min | ~8 min |
| Sample mass | 51 mg | 103 mg |
| Sample | Compound was dissolved | Compound was dissolved |
| preparation | in~10 ml CH₃OH/CH₂Cl₂ | in~20 ml CH₃OH/CH₂Cl₂ |
| Injection | 1 ml per injection | 3.5 ml per injection |

| | |
|---|---|
| Work up | After separation, the fractions were dried off via |
| | rotary evaporator at bath temperature 40° C. to get |
| | the desired isomers. |

### Example 6. Assignment of Oxa-Noribogaine Enantiomers by x-Ray Crystallography

Crystal Preparation

Oxa-noribogaine hydrochloride (2 mg) was dissolved in 200 μL of a mixture of methanol/methyl tert-butyl ether (1:1) and kept in a 1 mL tube. The solution was allowed to slowly evaporate at room temperature. Crystals were observed on the second day.

The crystal was a colourless block with the following dimensions: 0.10×0.10×0.04 mm³. The symmetry of the crystal structure was assigned the orthorhombic space group P212121 with the following parameters: a=7.14450(10) Å, b=12.62330(10) Å, c=18.2392(2) Å, α=90°, β=90°, γ=90°, V=1644.94(3) Å3, Z=4, Dc=1.348 g/cm³, F(000)=712.0, μ(CuKα)=2.126 mm⁻¹, and T=293(2) K.

Description of Equipment and Data Collection

Rigaku Oxford Diffraction XtaLAB Synergy four-circle diffractometer equipped with a HyPix-6000HE area detector.
Cryogenic system: Oxford Cryostream 800
Cu: λ=1.54184 Å, 50 W, Micro focus source with multilayer mirror (μ-CMF).
Distance from the crystal to the CCD detector: d=35 mm
Tube Voltage: 50 kV
Tube Current: 1 mA

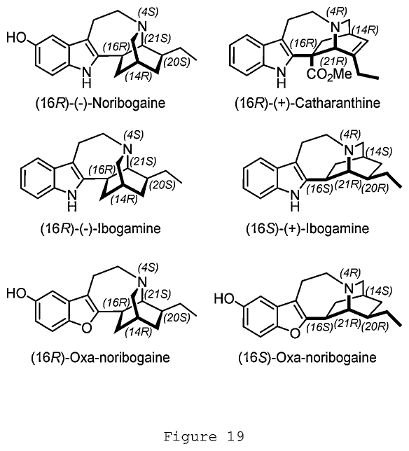

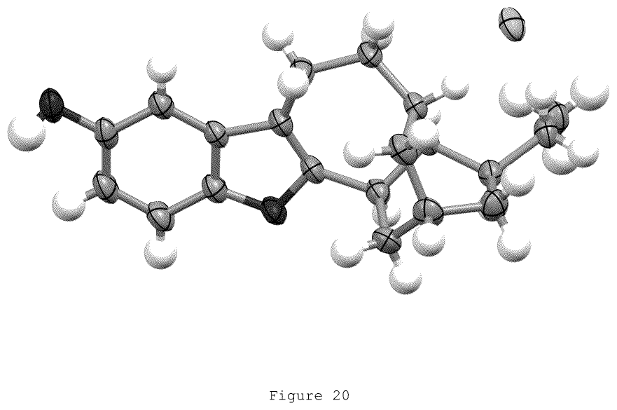

A total of 29322 reflections were collected in the 26 range from 8.518 to 133.15. The limiting indices were: −8≤h≤8, −15≤k≤14, −21≤l≤21; which yielded 2914 unique reflections (Rint=0.0346). The structure was solved using SHELXT (Sheldrick, G. M. 2015) and refined using SHELXL (against F²) (Sheldrick, G. M. 2015) (FIG. 20). The total number of refined parameters was 210, compared with 2914 data. All reflections were included in the refinement. The goodness of fit on F² was 1.049 with a final R value for [I>2σ(I)] R1=0.0242 and wR2=0.0656. The largest differential peak and hole were 0.12 and −0.15 Å-3, respectively.

TABLE 5
Summary of X-ray Crystallographic Data

| Identification code | WX-X01Y01A |
|---|---|
| Empirical formula | C₁₉H₂₄ClNO₂ |
| Formula weight | 333.84 |
| Temperature/K | 293(2) |
| Crystal system | orthorhombic |
| Space group | P2₁2₁2₁ |
| a/Å | 7.14450(10) |
| b/Å | 12.62330(10) |
| c/Å | 18.2392(2) |
| α/° | 90 |
| β/° | 90 |
| γ/° | 90 |
| Volume/Å³ | 1644.94(3) |
| Z | 4 |
| ρ_{calc}g/cm³ | 1.348 |
| μ/mm⁻¹ | 2.126 |
| F(000) | 712.0 |
| Crystal size/mm³ | 0.1 × 0.1 × 0.04 |
| Radiation | CuKα (λ = 1.54184) |
| 2Θ range for data | 8.518 to 133.15 |
| collection/° | |
| Index ranges | −8 ≤ h ≤ 8, −15 ≤ k ≤ 14, −21 ≤ l ≤ |
| | 21 |
| Reflections collected | 29322 |
| Independent reflections | 2914 [Rᵢₙₜ = 0.0346, R_{sigma} = 0.0133] |
| Data/restraints/parameters | 2914/0/210 |
| Goodness-of-fit on F² | 1.049 |
| Final R indexes [I >= 2σ (I)] | R₁ = 0.0242, wR₂ = 0.0656 |
| Final R indexes [all data] | R₁ = 0.0246, wR₂ = 0.0660 |
| Largest diff. peak/hole/e | 0.12/−0.15 |
| Å⁻³ | |
| Flack parameter | −0.009(4) |

TABLE 6
Atomic coordinates (×10\hat{ }4) and equivalent isotropic displacement
parameters (A\hat{ }2 × 10\hat{ }3).

| Atom | | x | | y | | z | | U(eq) | |
|---|---|---|---|---|---|---|---|---|---|
| Cl | (1) | 5760.1 | (7) | 2283.8 | (4) | 6742.0 | (3) | 47.81 | (15) |
| O | (2) | 1022.9 | (18) | 7431.3 | (10) | 6021.6 | (7) | 40.9 | (3) |
| O | (1) | 6663 | (2) | 10340.8 | (11) | 5730.3 | (8) | 49.5 | (4) |
| N | (1) | 3730 | (2) | 4398.0 | (12) | 6510.4 | (8) | 32.5 | (3) |
| C | (12) | 2114 | (3) | 6526.6 | (15) | 6108.3 | (10) | 35.2 | (4) |
| C | (10) | 1815 | (2) | 4506.6 | (14) | 6164.2 | (9) | 31.3 | (4) |
| C | (11) | 983 | (3) | 5590.0 | (14) | 6343.8 | (10) | 35.7 | (4) |
| C | (6) | 4093 | (3) | 7871.0 | (13) | 5906.4 | (9) | 35.1 | (4) |
| C | (1) | 5588 | (3) | 8568.8 | (14) | 5833.6 | (10) | 38.3 | (4) |
| C | (5) | 2264 | (3) | 8248.4 | (14) | 5908.5 | (11) | 38.7 | (4) |
| C | (7) | 3948 | (3) | 6741.3 | (14) | 6035.2 | (10) | 35.2 | (4) |
| C | (2) | 5179 | (3) | 9643.3 | (15) | 5778.9 | (10) | 40.2 | (4) |
| C | (17) | 544 | (3) | 3644.3 | (14) | 6490.7 | (10) | 36.2 | (4) |
| C | (9) | 5294 | (3) | 4866.9 | (15) | 6071.3 | (12) | 42.4 | (5) |
| C | (4) | 1827 | (3) | 9309.5 | (16) | 5834.6 | (12) | 46.5 | (5) |
| C | (13) | 588 | (3) | 5620.8 | (16) | 7187.0 | (11) | 45.4 | (5) |
| C | (14) | 1635 | (3) | 4715.2 | (17) | 7555.2 | (10) | 45.7 | (5) |
| C | (16) | 791 | (4) | 3652.0 | (16) | 7330.7 | (11) | 48.3 | (5) |
| C | (19) | −32 | (3) | 2443.8 | (18) | 5400.5 | (11) | 51.1 | (5) |
| C | (18) | 793 | (3) | 2542.7 | (14) | 6158.7 | (10) | 41.5 | (4) |
| C | (8) | 5615 | (3) | 6050.1 | (15) | 6149.6 | (12) | 42.6 | (4) |
| C | (15) | 3669 | (3) | 4747.6 | (17) | 7308.7 | (10) | 42.6 | (5) |
| C | (3) | 3333 | (3) | 10000.1 | (16) | 5769.4 | (12) | 48.0 | (5) |

TABLE 7
Bond lengths [A].

| Atom | Atom | Length/Å | Atom | Atom | Length/Å |
|---|---|---|---|---|---|
| O(2) | C(12) | 1.392(2) | C(6) | C(7) | 1.449(2) |
| O(2) | C(5) | 1.376(2) | C(1) | C(2) | 1.391(3) |
| O(1) | C(2) | 1.381(3) | C(5) | C(4) | 1.382(3) |
| N(1) | C(10) | 1.513(2) | C(7) | C(8) | 1.491(3) |
| N(1) | C(9) | 1.497(2) | C(2) | C(3) | 1.394(3) |
| N(1) | C(15) | 1.522(2) | C(17) | C(16) | 1.542(3) |
| C(12) | C(11) | 1.495(3) | C(17) | C(18) | 1.527(2) |
| C(12) | C(7) | 1.344(3) | C(9) | C(8) | 1.518(3) |
| C(10) | C(11) | 1.527(2) | C(4) | C(3) | 1.390(3) |
| C(10) | C(17) | 1.538(3) | C(13) | C(14) | 1.522(3) |
| C(11) | C(13) | 1.564(3) | C(14) | C(16) | 1.527(3) |
| C(6) | C(1) | 1.391(3) | C(14) | C(15) | 1.521(3) |
| C(6) | C(5) | 1.391(3) | C(19) | C(18) | 1.508(3) |

TABLE 8
Bond angles [deg].

| Atom | Atom | Atom | Angle/° | Atom | Atom | Atom | Angle/° |
|---|---|---|---|---|---|---|---|
| C(5) | O(2) | C(12) | 105.75(14) | C(12) | C(7) | C(6) | 106.53(17) |
| C(10) | N(1) | C(15) | 110.31(14) | C(12) | C(7) | C(8) | 130.28(17) |
| C(9) | N(1) | C(10) | 114.58(14) | C(6) | C(7) | C(8) | 122.77(18) |
| C(9) | N(1) | C(15) | 114.74(16) | O(1) | C(2) | C(1) | 117.71(19) |
| O(2) | C(12) | C(11) | 112.26(16) | O(1) | C(2) | C(3) | 121.31(17) |
| C(7) | C(12) | O(2) | 111.66(16) | C(1) | C(2) | C(3) | 120.97(19) |
| C(7) | C(12) | C(11) | 135.61(17) | C(10) | C(17) | C(16) | 108.22(15) |
| N(1) | C(10) | C(11) | 110.10(14) | C(18) | C(17) | C(10) | 114.97(15) |
| N(1) | C(10) | C(17) | 107.96(14) | C(18) | C(17) | C(16) | 112.72(16) |
| C(11) | C(10) | C(17) | 108.72(15) | N(1) | C(9) | C(8) | 116.83(16) |
| C(12) | C(11) | C(10) | 115.87(15) | C(5) | C(4) | C(3) | 116.2(2) |
| C(12) | C(11) | C(13) | 111.12(16) | C(14) | C(13) | C(11) | 109.04(16) |
| C(10) | C(11) | C(13) | 107.67(15) | C(13) | C(14) | C(16) | 110.33(18) |
| C(1) | C(6) | C(5) | 120.33(17) | C(15) | C(14) | C(13) | 108.60(18) |
| C(1) | C(6) | C(7) | 133.94(19) | C(15) | C(14) | C(16) | 108.75(18) |
| C(5) | C(6) | C(7) | 105.61(17) | C(14) | C(16) | C(17) | 108.49(16) |
| C(6) | C(1) | C(2) | 117.58(19) | C(19) | C(18) | C(17) | 113.17(16) |
| O(2) | C(5) | C(6) | 110.43(15) | C(7) | C(8) | C(9) | 116.25(17) |
| O(2) | C(5) | C(4) | 126.58(19) | C(14) | C(15) | N(1) | 107.59(15) |
| C(4) | C(5) | C(6) | 122.94(19) | C(4) | C(3) | C(2) | 121.94(18) |

TABLE 9
Hydrogen Bonds.

| D | H | A | d(D − H)/Å | d(H − A)/Å | d (D − A)/Å | D − H − A/° |
|---|---|---|---|---|---|---|
| O(1) | H(1) | Cl(1)¹ | 0.82 | 2.32 | 3.1364(15) | 173.8 |
| N(1) | H(1A) | Cl(1) | 0.98 | 2.16 | 3.0667(15) | 152.5 |

¹+ X, 1 + Y, +Z

TABLE 10
Torsion angles [deg].

| A | B | C | D | Angle/° | A | B | C | D | Angle/° |
|---|---|---|---|---|---|---|---|---|---|
| O(2) | C(12) | C(11) | C(10) | 156.47(15) | C(1) | C(6) | C(5) | O(2) | −177.11(16) |
| O(2) | C(12) | C(11) | C(13) | −80.24(19) | C(1) | C(6) | C(5) | C(4) | 0.5(3) |
| O(2) | C(12) | C(7) | C(6) | 1.3(2) | C(1) | C(6) | C(7) | C(12) | 175.4(2) |
| O(2) | C(12) | C(7) | C(8) | 173.75(18) | C(1) | C(6) | C(7) | C(8) | 2.2(3) |
| O(2) | C(5) | C(4) | C(3) | 176.13(19) | C(1) | C(2) | C(3) | C(4) | 1.9(3) |
| O(1) | C(2) | C(3) | C(4) | −178.50(19) | C(5) | O(2) | C(12) | C(11) | 171.75(15) |
| N(1) | C(10) | C(11) | C(12) | 58.1(2) | C(5) | O(2) | C(12) | C(7) | −1.6(2) |
| N(1) | C(10) | C(11) | C(13) | −67.02(19) | C(5) | C(6) | C(1) | C(2) | 1.2(3) |
| N(1) | C(10) | C(17) | C(16) | 47.2(2) | C(5) | C(6) | C(7) | C(12) | −0.4(2) |
| N(1) | C(10) | C(17) | C(18) | −79.82(18) | C(5) | C(6) | C(7) | C(8) | −173.59(18) |
| N(1) | C(9) | C(8) | C(7) | −52.3(3) | C(5) | C(4) | C(3) | C(2) | −0.1(3) |
| C(12) | O(2) | C(5) | C(6) | 1.3(2) | C(7) | C(12) | C(11) | C(10) | −32.3(3) |
| C(12) | O(2) | C(5) | C(4) | −176.2(2) | C(7) | C(12) | C(11) | C(13) | 91.0(3) |
| C(12) | C(11) | C(13) | C(14) | −112.49(19) | C(7) | C(6) | C(1) | C(2) | −174.1(2) |
| C(12) | C(7) | C(8) | C(9) | 24.7(3) | C(7) | C(6) | C(5) | O(2) | −0.6(2) |
| C(10) | N(1) | C(9) | C(8) | 82.8(2) | C(7) | C(6) | C(5) | C(4) | 177.04(19) |
| C(10) | N(1) | C(15) | C(14) | 19.5(2) | C(17) | C(10) | C(11) | C(12) | 176.13(15) |
| C(10) | C(11) | C(13) | C(14) | 15.4(2) | C(17) | C(10) | C(11) | C(13) | 51.05(19) |
| C(10) | C(17) | C(16) | C(14) | 19.2(2) | C(9) | N(1) | C(10) | C(11) | −83.84(17) |
| C(10) | C(17) | C(18) | C(19) | −73.9(2) | C(9) | N(1) | C(10) | C(17) | 157.61(15) |
| C(11) | C(12) | C(7) | C(6) | −170.0(2) | C(9) | N(1) | C(15) | C(14) | 150.73(17) |
| C(11) | C(12) | C(7) | C(8) | 2.5(4) | C(13) | C(14) | C(16) | C(17) | 47.6(2) |
| C(11) | C(10) | C(17) | C(16) | −72.23(19) | C(13) | C(14) | C(15) | N(1) | −71.5(2) |
| C(11) | C(10) | C(17) | C(18) | 160.76(16) | C(16) | C(17) | C(18) | C(19) | 161.46(18) |
| C(11) | C(13) | C(14) | C(16) | −68.3(2) | C(16) | C(14) | C(15) | N(1) | 48.6(2) |
| C(11) | C(13) | C(14) | C(15) | 50.8(2) | C(18) | C(17) | C(16) | C(14) | 147.52(18) |
| C(6) | C(1) | C(2) | O(1) | 177.96(16) | C(15) | N(1) | C(10) | C(11) | 47.47(19) |
| C(6) | C(1) | C(2) | C(3) | −2.4(3) | C(15) | N(1) | C(10) | C(17) | −71.08(18) |
| C(6) | C(5) | C(4) | C(3) | −1.1(3) | C(15) | N(1) | C(9) | C(8) | −46.3(2) |
| C(6) | C(7) | C(8) | C(9) | −163.93(18) | C(15) | C(14) | C(16) | C(17) | −71.4(2) |

### Example 7. Circular Dichroism (CD) Spectra

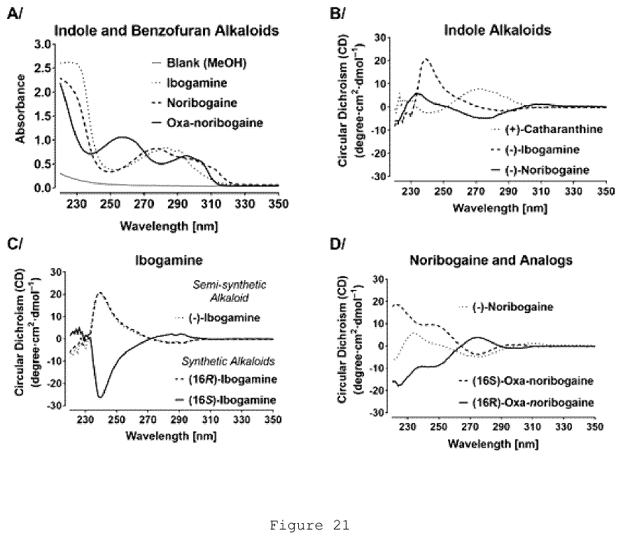

Novel compounds, natural and semi-synthetic alkaloids were recorded using Chirascan™ V100 Spectrometer at room temperature (25-30° C.) using reduced volume 10 mm quartz cuvettes. Samples were dissolved in HPLC grade methanol (concentration 0.1 mM) and were measured against air set as background (FIG. 21).

TABLE 11
Retention times and optical rotation of oxa-
noribogaine and ibogamine enantiomers.

| Compound | Retention time (min) | Optical rotation |
|---|---|---|
| Oxa-noribogaine (16S) | 2.085 | 73.42 |
| Oxa-noribogaine (16R) | 3.445 | −69.81 |
| Ibogamine (16S) | 2.038 | 52.40 |
| Ibogamine (16R) | 1.615 | −49.16 |

### Example 8. Pharmacological Characterization of Oxa-Noribogaine

EC₅₀ values and % efficacy of control agonist (KOR: U-50,488) maximal response at 1 μM for rKOR-CHO were obtained by screening the selected compounds via commercially available assay services.

Inhibition assays of transporters (hSERT and rVMAT2) were performed according to the following protocol.

Cell culture preparation and maintenance. Stably transfected hSERT-HEK and rVMAT2-HEK cellular cultures were maintained in Dulbecco's Minimal Essential Medium (DMEM) with GlutaMAX (Gibco) with the following additions: 10% (v/v) Fetal Bovine Serum (FBS, Atlanta Biologicals), 100 U/mL Penicillin (Gibco), and 10 μg/mL Streptomycin (Gibco). With regards to the former cell lineage, an additional ingredient, 500 μg/mL Geneticin (G418) (Gibco) was included to preserve the respective transgene.

hSERT and rVMAT2 fluorometric screening assays. For both hSERT and rVMAT2 screening experiments, respective singly transfected cells were seeded at a density of 0.09×10⁶ cells/well in poly-D-Lysine (Alamanda Polymers, Inc.) coated white solid-bottom 96-well plates (Costar). Growth was permitted for approximately 44 hours in said aqueous media and at an incubation environment of 37° C. and 5% Carbon Dioxide. At the beginning of the experiment, the cellular growth solution was aspirated, and individual cells were rinsed with 150 μL of 1×Dulbecco's Phosphate Buffered Saline (PBS; HyClone). 63 μL of Experimental Media (consisting of the following contents: DMEM without phenol red but with 4.5 g/L of D-Glucose (Gibco), 1% (v/v) FBS (Atlanta Biologicals), 100 U/mL Penicillin (Gibco), and 10 μg/mL Streptomycin (Gibco)) with 2×tiered concentrations of inhibitor (or DMSO, the vehicle of these experiments) were added to the respective wells. Control inhibitors used in these studies include Imipramine for hSERT experiments, and Reserpine for rVMAT2 experiments (Eiden, L. E. and Weihe, E. 2011; Sette, M. et al. 1983). At the conclusion of the pre-incubation period (60 minutes for hSERT experiments and 30 minutes for rVMAT2 experiments), 63 μL of Experimental Media containing 2×various concentrations of tested inhibitor (or vehicle) along with a specified amount of fluorescent substrate, APP+(Karpowicz, R. J. et al 2013) (final concentration: 1.1 μM for hSERT experiments) or FFN206 (Hu, G. et al. 2013) (final concentration: 0.75 μM for rVMAT2 experiments) were added to the present solution contained within the wells. After a required incubation period (30 minutes for hSERT experiments and 60 minutes for rVMAT2 experiments) for proper fluorescent probe uptake, the contents of each well were aspirated and consequently, rinsed twice with 120 μL of PBS. A final solution of 120 μL of PBS is finally added to all corresponding wells for cell maintenance before undergoing fluorescence uptake reading by a BioTek H1MF plate reader. The excitation and emission wavelengths of APP⁺ were set at 389 and 442 nm, respectively. Alternatively, the excitation and emission wavelengths of FFN206 were designed at 370 and 464 nm, respectively.

Data analysis. Numerical analysis of the collected experimental data preceded as accordingly. Respective inhibitor values were first subtracted from vehicular values to quantify the respective fluorescence uptake. This metric was then analyzed using the dose-response-inhibitor nonlinear curve fitting model ([inhibitor] vs response (three parameters)) as supplied by GraphPad Prism 8 software. For each inhibitor, the model supplied a respective IC₅₀±SEM value (Table 12). From this intermediate metric, calculation of the inhibition constant, Kᵢ±SEM, was made possible using the Cheng-Prusoff Equation (Yung-Chi, C. and Prusoff, W. H. 1973) and the following established constants: Kₘ (for APP⁺)=1.6 μM (hSERT) and Kₘ (for FFN206)=1.2 μM (rVMAT2). It must be noted that the lower the Kᵢ value that is found, the greater the potency that the candidate inhibitor possesses at said transporter.

TABLE 12
IC₅₀ values for hSERT and rVMAT2 transporter inhibition
assays and EC₅₀ values for rKOR agonist assays (cAMP).
For KOR in parenthesis are indicated % efficacy of control
agonist (KOR: U-50,488) maximal response at 1 μM.

| | hSERT (IC₅₀) | rVMAT2 (IC₅₀) | rKOR (EC₅₀) | |
|---|---|---|---|---|
| Compound | [μM ± SEM] | [μM ± SEM] | [μM] | |
| Oxa-noribogaine | 0.90 ± 0.12 | 0.67 ± 0.11 | <<0.01 | (102%) |
| (racemic) | | | | |
| (16S)-Oxa- | 2.9 ± 0.68 | 2.8 ± 0.55 | ~0.3 | (25%) |
| noribogaine | | | | |
| (16R)- Oxa- | 0.45 ± 0.07 | 0.14 ± 0.03 | <<0.01 | (96%) |

noribogaine

### DISCUSSION

As described in the Experimental section, oxa-iboga compounds attenuate intake of morphine, fentanyl, and cocaine in a well established model of SUDs, rat self-administration paradigm (Katz, J. L. 1989; Lynch W. J. & Hemby, S. E. 2011). In addition to acute effects, we found that the suppression of drug intake lasts for several days after a single administration of oxa-noribogaine or its analogs. We also found that a regimen of repeated dosing of this compound profoundly reduces morphine intake for at least eighteen days after the last dose of oxa-iboga analog (far beyond the drug exposure period). Additionally, we found that a regimen of repeated dosing of oxa-noribogaine reduces fentanyl intake and alleviates fentanyl-induced hyperalgesia in a model of severe-fentanyl addiction states. These lasting effects are drug selective as no such long effects were seen in food responding (i.e. operant behavior induced by natural rewards). These results suggest desirable persistent neuroplasticity and neuro-restorative effects of oxa-iboga compounds on relevant brain circuitry. As such these compounds represent important candidates for new SUD pharmacotherapeutics.

Further, we discovered that oxa-iboga analogs have much improved safety profile in terms of cardiac adverse effects as compared to noribogaine (a long lasting metabolite of ibogaine). Using adult human primary cardiomyocytes, in a state-of-the-art essay with high predictive validity of clinical cardiac effects, we found that while noribogaine exhibits pro-arrhytmia effects, oxa-noribogaine and other oxa-iboga compounds show no pro-arrhytmia effects even at high concentrations (e.g. 30-fold over the expected free therapeutic plasma concentrations of these compounds).

As part of the continuing research on these compounds (U.S. application Ser. No. 15/528,339; PCT International Application No. PCT/US2015/062726), we found that enantiomers of oxa-noribogaine have very different potency at KOR and other molecular targets (Table 12).

### REFERENCES

Alper, K. R. Alkaloids Chem Biol 56, 1-38 (2001).
Alper, K. et al. Cardiovasc Toxicol 16, 14-22 (2016).
Belgers, M. et al. Transl Psychiatry 6, e826 (2016).
Bolan, E. A. et al. Synapse 51, 11-8 (2004).
Eiden, L. E. & Weihe, E. Annals of the New York Academy of Sciences 1216, 86-98 (2011).
Fragale, J. E. et al. Addict. Biol. 26, e12946 (2021).
Glick, S. D. et al. Alkaloids Chem Biol 56, 39-53 (2001).
Glue, P. et al. Clinical Pharmacology in Drug Development 5, 460-468 (2016).
Hemby, S. E. et al. J. Pharmacol. Exp. Ther. 277, 1247-1258 (1996).
Hemby, S. E. et al. J. Pharmacol. Exp. Ther. 288, 274-280 (1999).
Hu, G. et al. ACS Chemical Biology 8, 1947-1954 (2013).
James, M. H. et al. Biol. Psychiatry 85, 925-935 (2019).
Karpowicz, R. J. et al. ACS Chemical Neuroscience 4, 858-869 (2013).
Katz, J. L. Drugs as reinforcers: pharmacological and behavioural factors. In: Liebman J M, Cooper S J eds. The Neuropharmacological Basis of Reward, pp. 164-213. Oxford University Press: New York (1989).
Koenig, X. & Hilber, K. Molecules 20, 2208-2228 (2015).
Kruegel, A. C. et al. Journal of the American Chemical Society, 138, 6754-6764 (2016).
Lynch, W. J. & Hemby, S. E. Drug reinforcement in animals. In: Johnson B A ed. Addiction Medicine: Science and Practice, pp. 117-128. Springer: New York (2011).
Majumdar, S. et al. Bioorg. Med. Chem. Lett. 21, 4001-4004 (2011).
Mash, D. C. et al. J Psychopharmacol 30, 688-697 (2016).
McIntosh, S. et al. J. Neuroimmune Pharmacol. 10, 493-505 (2015).
Nguyen, N. et al. Front. Physiol. 8, 1073 (2017).
Page, G. et al. J. Pharmacol. Toxicol. Methods 81, 183-195 (2016).
Pattison, L. P. et al. J. Neurochem. 122, 138-146 (2012).
Pattison, L. P. et al. Synapse 68, 437-444 (2014).
Redfern, W. S. et al. Cardiovascular Research 58, 32-45 (2003).
Richardson, N. R. & Roberts, D. C. S. J. Neurosci. Methods 66, 1-11 (1996).
Rubi, L. et al. Cardiovasc Toxicol 17, 215-218 (2017).
Sette, M. et al. Journal of Neurochemistry 40, 622-628 (1983).
Sheldrick, G. M. Acta Cryst. A 71, 3-8 (2015).
Yung-Chi, C. & Prusoff, W. H. Biochemical Pharmacology 22(23), 3099-3108 (1973).

What is claimed is:

1. A method of treating a subject afflicted with a substance use disorder (SUD) comprising administering to the subject an effective amount of a compound having the structure: 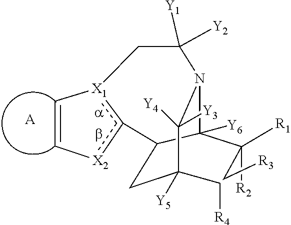
wherein
A is a ring structure, with or without substitution;
X₁ is C or N;
X₂ is N, O, or S;
Y₁ is H, -(alkyl), -(alkenyl), -(alkynyl), -(cycloalkyl), -(haloalkyl), -(alkyl)-O-(alkyl) or -(alkyl)-(cycloalkyl);
Y₂ is H, -(alkyl), -(alkenyl), -(alkynyl), -(cycloalkyl), -(haloalkyl), -(alkyl)-O-(alkyl) or -(alkyl)-(cycloalkyl);
Y₃ is H, -(alkyl), -(alkenyl), -(alkynyl), -(cycloalkyl), -(haloalkyl), -(alkyl)-O-(alkyl) or -(alkyl)-(cycloalkyl);
Y₄ is H, -(alkyl), -(alkenyl), -(alkynyl), -(cycloalkyl), -(haloalkyl), -(alkyl)-O-(alkyl) or -(alkyl)-(cycloalkyl);
Y₅ is H, -(alkyl), -(alkenyl), -(alkynyl), -(cycloalkyl), -(haloalkyl), -(alkyl)-O-(alkyl) or -(alkyl)-(cycloalkyl);
Y₆ is H, -(alkyl), -(alkenyl), -(alkynyl), -(cycloalkyl), -(haloalkyl), -(alkyl)-O-(alkyl) or -(alkyl)-(cycloalkyl);
α and β are each present or absent and when present each is a bond,
wherein either α or β is present, and
when α is present, then X₁ is C and X₂ is S or O, or
when β is present, then X₁ is N and X₂ is N; and
R₁, R₂, R₃ and R₄ are each independently —H, -(alkyl), -(alkenyl), -(alkynyl), -(haloalkyl), -(cycloalkyl), -(aryl), -(heteroaryl), -(heteroalkyl), -(hydroxyalkyl), -(alkyl)-(aryl), -(alkyl)- (heteroaryl), -(alkyl)-(cycloalkyl), -(alkyl)-OH, -(alkyl)-O-(alkyl), —OH, —NH₂, —OAc, —CO₂H, —CN, OCF₃, halogen, —CO₂—(C₂-C₁₂ alkyl), C(O)—NH₂, —C(O)—NH-(alkyl), C(O)—NH-(aryl), —O-alkyl, —O-alkenyl, —O-alkynyl, —O-aryl, —O-(heteroaryl), —NH-alkyl, —NH-alkenyl, —NH-alkynyl, —NH-aryl, —NH-(heteroaryl), —O—C(O) (alkyl), or —C(O)—N(alkyl)₂,
or a pharmaceutically acceptable salt or ester thereof, so as to thereby treat the subject afflicted with the substance use disorder (SUD).

2. The method of claim 1, wherein the substance use disorder is opioid use disorder, alcohol use disorder or stimulant use disorder including nicotine use disorder,
wherein the substance is an opioid,
wherein the opioid is morphine, hydromorphone, oxymorphone, codeine, dihydrocodeine, hydrocodone, oxycodone, nalbuphine, butorphanol, etorphine, dihydroetorphine, levorphanol, metazocine, pentazocine, meptazinol, meperidine (pethidine), buprenorphine, methadone, tramadol, tapentadol, mitragynine, 3-deutero-mitragynine, 7-hydroxymitragynine, 3-deutero-7-hydroxymitragynine, mitragynine pseudoindoxyl or tianeptine, or,
wherein the opioid is fentanyl, sufentanil, alfentanil, furanylfentanyl, 3-methylfentanyl, valerylfentanyl, butyrylfentanyl, R-Hydroxythiofentanyl, acrylfentanyl or carfentanil, or
wherein the stimulant is cocaine, amphetamine, methamphetamine, cathinone and its derivatives or nicotine,
wherein the risk of relapse to the use of opioids, alcohol or stimulants is reduced, or wherein self-administration of an opioid, alcohol or stimulant is reduced.

3-5. (canceled)

6. The method of claim 1, which comprises treating a symptom of substance use disorder
wherein a symptom of substance use disorder is opioid withdrawal, hyperalgesia or allodynia,
wherein the treating is effective for an extended time,
wherein the time is 1-5 days or 1-5 weeks, or
wherein the effective amount of the compound administered to the subject without inducing cardiotoxicity, without inducing QT interval prolongation or without inducing cardiac arrhythmia, or
wherein the subject is a mammal,
wherein the mammal is a human.

7-13. (canceled)

14. The method of claim 6, wherein the compound has the structure: 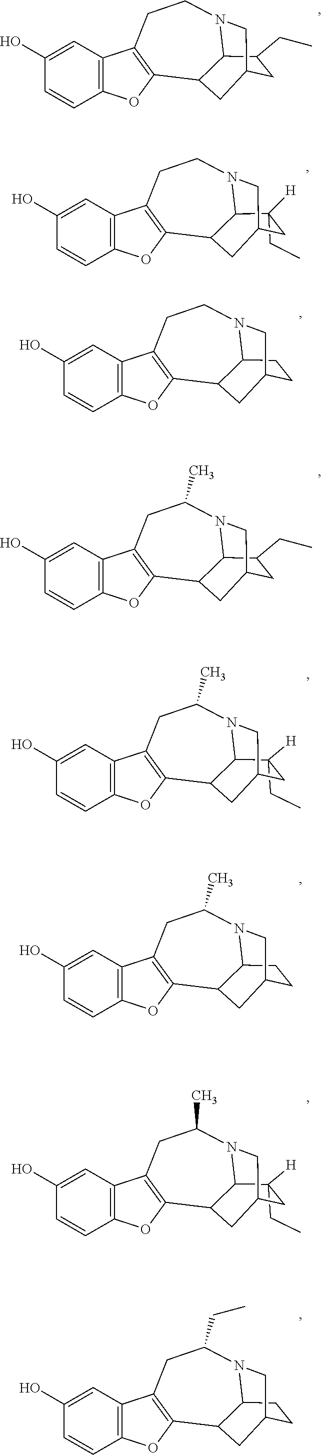 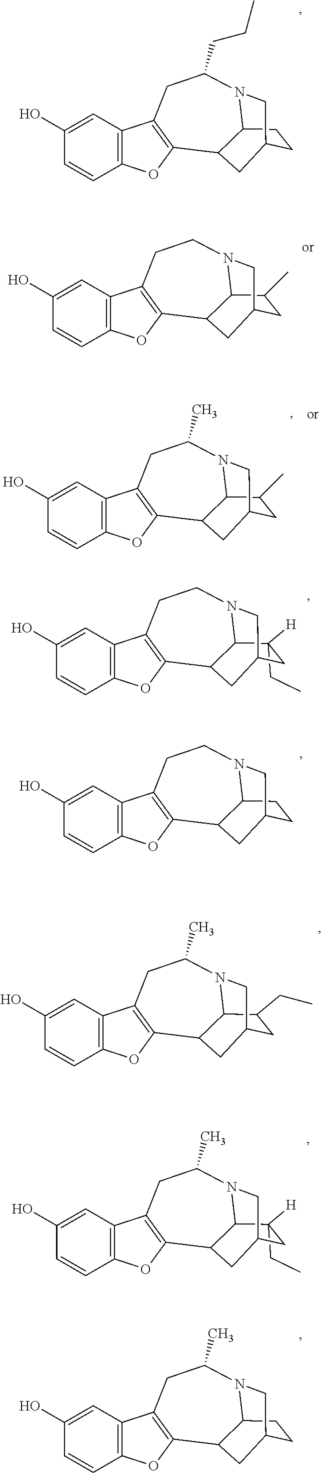 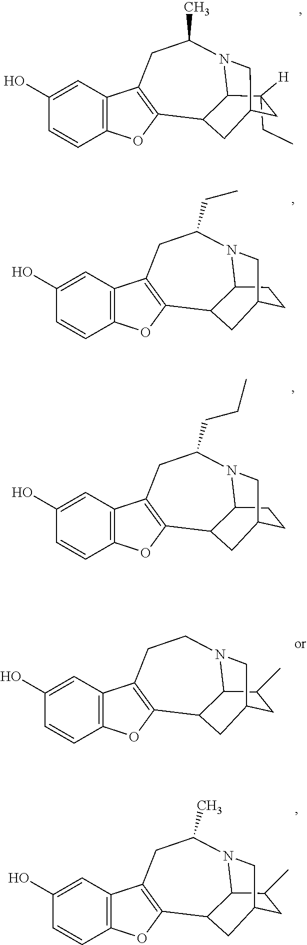 [SMILES: CCC1CC2CC3c4oc5ccc(O)cc5c4C[C@@H](C)N(C2)C13 ; CC[C@H]1Cc2c(oc3ccc(O)cc23)C2CC3CCC2N1C3 ; CCC[C@H]1Cc2c(oc3ccc(O)cc23)C2CC3CCC2N1C3 ; CC1CC2CC3c4oc5ccc(O)cc5c4CCN(C2)C13 ; CC1CC2CC3c4oc5ccc(O)cc5c4C[C@H](C)N(C2)C13]
or a pharmaceutically acceptable salt or ester thereof.

15. (canceled)

16. The method of claim 14, wherein the effective amount of 10-500 mg of the compound is administered to the subject.

17. The method of claim 16, comprising administering a pharmaceutical composition, which comprises the compound and a pharmaceutically acceptable carrier.

18. A compound having the structure: 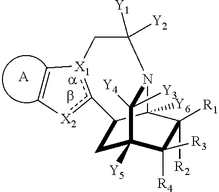
wherein
A is a ring structure, with or without substitution;
X₁ is C or N;
X₂ is N, O, or S;
Y₁ is H, -(alkyl), -(alkenyl), -(alkynyl), -(cycloalkyl), -(haloalkyl), -(alkyl)-O-(alkyl) or -(alkyl)-(cycloalkyl);
Y₂ is H, -(alkyl), -(alkenyl), -(alkynyl), -(cycloalkyl), -(haloalkyl), -(alkyl)-O-(alkyl) or -(alkyl)-(cycloalkyl);
Y₃ is H, -(alkyl), -(alkenyl), -(alkynyl), -(cycloalkyl), -(haloalkyl), -(alkyl)-O-(alkyl) or -(alkyl)-(cycloalkyl);
Y₄ is H, -(alkyl), -(alkenyl), -(alkynyl), -(cycloalkyl), -(haloalkyl), -(alkyl)-O-(alkyl) or -(alkyl)-(cycloalkyl);
Y₅ is H, -(alkyl), -(alkenyl), -(alkynyl), -(cycloalkyl), -(haloalkyl), -(alkyl)-O-(alkyl) or -(alkyl)-(cycloalkyl);
Y₆ is H, -(alkyl), -(alkenyl), -(alkynyl), -(cycloalkyl), -(haloalkyl), -(alkyl)-O-(alkyl) or -(alkyl)-(cycloalkyl);
α and β are each present or absent and when present each is a bond,
wherein either α or β is present, and
when α is present, then X₁ is C and X₂ is S or O, or
when β is present, then X₁ is N and X₂ is N; and
R₁, R₂, R₃ and R₄ are each independently —H, -(alkyl), -(alkenyl), -(alkynyl), -(haloalkyl), -(cycloalkyl), -(aryl), -(heteroaryl), -(heteroalkyl), -(hydroxyalkyl), -(alkyl)-(aryl), -(alkyl)- (heteroaryl), -(alkyl)-(cycloalkyl), -(alkyl)-OH, -(alkyl)-O-(alkyl), —OH, —NH₂, —OAc, —CO₂H, —CN, OCF₃, halogen, —CO₂—(C₂-C₁₂ alkyl), C(O)—NH₂, —C(O)—NH-(alkyl), C(O)—NH-(aryl), —O-alkyl, —O-alkenyl, —O-alkynyl, —O-aryl, —O-(heteroaryl), —NH-alkyl, —NH-alkenyl, —NH-alkynyl, —NH-aryl, —NH-(heteroaryl), —O—C(O) (alkyl), or —C(O)—N(alkyl)₂, or 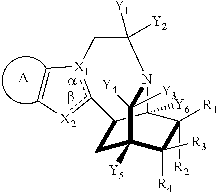
wherein
A is a ring structure, with or without substitution;
X₁ is C or N;
X₂ is N, O or S;
Y₁ is H, -(alkyl), -(alkenyl), -(alkynyl), -(cycloalkyl) or -(alkyl)-(cycloalkyl);
Y₂ is H, -(alkyl), -(alkenyl), -(alkynyl), -(cycloalkyl) or -(alkyl)-(cycloalkyl);
Y₃ is H, -(alkyl), -(alkenyl), -(alkynyl), -(cycloalkyl) or -(alkyl)-(cycloalkyl);
Y₄ is H, -(alkyl), -(alkenyl), -(alkynyl), -(cycloalkyl) or -(alkyl)-(cycloalkyl);
Y₅ is H, -(alkyl), -(alkenyl), -(alkynyl), -(cycloalkyl) or -(alkyl)-(cycloalkyl);
Y₆ is H, -(alkyl), -(alkenyl), -(alkynyl), -(cycloalkyl) or -(alkyl)-(cycloalkyl);
α and β are each present or absent and when present each is a bond,
wherein either α or β is present, and
when α is present, then X₁ is C and X₂ is S or O, or
when β is present, then X₁ is N and X₂ is N; and
R₁, R₂, R₃ and R₄ are each independently H, -(alkyl), -(alkenyl), -(alkynyl), -(aryl), -(heteroaryl), -(heteroalkyl), -(hydroxyalkyl), -(alkyl)-(aryl), -(alkyl)-(heteroaryl), -(alkyl)-OH, -(alkyl)- O-(alkyl), —OH, —NH₂, —CO₂H, —CO₂—(C₂-C₁₂ alkyl) or —C(O)—NH-(alkyl), or 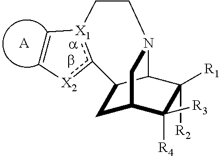
wherein
A is a ring structure, with or without substitution;
X₁ is C or N;
X₂ is N, O or S;
α and β are each present or absent and when present each is a bond,
wherein either α or β is present, and
when α is present, then X₁ is C and X₂ is S or O, or
when β is present, then X₁ is N and X₂ is N; and
R₁, R₂, R₃ and R₄ are each independently H, -(alkyl), -(alkenyl), -(alkynyl), -(aryl), -(heteroaryl), -(heteroalkyl), -(hydroxyalkyl), -(alkyl)-(aryl), -(alkyl)-(heteroaryl), -(alkyl)-OH, -(alkyl)- O-(alkyl), —OH, —NH₂, —CO₂H, —CO₂—(C₂-C₁₂ alkyl) or —C(O)—NH-(alkyl),
or a pharmaceutically acceptable salt or ester thereof.

19-20. (canceled)

21. The compound of claim 18 having the structure: 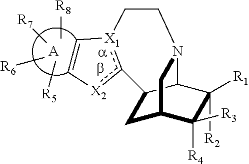
wherein
A is an aryl or heteroaryl;
X₁ is C or N;
X₂ is N, O or S;
α and β are each present or absent and when present each is a bond,
wherein either α or β is present,
when α is present, then X₁ is C and X₂ is S or O, and
when β is present, then X₁ is N and X₂ is N;
R₁, R₂, R₃ and R₄ are each independently H, -(alkyl), -(alkenyl), -(alkynyl), -(aryl), -(heteroaryl), -(heteroalkyl), -(hydroxyalkyl), -(alkyl)-(aryl), -(alkyl)-(heteroaryl), -(alkyl)-OH, -(alkyl)- O-(alkyl), —OH, —NH₂, —CO₂H, —CO₂—(C₂-C₁₂ alkyl), or —C(O)—NH-(alkyl); and
R₅, R₆, R₇, R₈ are each independently —H, halogen, —CN, —CF₃, —OCF₃, -(alkyl), -(alkenyl), -(alkynyl), -(aryl), -(heteroaryl), -(heteroalkyl), -(hydroxyalkyl), —NH₂, —NH-(alkyl), —NH-(alkenyl), —NH-(alkynyl) —NH-(aryl), —NH-(heteroaryl), —OH, —OAc, —CO₂H, —CO₂-(alkyl), —O—C(O) (alkyl), —O-(alkyl), —O-(alkenyl), —O-(alkynyl), —O-(aryl), —O-(heteroaryl), —C(O)—NH₂, —C(O)—NH-(alkyl) or —C(O)—NH-(aryl), or 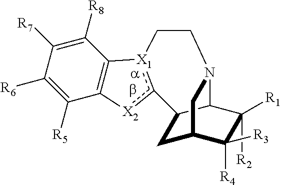
wherein
X₁ is C or N;
X₂ is N, O or S;
α and β are each present or absent and when present each is a bond,
wherein either α or β is present,
when α is present, then X₁ is C and X₂ is S or O, and
when β is present, then X₁ is N and X₂ is N;
R₁, R₂, R₃ and R₄ are each independently H, -(alkyl), -(alkenyl), -(alkynyl), -(aryl), -(heteroaryl), -(heteroalkyl), -(hydroxyalkyl), -(alkyl)-(aryl), -(alkyl)-(heteroaryl), -(alkyl)-OH, -(alkyl)- O-(alkyl), —OH, —NH₂, —CO₂H, —CO₂—(C₂-C₁₂ alkyl) or —C(O)—NH-(alkyl); and
R₅, R₆, R₇, R₈ are each independently —H, halogen, —CN, —CF₃, —OCF₃, -(alkyl), -(alkenyl), -(alkynyl), -(aryl), -(heteroaryl), -(heteroalkyl), -(hydroxyalkyl), —NH₂, —NH-(alkyl), —NH-(alkenyl), —NH-(alkynyl) —NH-(aryl), —NH-(heteroaryl), —OH, —OAc, —CO₂H, —CO₂-(alkyl), —O—C(O) (alkyl), —O-(alkyl), —O-(alkenyl), —O-(alkynyl), —O-(aryl), —O-(heteroaryl), —C(O)—NH₂, —C(O)—NH-(alkyl) or —C(O)—NH-(aryl),
or a pharmaceutically acceptable salt or ester thereof.

22-23. (canceled)

24. The compound of claim 18 having the structure: 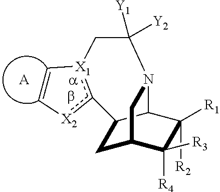
wherein
A is a ring structure, with or without substitution;
X₁ is C or N;
X₂ is N, O or S;
Y₁ is H, -(alkyl), -(alkenyl) or -(alkynyl);
Y₂ is H, -(alkyl), -(alkenyl) or -(alkynyl);
α and β are each present or absent and when present each is a bond,
wherein either α or β is present, and
when α is present, then X₁ is C and X₂ is S or O, or
when β is present, then X₁ is N and X₂ is N; and
R₁, R₂, R₃ and R₄ are each independently H, -(alkyl), -(alkenyl), -(alkynyl), -(aryl), -(heteroaryl), -(heteroalkyl), -(hydroxyalkyl), -(alkyl)-(aryl), -(alkyl)-(heteroaryl), -(alkyl)-OH, -(alkyl)- O-(alkyl), —OH, —NH₂, —CO₂H, —CO₂—(C₂-C₁₂ alkyl) or —C(O)—NH-(alkyl), or 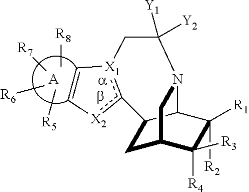
wherein
A is an aryl or heteroaryl;
X₁ is C or N;
X₂ is N, O or S;
Y₁ is H, -(alkyl), -(alkenyl), -(alkynyl), -(cycloalkyl), -(haloalkyl), -(alkyl)-O-(alkyl) or -(alkyl)-(cycloalkyl);
Y₂ is H, -(alkyl), -(alkenyl), -(alkynyl), -(cycloalkyl), -(haloalkyl), -(alkyl)-O-(alkyl) or -(alkyl)-(cycloalkyl);
α and β are each present or absent and when present each is a bond,
wherein either α or β is present,
when α is present, then X₁ is C and X₂ is S or O, and
wherein either α or β is present,
when α is present, then X₁ is C and X₂ is S or O, and
when β is present, then X₁ is N and X₂ is N;
R₁, R₂, R₃ and R₄ are each independently H, -(alkyl), -(alkenyl), -(alkynyl), -(aryl), -(heteroaryl), -(heteroalkyl), -(hydroxyalkyl), -(alkyl)-(aryl), -(alkyl)-(heteroaryl), -(alkyl)-OH, -(alkyl)- O-(alkyl), —OH, —NH₂, —CO₂H, —CO₂—(C₂-C₁₂ alkyl) or —C(O)—NH-(alkyl); and
R₅, R₆, R₇ and R₈ are each independently —H, halogen, —CN, —CF₃, —OCF₃, -(alkyl), -(alkenyl), -(alkynyl), -(aryl), -(heteroaryl), -(heteroalkyl), -(hydroxyalkyl), —NH₂, —NH-(alkyl), —NH-(alkenyl), —NH-(alkynyl) —NH-(aryl), —NH-(heteroaryl), —OH, —OAc, —CO₂H, —CO₂-(alkyl), —O—C(O) (alkyl), —O-(alkyl), —O-(alkenyl), —O-(alkynyl), —O-(aryl), —O-(heteroaryl), —C(O)—NH₂, —C(O)—NH-(alkyl) or C(O)—NH-(aryl), or 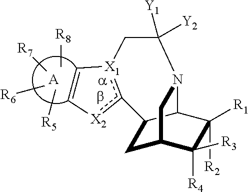
wherein
A is an aryl or heteroaryl;
X₁ is C or N;
X₂ is N, O or S;
Y₁ is H, -(alkyl), -(alkenyl) or -(alkynyl);
Y₂ is H, -(alkyl), -(alkenyl) or -(alkynyl);
α and β are each present or absent and when present each is a bond,
wherein either α or β is present,
when α is present, then X₁ is C and X₂ is S or O, and
when β is present, then X₁ is N and X₂ is N;
R₁, R₂, R₃ and R₄ are each independently H, -(alkyl), -(alkenyl), -(alkynyl), -(aryl), -(heteroaryl), -(heteroalkyl), -(hydroxyalkyl), -(alkyl)-(aryl), -(alkyl)-(heteroaryl), -(alkyl)-OH, -(alkyl)- O-(alkyl), —OH, —NH₂, —CO₂H, —CO₂—(C₂-C₁₂ alkyl) or —C(O)—NH-(alkyl); and
R₅, R₆, R₇ and R₈ are each independently —H, halogen, —CN, —CF₃, —OCF₃, -(alkyl), -(alkenyl), -(alkynyl), -(aryl), -(heteroaryl), -(heteroalkyl), -(hydroxyalkyl), —NH₂, —NH-(alkyl), —NH-(alkenyl), —NH-(alkynyl) —NH-(aryl), —NH-(heteroaryl), —OH, —OAc, —CO₂H, —CO₂-(alkyl), —O—C(O) (alkyl), —O-(alkyl), —O-(alkenyl), —O-(alkynyl), —O-(aryl), —O-(heteroaryl), —C(O)—NH₂, —C(O)—NH-(alkyl) or C(O)—NH-(aryl), or 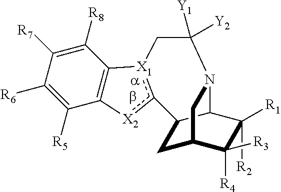
wherein
X₁ is C or N;
X₂ is N, O or S;
Y₁ is H, -(alkyl), -(alkenyl) or -(alkynyl);
Y₂ is H, -(alkyl), -(alkenyl) or -(alkynyl);
α and β are each present or absent and when present each is a bond,
wherein either α or β is present,
when α is present, then X₁ is C and X₂ is S or O, and
when β is present, then X₁ is N and X₂ is N;
R₁, R₂, R₃ and R₄ are each independently H, -(alkyl), -(alkenyl), -(alkynyl), -(aryl), -(heteroaryl), -(heteroalkyl), -(hydroxyalkyl), -(alkyl)-(aryl), -(alkyl)-(heteroaryl), -(alkyl)-OH, -(alkyl)- O-(alkyl), —OH, —NH₂, —CO₂H, —CO₂—(C₂-C₁₂ alkyl) or —C(O)—NH-(alkyl); and
R₅, R₆, R₇, R₈ are each independently —H, halogen, —CN, —CF₃, —OCF₃, -(alkyl), -(alkenyl), -(alkynyl), -(aryl), -(heteroaryl), -(heteroalkyl), -(hydroxyalkyl), —NH₂, —NH-(alkyl), —NH-(alkenyl), —NH-(alkynyl) —NH-(aryl), —NH-(heteroaryl), —OH, —OAc, —CO₂H, —CO₂-(alkyl), —O—C(O) (alkyl), —O-(alkyl), —O-(alkenyl), —O-(alkynyl), —O-(aryl), —O-(heteroaryl), —C(O)—NH₂, —C(O)—NH-(alkyl) or C(O)—NH-(aryl),
or a pharmaceutically acceptable salt or ester thereof.

25-27. (canceled)

28. The compound of claim 24 having the structure: 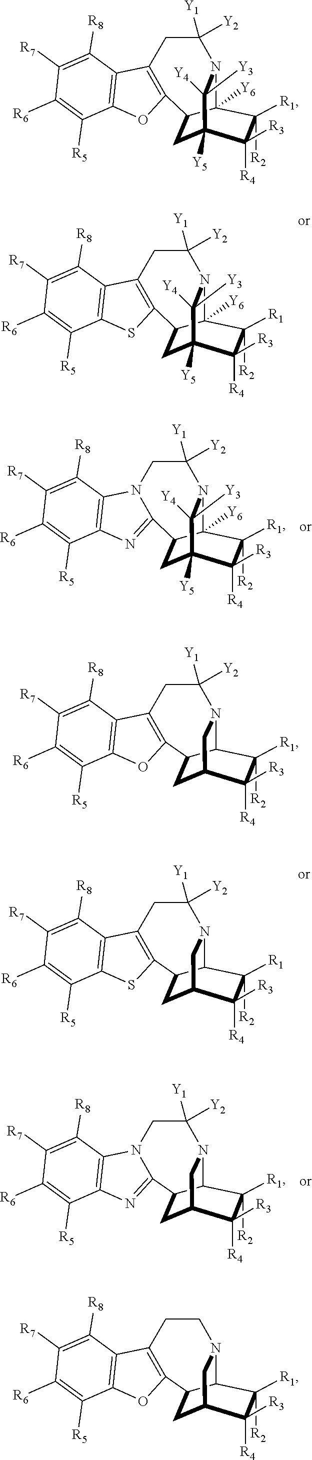 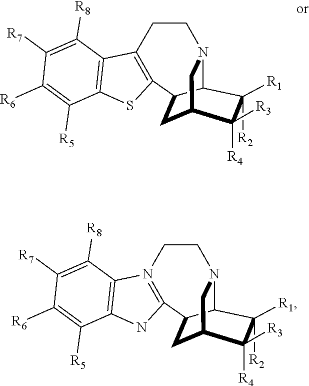
or a pharmaceutically acceptable salt or ester thereof.

29. The compound of claim 28, wherein in the compound R₁ is —H and R₂ is -(alkyl), -(alkenyl), -(alkynyl), -(aryl), -(heteroaryl), -(alkyl)-OH, -(alkyl)-(aryl), -(alkyl)-O-(alkyl), —OH, —NH₂, —CO₂-(alkyl) or —C(O)—NH-(alkyl), or R₂ is —H and R₁ is -(alkyl), -(alkenyl), -(alkynyl), -(aryl), -(heteroaryl), -(alkyl)-OH, -(alkyl)-(aryl), -(alkyl)-O-(alkyl), —OH, —NH₂, —CO₂-(alkyl) or —C(O)—NH-(alkyl), or
wherein R₃ is —H and R₄ is -(alkyl), -(alkenyl), -(alkynyl), -(aryl), -(heteroaryl), -(alkyl)-OH, -(alkyl)-(aryl), -(alkyl)-O-(alkyl), —OH, —NH₂, —CO₂-(alkyl) or —C(O)—NH-(alkyl), or R₄ is —H and R₃ is -(alkyl), -(alkenyl), -(alkynyl), -(aryl), -(heteroaryl), -(alkyl)-OH, -(alkyl)-(aryl), -(alkyl)-O-(alkyl), —OH, —NH₂, —CO₂-(alkyl) or —C(O)—NH-(alkyl), or
wherein R₁ and R₂ are each —H, or R₃ and R₄ are each —H, or R₁, R₂, R₃ and R₄ are each —H, or
wherein in the compound R₅, R₆, R₇ and R₈ are each —H, or
wherein R₅, R₆ and R₇ are each —H and R₈ is halogen, —CN, —CF₃, —OCF₃, -(alkyl), -(alkenyl), -(alkynyl), -(aryl), -(heteroaryl), —NH₂, —NH-(alkyl), —NH-(alkenyl), —NH-(alkynyl) —NH-(aryl), —NH-(heteroaryl), —OH, —OAc, —O—C(O) (alkyl), —O-(alkyl), —O-(alkenyl), —O-(alkynyl), —O-(aryl), —O-(heteroaryl), —C(O)—NH₂, —C(O)—NH-(alkyl) or —C(O)—NH-(aryl), or R₅, R₆ and R₈ are each —H and R₇ is halogen, —CN, —CF₃, —OCF₃, -(alkyl), -(alkenyl), -(alkynyl),-(aryl),-(heteroaryl), —NH₂, —NH-(alkyl), —NH-(alkenyl), —NH-(alkynyl) —NH-(aryl), —NH-(heteroaryl), —OH, —OAc, —O—C(O) (alkyl), —O-(alkyl), —O-(alkenyl), —O-(alkynyl), —O-(aryl), —O-(heteroaryl), —C(O)—NH₂, —C(O)—NH-(alkyl) or —C(O)—NH-(aryl), or
wherein R₅, R₇ and R₈ are each —H and R₆ is halogen, —CN, —CF₃, —OCF₃, -(alkyl), -(alkenyl), -(alkynyl), -(aryl), -(heteroaryl), —NH₂, —NH-(alkyl), —NH-(alkenyl), —NH-(alkynyl) —NH-(aryl), —NH-(heteroaryl), —OH, —OAc, —O—C(O) (alkyl), —O-(alkyl), —O-(alkenyl), —O-(alkynyl), —O-(aryl), —O-(heteroaryl), —C(O)—NH₂, —C(O)—NH-(alkyl) or —C(O)—NH-(aryl), or
R₆, R₇ and R₈ are each —H and R₅ is halogen, —CN, —CF₃, —OCF₃, -(alkyl), -(alkenyl), -(alkynyl), -(aryl), -(heteroaryl), —NH₂, —NH-(alkyl), —NH-(alkenyl), —NH-(alkynyl) —NH-(aryl), —NH-(heteroaryl), —OH, —OAc, —O—C(O) (alkyl), —O-(alkyl), —O-(alkenyl), —O-(alkynyl), —O-(aryl), —O-(heteroaryl), —C(O)—NH₂, —C(O)—NH-(alkyl) or —C(O)—NH-(aryl), or
wherein R₅ and R₈ are each —H and R₆ and R₇ are each independently halogen, —CN, —CF₃, —OCF₃, -(alkyl), -(alkenyl), -(alkynyl), -(aryl),-(heteroaryl), —NH₂, —NH-(alkyl), —NH-(alkenyl), —NH-(alkynyl) —NH-(aryl), —NH-(heteroaryl), —OH, —OAc, —O—C(O) (alkyl), —O-(alkyl), —O-(alkenyl), —O-(alkynyl), —O-(aryl), —O-(heteroaryl), —C(O)—NH₂, —C(O)—NH-(alkyl) or —C(O)—NH-(aryl).

30-35. (canceled)

36. The compound of claim 21 having the structure: 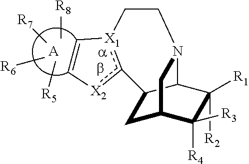
wherein
A is phenyl;
X₁ is C or N;
X₂ is N, O or S;
α and β are each present or absent and when present each is a bond,
wherein either α or β is present,
when α is present, then X₁ is C and X₂ is S or O, and
when β is present, then X₁ is N and X₂ is N;
R₁, R₂, R₃ and R₄ are each independently H, -(alkyl), -(aryl),-(alkyl)-OH, -(alkyl)-(aryl), or -(alkyl)-O-(alkyl), R₅, R₆, R₇ and R₈ are each independently —H, halogen, —OH, —O-(alkyl), —C(O)—NH₂, —C(O)—NH-(alkyl) or —C(O)—NH-(aryl), or 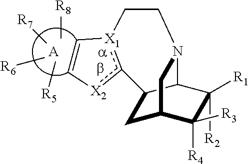
wherein
A is phenyl;
X₁ is C or N;
X₂ is N, O or S;
α and β are each present or absent and when present each is a bond,
wherein either α or β is present,
when α is present, then X₁ is C and X₂ is S or O, and
when β is present, then X₁ is N and X₂ is N;
R₁, R₂, R₃ and R₄ are each independently H, -(alkyl), -(aryl),-(alkyl)-OH, -(alkyl)-(aryl) or -(alkyl)-O-(alkyl), R₅, R₆, R₇ and R₈ are each independently —H, -(alkyl), halogen, —OH, —O-(alkyl), —C(O)—NH₂, —C(O)—NH-(alkyl) or —C(O)—NH-(aryl),
or a pharmaceutically acceptable salt or ester thereof.

37. (canceled)

38. The compound of claim 28, wherein in the compound
Y₁ is H or -(alkyl); and
Y₂ is H or -(alkyl), or Y₁ is H, —CH₃, —CH₂CH₃ or —CH₂CH₂CH₃; and
Y₂ is H, —CH₃, —CH₂CH₃ or —CH₂CH₂CH₃.

39. (canceled)

40. The compound of claim 18 having the structure: 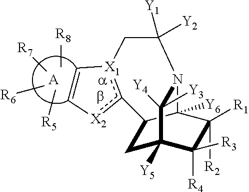
wherein
A is a ring structure, with or without substitution;
X₁ is C or N;
X₂ is N, O or S;
Y₁ is H, -(alkyl), -(alkenyl), -(alkynyl), -(cycloalkyl), -(haloalkyl), -(alkyl)-O-(alkyl) or -(alkyl)-(cycloalkyl);
Y₂ is H, -(alkyl), -(alkenyl), -(alkynyl), -(cycloalkyl), -(haloalkyl), -(alkyl)-O-(alkyl) or -(alkyl)-(cycloalkyl);
Y₃ is H, -(alkyl), -(alkenyl), -(alkynyl), -(cycloalkyl), -(haloalkyl), -(alkyl)-O-(alkyl) or -(alkyl)-(cycloalkyl);
Y₄ is H, -(alkyl), -(alkenyl), -(alkynyl), -(cycloalkyl), -(haloalkyl), -(alkyl)-O-(alkyl) or -(alkyl)-(cycloalkyl);
Y₅ is H, -(alkyl), -(alkenyl), -(alkynyl), -(cycloalkyl), -(haloalkyl), -(alkyl)-O-(alkyl) or -(alkyl)-(cycloalkyl);
Y₆ is H, -(alkyl), -(alkenyl), -(alkynyl), -(cycloalkyl), -(haloalkyl), -(alkyl)-O-(alkyl) or -(alkyl)-(cycloalkyl);
α and β are each present or absent and when present each is a bond,
wherein either α or β is present, and
when α is present, then X₁ is C and X₂ is S or O, or
when β is present, then X₁ is N and X₂ is N; and
R₁, R₂, R₃ and R₄ are each independently H, -(alkyl), -(alkenyl),-(alkynyl), -(aryl), -(heteroaryl), -(heteroalkyl), -(hydroxyalkyl), -(alkyl)-(aryl), -(alkyl)-(heteroaryl), -(alkyl)-OH, -(alkyl)- O-(alkyl), —OH, —NH₂, —CO₂H, —CO₂—(C₂-C₁₂ alkyl) or —C(O)—NH-(alkyl), or 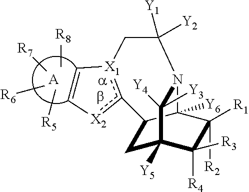
wherein
A is a ring structure, with or without substitution;
X₁ is C or N;
X₂ is N, O or S;
Y₁ is H, -(alkyl), -(alkenyl), -(alkynyl), -(cycloalkyl) or -(alkyl)-(cycloalkyl);
Y₂ is H, -(alkyl), -(alkenyl), -(alkynyl), -(cycloalkyl) or -(alkyl)-(cycloalkyl);
Y₃ is H, -(alkyl), -(alkenyl), -(alkynyl), -(cycloalkyl) or -(alkyl)-(cycloalkyl);
Y₄ is H, -(alkyl), -(alkenyl), -(alkynyl), -(cycloalkyl) or -(alkyl)-(cycloalkyl);
Y₅ is H, -(alkyl), -(alkenyl), -(alkynyl), -(cycloalkyl) or -(alkyl)-(cycloalkyl);
Y₆ is H, -(alkyl), -(alkenyl), -(alkynyl), -(cycloalkyl) or -(alkyl)-(cycloalkyl);
α and β are each present or absent and when present each is a bond,
wherein either α or β is present, and
when α is present, then X₁ is C and X₂ is S or O, or
when β is present, then X₁ is N and X₂ is N; and
R₁, R₂, R₃ and R₄ are each independently H, -(alkyl), -(alkenyl), -(alkynyl), -(aryl), -(heteroaryl), -(heteroalkyl), -(hydroxyalkyl), -(alkyl)-(aryl), -(alkyl)-(heteroaryl), -(alkyl)-OH, -(alkyl)- O-(alkyl), —OH, —NH₂, —CO₂H, —CO₂—(C₂-C₁₂ alkyl) or —C(O)—NH-(alkyl), or 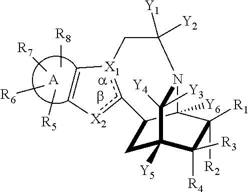
wherein
A is an aryl or heteroaryl;
X₁ is C or N;
X₂ is N, O or S;
Y₁ is H, -(alkyl), -(alkenyl), -(alkynyl), -(cycloalkyl) or -(alkyl)-(cycloalkyl);
Y₂ is H, -(alkyl), -(alkenyl), -(alkynyl), -(cycloalkyl) or -(alkyl)-(cycloalkyl);
Y₃ is H, -(alkyl), -(alkenyl), -(alkynyl), -(cycloalkyl) or -(alkyl)-(cycloalkyl);
Y₄ is H, -(alkyl), -(alkenyl), -(alkynyl), -(cycloalkyl) or -(alkyl)-(cycloalkyl);
Y₅ is H, -(alkyl), -(alkenyl), -(alkynyl), -(cycloalkyl) or -(alkyl)-(cycloalkyl);
Y₆ is H, -(alkyl), -(alkenyl), -(alkynyl), -(cycloalkyl) or -(alkyl)-(cycloalkyl);
α and β are each present or absent and when present each is a bond,
wherein either α or β is present,
when α is present, then X₁ is C and X₂ is S or O, and
when β is present, then X₁ is N and X₂ is N;
R₁, R₂, R₃ and R₄ are each independently H, -(alkyl), -(alkenyl), -(alkynyl), -(aryl), -(heteroaryl), -(heteroalkyl), -(hydroxyalkyl), -(alkyl)-(aryl), -(alkyl)-(heteroaryl), -(alkyl)-OH, -(alkyl)- O-(alkyl), —OH, —NH₂, —CO₂H, —CO₂—(C₂-C₁₂ alkyl) or —C(O)—NH-(alkyl); and
R₅, R₆, R₇, R₈ are each independently —H, halogen, —CN, —CF₃, —OCF₃, -(alkyl), -(alkenyl), -(alkynyl), -(aryl), -(heteroaryl), -(heteroalkyl), -(hydroxyalkyl), —NH₂, —NH-(alkyl), —NH-(alkenyl), —NH-(alkynyl) —NH-(aryl), —NH-(heteroaryl), —OH, —OAc, —CO₂H, —CO₂-(alkyl), —O—C(O) (alkyl), —O-(alkyl), —O-(alkenyl), —O-(alkynyl), —O-(aryl), —O-(heteroaryl), —C(O)—NH₂, —C(O)—NH-(alkyl) or —C(O)—NH-(aryl),
or a pharmaceutically acceptable salt or ester thereof.

41-43. (canceled)

44. The compound of claim 18 having the structure: 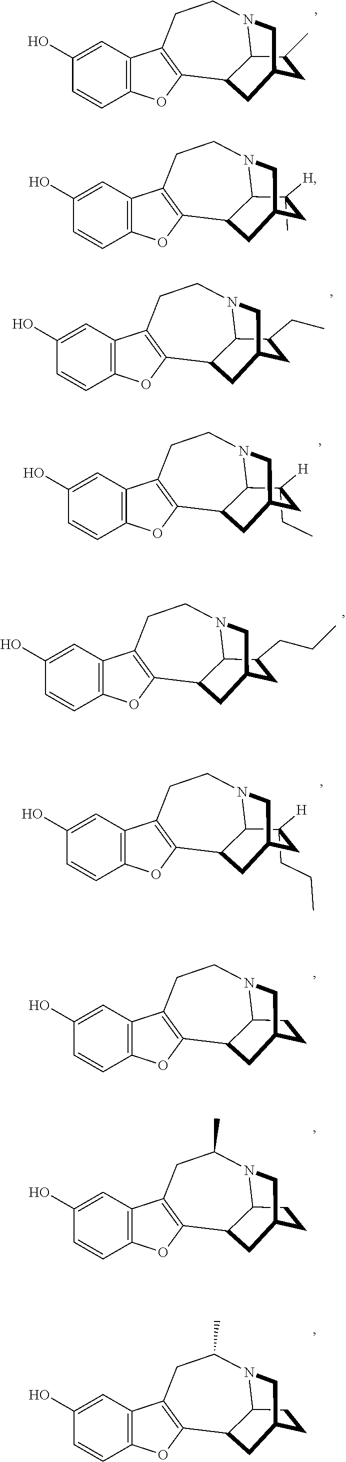 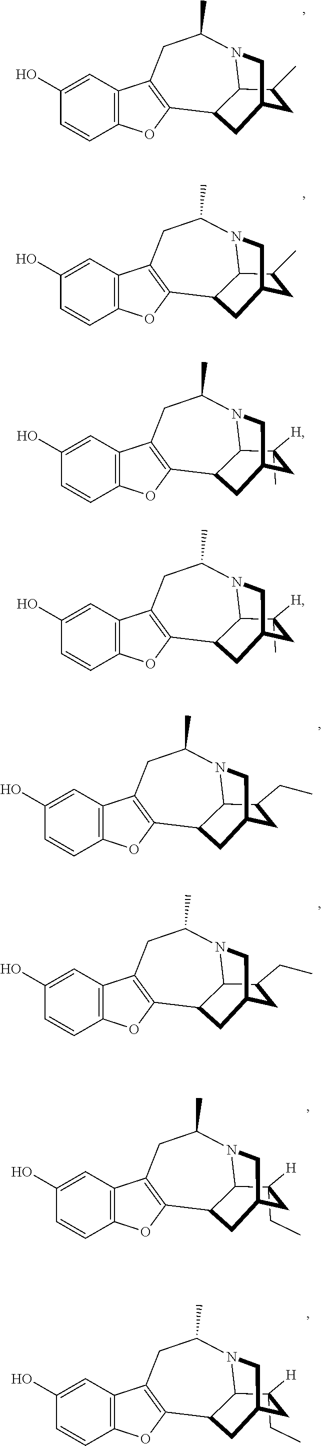 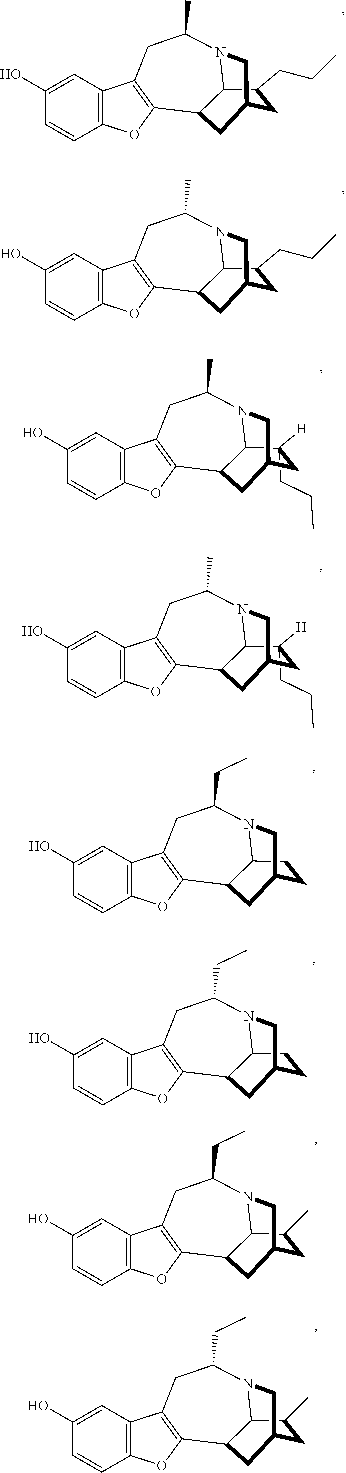 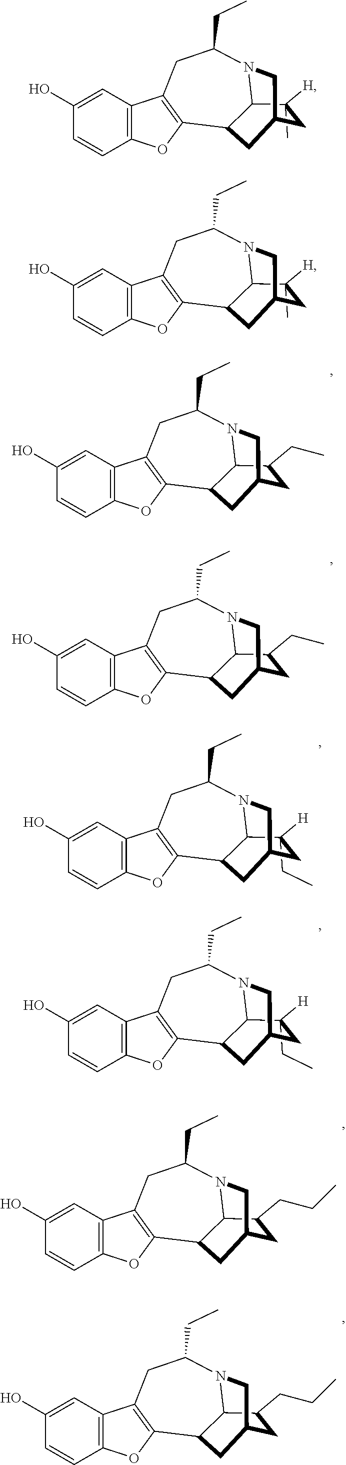 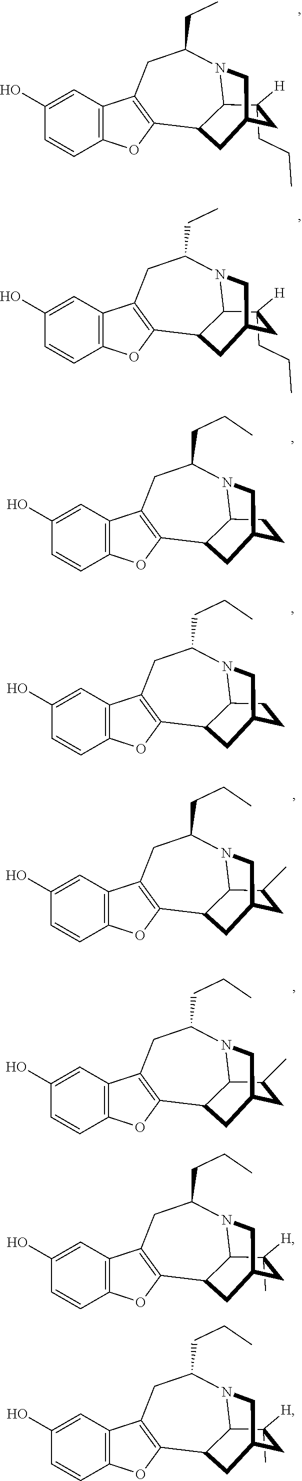 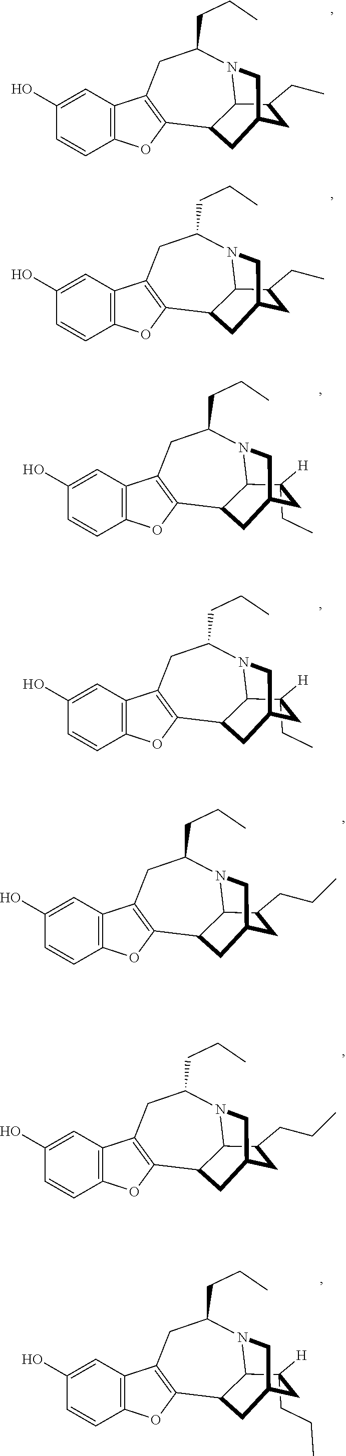 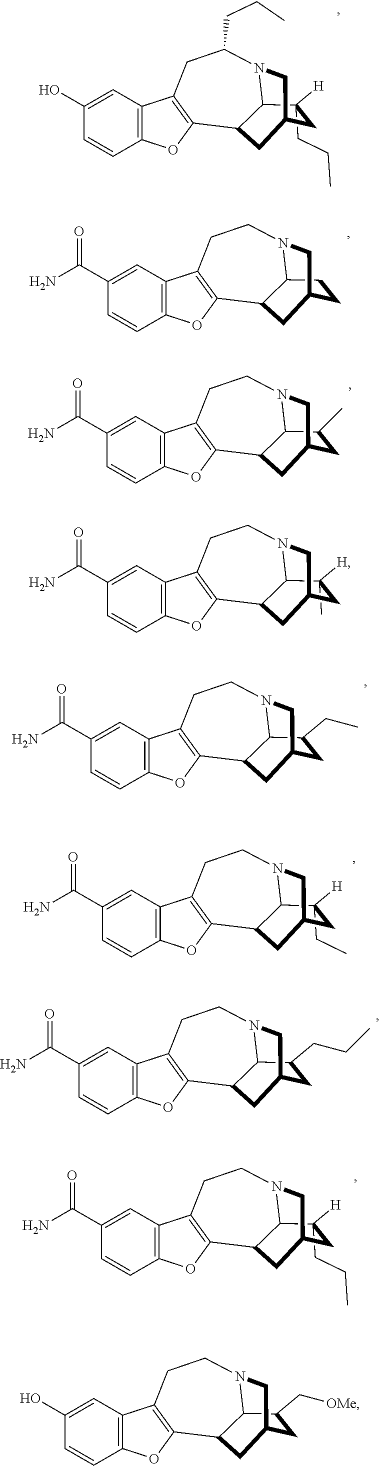 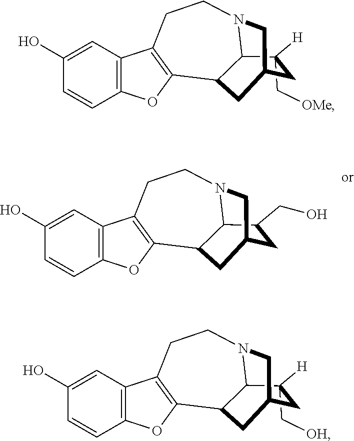
or a pharmaceutically acceptable salt or ester thereof.

45. A pharmaceutical composition comprising the compound of claim 44 and a pharmaceutically acceptable carrier.

46. A method of activating mu-opioid receptor, delta-opioid receptor and/or kappa-opioid receptor comprising contacting the mu-opioid receptor, delta-opioid receptor and/or kappa-opioid receptor with the composition of claim 45; or
a method of inhibiting mu-opioid receptor, delta-opioid receptor and/or kappa-opioid receptor comprising contacting the mu-opioid receptor, delta-opioid receptor and/or kappa-opioid receptor with the composition of claim 45; or
a method of inhibiting serotonin transporter (SERT) comprising contacting the serotonin transporter (SERT) with the composition of claim 45, or
a method of treating a subject afflicted with depression, major depression, pain, a mood disorder, anxiety disorder, obsessive-compulsive disorder (OCD) or stress disorder comprising administering to the subject the composition of claim 45 comprising an effective amount of the compound, so as to thereby treat the subject afflicted with depression, major depression, pain, anxiety disorder, obsessive-compulsive disorder (OCD) or stress disorder, or
a method of altering the psychological state of a subject comprising administering to the composition of claim 45 comprising an effective amount of the compound, so as to thereby alter the psychological state of the subject, or
a method of enhancing the effect of psychotherapy in a subject comprising administering to the subject the composition of claim 45 comprising an effective amount of the compound, so as to thereby enhance the effect of the psychotherapy in the subject, or
a method of treating a subject afflicted with Parkinson's disease, or traumatic brain injury comprising administering to the subject the composition of claim 45 comprising an effective amount of the compound, so as to thereby treat the subject afflicted with Parkinson's disease or traumatic brain injury, or
a method of treating a subject afflicted with a headache or a migraine comprising administering to the subject the composition of claim 45 comprising an effective amount of the compound, so as to thereby treat the subject afflicted with the headache or the migraine, or
a method of treating a subject afflicted with a substance use disorder comprising administering to the subject the composition of claim 45 comprising an effective amount of the compound, so as to thereby treat the subject afflicted with the substance use disorder, or
a method of treating a subject afflicted with opioid withdrawal symptoms comprising administering to the subject the composition of claim 45 comprising an effective amount of the compound, so as to thereby treat the subject afflicted with the opioid withdrawal symptoms, or
a method of treating a subject afflicted with a symptom of substance use disorder comprising administering to the subject the composition of claim 45 comprising an effective amount of the compound, so as to thereby treat the subject afflicted with the symptom of substance use disorder,
wherein a symptom of substance use disorder is opioid withdrawal, mitigation of relapse to opioid use or SUD, hyperalgesia or allodynia.

47-52. (canceled)

53. The method of claim 46, wherein the substance use disorder is opioid use disorder, alcohol use disorder or stimulant use disorder including nicotine use disorder,
wherein the substance is an opioid, and
wherein the opioid is morphine, hydromorphone, oxymorphone, codeine, dihydrocodeine, hydrocodone, oxycodone, nalbuphine, butorphanol, etorphine, dihydroetorphine, levorphanol, metazocine, pentazocine, meptazinol, meperidine (pethidine), buprenorphine, methadone, tramadol, tapentadol, mitragynine, 3-deutero-mitragynine, 7-hydroxymitragynine, 3-deutero-7-hydroxymitragynine, mitragynine pseudoindoxyl or tianeptine; or
wherein the opioid is fentanyl, sufentanil, alfentanil, furanylfentanyl, 3-methylfentanyl, valerylfentanyl, butyrylfentanyl, R-Hydroxythiofentanyl, acrylfentanyl or carfentanil, or
wherein the stimulant is cocaine, amphetamine, methamphetamine, cathinone and its derivatives, or nicotine.

54-59. (canceled)

60. The method of claim 53, wherein the risk of relapse to the use of opioids, alcohol or stimulants is reduced; or
wherein self-administration of an opioid, alcohol or stimulant is reduced,
wherein the treating is effective for an extended time,
wherein the time is 1-5 days or 1-5 weeks,
wherein the effective amount of the compound administered to the subject without inducing cardiotoxicity, without inducing QT interval prolongation or without inducing cardiac arrhythmia.

61-63. (canceled)

64. The method claim 60, wherein the subject is a mammal,
wherein the mammal is a human,
wherein the effective amount of 10-500 mg of the compound is administered to the subject.

65-66. (canceled)
